SEQUENCE LISTING (1 sequence)

SEQ ID NO: 1 - protein, 595 aa, Homo sapiens [sequence omitted]

(19) United States
(12) Patent Application Publication
(10) Pub. No.: US 2025/0381179 A1
(43) Pub. Date: Dec. 18, 2025

(54) CHROMAN-BASED COMPOUNDS FOR TREATING CANCER

(71) Applicant: Accutar Biotechnology Inc., Cranbury, NJ (US)

(72) Inventors: Jie Fan, New York, NY (US); Wei He, Zionsville, IN (US); Ke Liu, Bellevue, WA (US); Hui Zhang, Cambridge, MA (US); Su Kim, Chicago, IL (US)

(21) Appl. No.: 19/216,567

(22) Filed: May 22, 2025

Related U.S. Application Data
(60) Provisional application No. 63/650,793, filed on May 22, 2024.
(60) Provisional application No. 63/691,567, filed on Sep. 6, 2024.

(51) Int. Cl. A61K 31/496; A61P 35/00; C07D 405/14

(52) U.S. Cl. CPC A61K 31/496; A61P 35/00; C07D 405/14

(57) ABSTRACT

The present disclosure relates to compounds and their uses for treating cancers, including breast cancer, in a subject in need of treatment, comprising administering at least one compound disclosed herein.

### SEQUENCE LISTING

The instant application contains a Sequence Listing which has been submitted electronically in XML format and is hereby incorporated by reference in its entirety. Said XML copy, created on Jul. 3, 2025, is named ACT-036US_SL.xml and is 2,453 bytes in size.

### BACKGROUND

Estrogen receptor signaling is a critical regulator of cell proliferation, differentiation, and survival in hormone-sensitive cancers, such as breast cancer, endometrial cancer, and ovarian cancer.

Breast cancer is the most frequent cancer diagnosed in women worldwide. According to Cancer Statistics 2020 (see Siegel et el., CA: A Cancer Journal for Clinicians, 2020, 70 (1), 7-30), breast cancer represents 30% of female cancers with 276,480 estimated new cases and more than 42,000 estimated deaths in 2020. The disease can be classified into four molecular subtypes—luminal A, luminal B, human epidermal growth factor receptor 2 (HER2), and triple-negative breast cancer (TNBC)—according to the expression of the estrogen receptor (ER) and the progesterone receptor (PR), and the overexpression of the HER2. For metastatic breast cancer, the 5-year relative cancer-specific survival rate is low: 29% regardless of subtype and can drop to 12% for metastatic TNBC, (i.e., ER-negative, PR-negative, and HER2-negative). This shows that strategies of treatment for metastatic breast cancer patients are not effective enough to ensure a good survival rate (see Burguin et al., Journal of Personalized Medicine, 2021, 11, 808). ER-positive/HER2-negative breast cancer is defined by the presence of ER and/or PR gene amplification and the absence of HER2 gene amplification. In particular, hormone receptor (HR)-positive/HER2-negative breast cancer accounts for 65%-70% of all breast cancers, and the incidence increases with increasing age (see Huppert et al., A Cancer Journal for Clinicians, 2023, 480-515).

Because ER plays a critical role in breast cancer initiation and proliferation, modulation of estrogen and estrogen activity is the standard of care (SOC) therapy for patients with ER-positive breast cancer. Endocrine therapy targets the ER directly, and common types of endocrine therapy include selective estrogen receptor modulators (SERMs), selective estrogen receptor degraders (SERDs), and aromatase inhibitors (see Howlader et al. Journal of the National Cancer Institute, 2014, 106; El Sayed et al. Frontiers in Oncology, 2019, 9, 510). SERMs include tamoxifen, toremifene, bazedoxifene, and raloxifene, which are antiestrogens that compete with estrogen by binding to the ER.

In contrast to SERMs, SERDs (such as fulvestrant) completely block the ER signaling pathway. Fulvestrant blocks ER dimerization and DNA binding, increases ER turnover, and inhibits nuclear uptake of the receptor. Fulvestrant also binds to ER with a higher affinity than tamoxifen. Fulvestrant monthly intramuscular injection was approved in 2002 by the FDA for the treatment of HR-positive metastatic breast cancer in postmenopausal women with disease progression following antiestrogen therapy (see Bross et al., Oncologist, 2002, 477-80). However, fulvestrant's route of administration contributes to poor drug exposure limiting efficacy. It has also been reported that up to 50% of ER baseline levels remained after 6 months of fulvestrant treatment (see He at el., Cancer Research, 2021, 81, PS18-09). In 2017, fulvestrant was approved as first-line monotherapy for advanced ER-positive breast cancer. But the combination of fulvestrant with other endocrine therapies has not shown any advantages over fulvestrant used in monotherapy (see Bergh et al., Journal of Clinical Oncology, 30, 2012). FDA approved in 2023 an oral SERD, clacestrant, for the treatment of ER-positive, HER2 negative, ESR 1 mutated advanced or metastatic breast cancer. Additional SERDs in clinical development include GDC9545, AZD9833, SAR439859, palazestrant, rintodestrant, and ZN-C5.

ER therapies are generally limited by adverse effects and development of resistance. For example, the most frequent adverse events of SERMs are hot flushes, nausea, vomiting, vaginal bleeding/discharges, and increased risk of thromboembolic events. In particular, about 40% of HR-positive breast cancer patients will develop resistance to SERMs. SERMs resistance can occur by the loss of ER expression or functions. A potential mechanism for ER expression loss is the overpopulation of ER-negative cells in heterogenous ER-positive tumors. Mutations in the ligand-binding domain of ER gene (ESR1) inhibit the binding of estrogen to the ER, leading to the abolition of downstream signaling. Moreover, abnormal splicing can lead to truncated, nonfunctional ER protein. Another explanation for SERMs resistance is the abnormal expression of ER coregulators (see Burguin et al., Journal of Personalized Medicine, 2021, 11, 808). While SERDs have improved progression-free survival in this patient population, as monotherapy they have shown very limited overall response rates (ORR).

Accordingly, there remains a need for effective and safe therapeutic agents. In particular, there is a need for effective compounds and methods for treating or preventing cancers, such as ER-mediated cancers (e.g., breast cancer), with compounds that have, for example, improved bioavailability (e.g., orally bioavailable compounds), safety, efficacy, while at the same time reducing the adverse events and risks to patients.

### SUMMARY

The present disclosure is directed, in some embodiments, to methods of treating ER-mediated cancers, such as breast cancer, with the compounds disclosed herein. In some embodiments, the disclosure is directed to a method of treating an ER-mediated cancer, such as breast cancer, in a patient in need thereof, comprising administering to the patient compounds disclosed herein, such as Protein-Protein Interaction Targeted Chimeras (PPI-TACs). PPI-TACs possess many advantages over conventional inhibitors and/or degraders (e.g., Proteolysis-targeting chimeras), such as an enhanced degradation potency and/or selectivity for their targets. For example, PPI-TACs rely only on proximity by projecting one small molecule simultaneously to a targeted protein and E3 ligase and are also believed to direct protein-protein interactions between the targeted protein and E3 ligase, thus leading to enhanced degradation potency and selectivity. In some embodiments, the PPI-TACs work sub-stoichiometrically by inducing multiple rounds of degradation of target proteins. This is attributed to the PPI-TAC molecule being released from the proteosome-degraded protein to bind another target protein and E3 ubiquitin ligase, which in turn results in a greater potency compared to each isolated moiety binding to its respective target.

In some embodiments, PPI-TACs disclosed herein (e.g., compounds of Formula 1) can deplete target proteins that are not responsive to biochemical inhibition by binding accessible pockets that do not affect the biochemical activity of the target but still permit their degradation. It is believed that the PPI-TACs disclosed herein may achieve improved degradation selectivity and degradation potency due to the induced protein-protein interactions. In some embodiments, the chimeric degraders (e.g., compounds of Formula 1) bind, ubiquitinate and degrade ERα by bringing ERα into close proximity with an E3 ligase. This mechanism of action is believed to result in greater specificity and more complete target blockade compared to, for example, SERDs.

In some embodiments, the cancer treated by the present methods is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer. In some embodiments, the cancer is breast cancer, lung cancer, prostate cancer, pancreatic cancer, or ovarian cancer. In some embodiments, the cancer is breast cancer, lung cancer, or prostate cancer.

In some embodiments, the cancer treated by the present methods is breast cancer. In embodiments, the breast cancer is metastatic or locally advanced. In some embodiments, the breast cancer is estrogen receptor positive (ER+) breast cancer (e.g., human epidermal growth factor receptor 2 negative (HER2−)).

In some embodiments, the subject treated by the present methods is a human (e.g., a patient).

In some embodiments, the disclosure is directed to a method of treating cancer, such as breast cancer, in a subject (e.g., patient) in need thereof, comprising administering to the patient a therapeutically effective amount of a compound or a mixture of compounds, wherein the compound is represented by Formula 1 or is a pharmaceutically acceptable salt thereof:

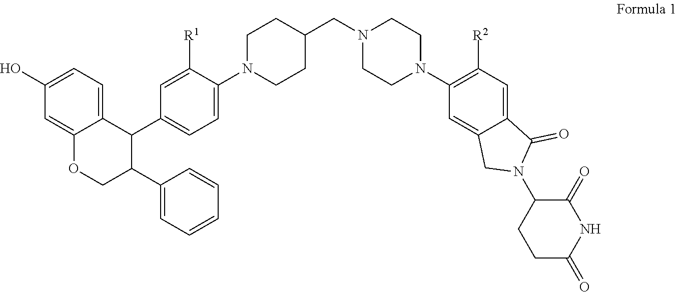

wherein:
R¹ is selected from H, Cl, Br, and F, and
R² is H or F, and
wherein the therapeutically effective amount of the compound of Formula 1 is 20 mg to 1200 mg. In some embodiments, the therapeutically effective amount of the compound of Formula 1 is 50 to 150 mg, such as 100 mg. In some embodiments, the therapeutically effective amount of the compound of Formula 1 is 150 to 250 mg, such as 200 mg. In some embodiments, the therapeutically effective amount of the compound of Formula 1 is 250 to 350 mg, such as 300 mg. In some embodiments, the therapeutically effective amount of the compound of Formula 1 is 350 to 450 mg, such as 400 mg. In some embodiments, the therapeutically effective amount of the compound of Formula 1 is 450 to 550 mg, such as 500 mg. In some embodiments, the therapeutically effective amount of the compound of Formula 1 is 550 to 650 mg, such as 600 mg.

In some embodiments, R¹ and R² are both H. In some embodiments, R¹ and R² are both F. In some embodiments, R¹ is H and R² is F. In some embodiments, R¹ is F and R² is H.

In some embodiments, the compound of Formula 1 is selected from

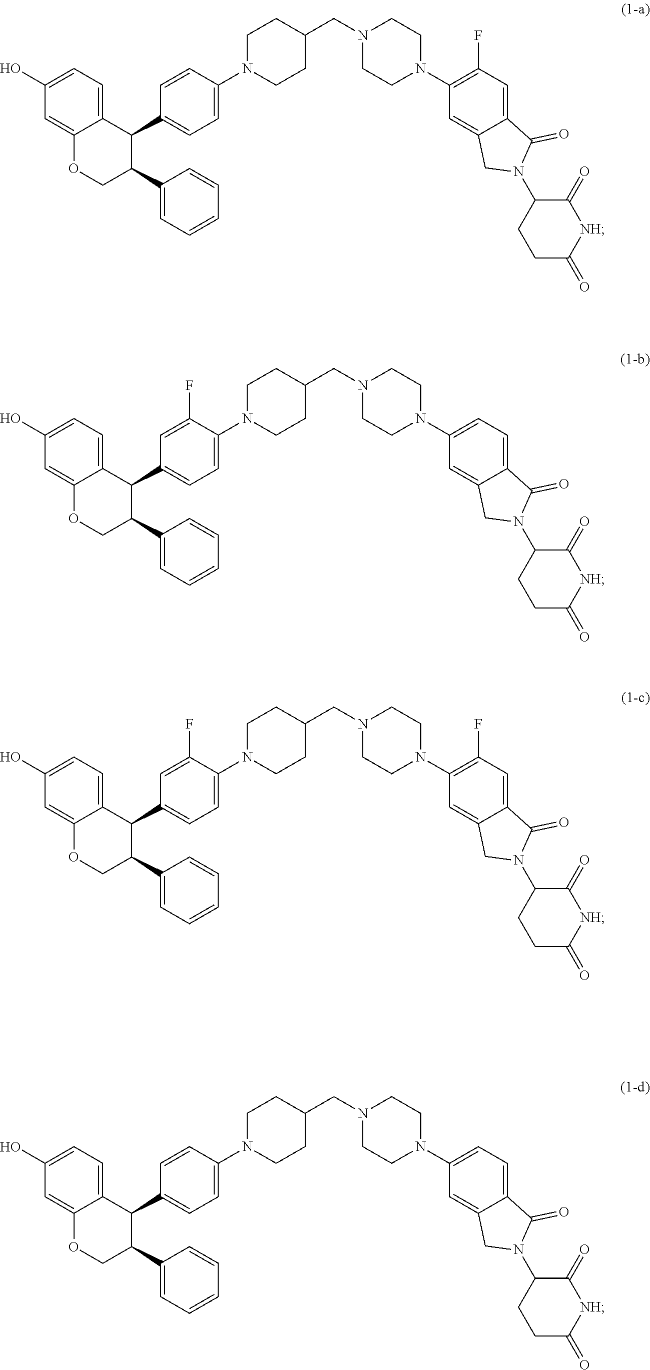 [SMILES: O=C1CCC(N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

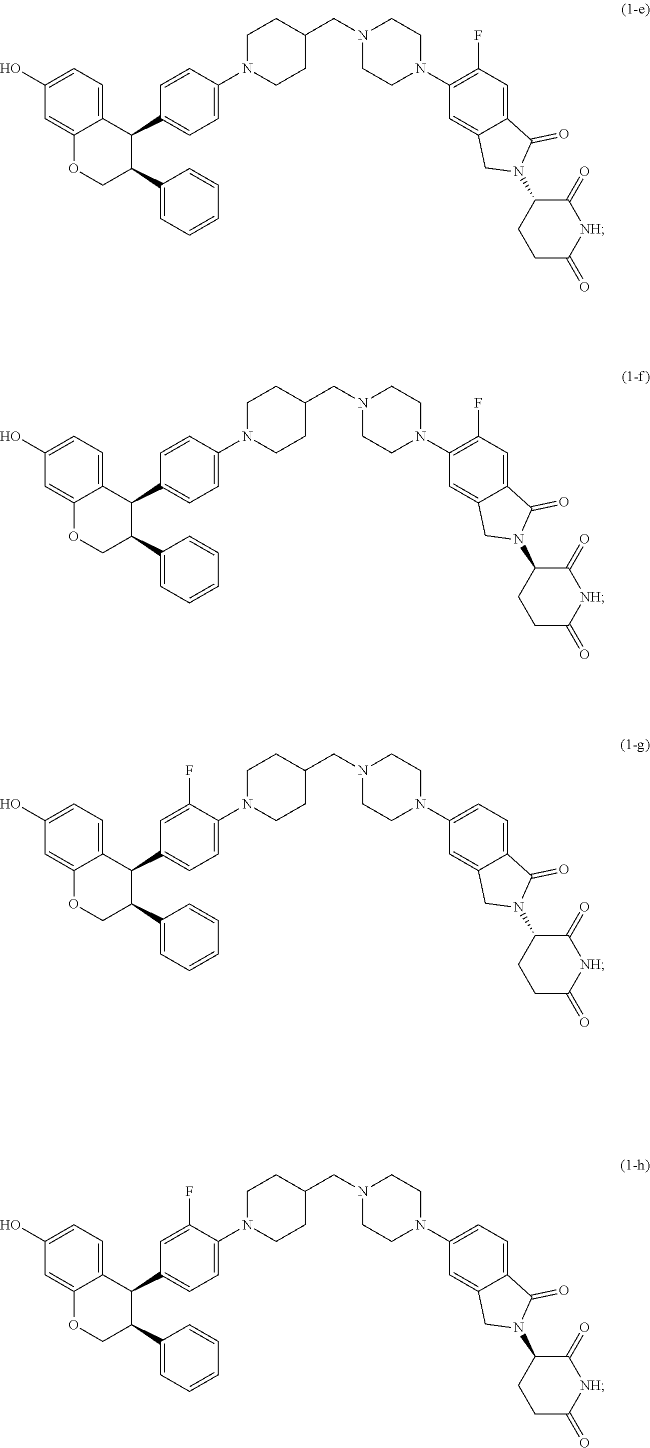 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

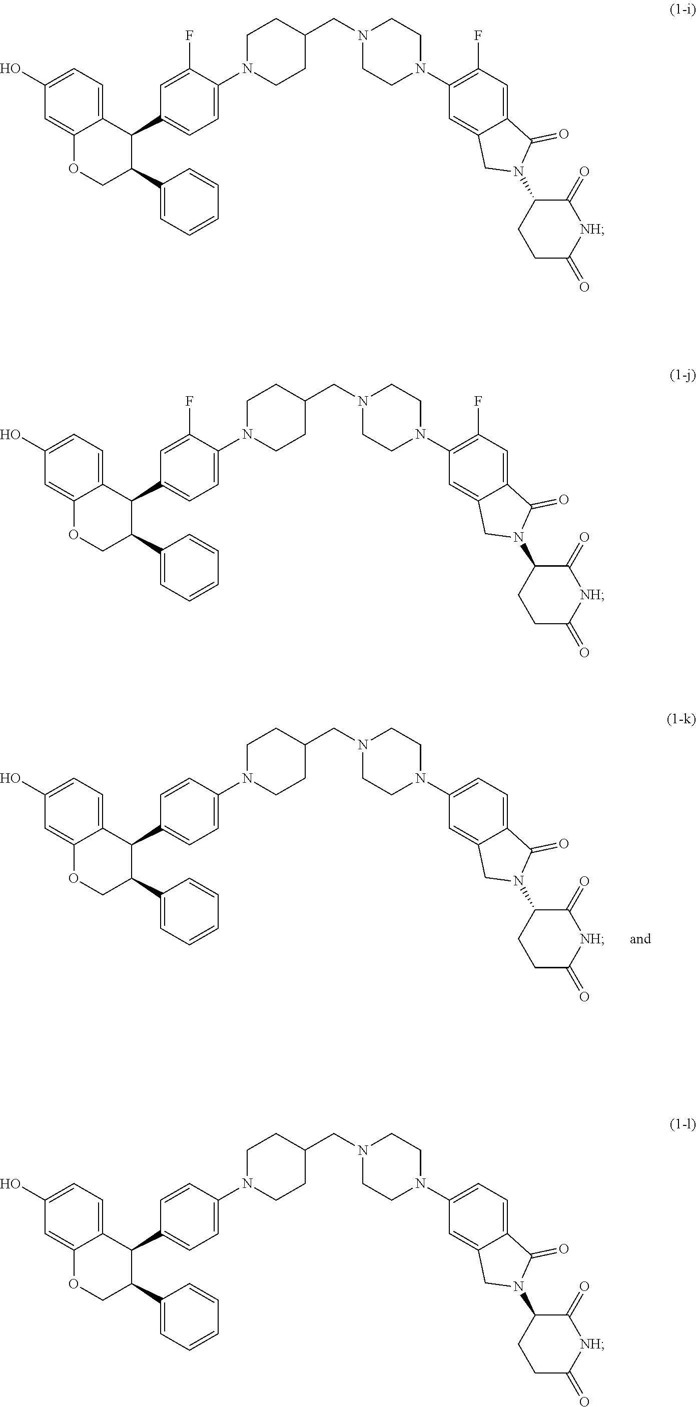 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

or is a combination thereof.

In some embodiments, the present disclosure provides a compound, wherein the compound is represented by Formula 1 or is a pharmaceutically acceptable salt thereof, and wherein the compound is selected from

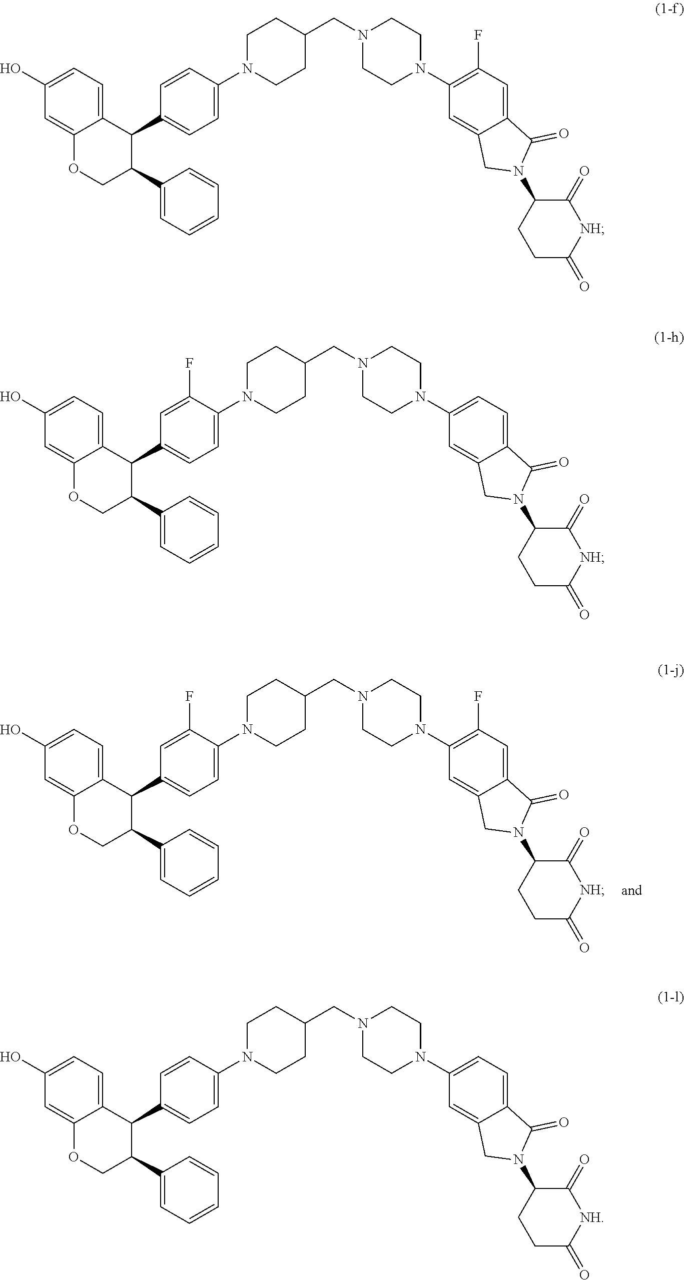 [SMILES: O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

In some embodiments, the present disclosure provides a mixture of the following compounds

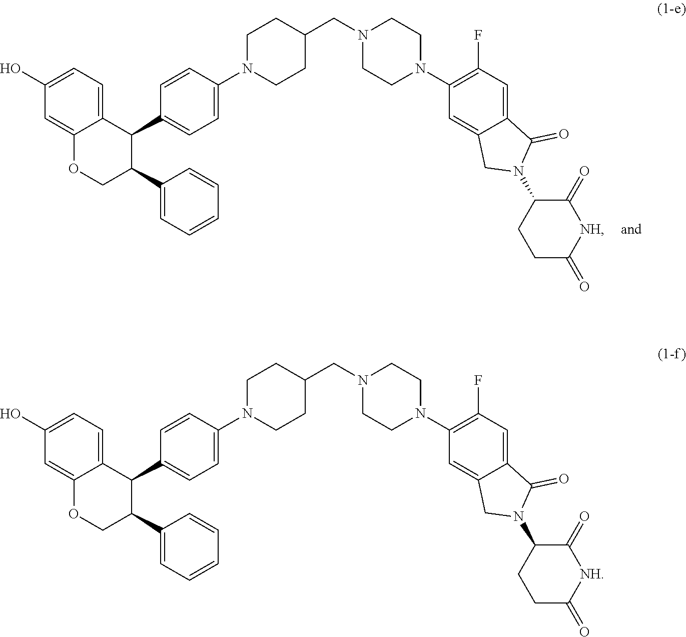 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1]

In some embodiments, the present disclosure provides a mixture of the following compounds

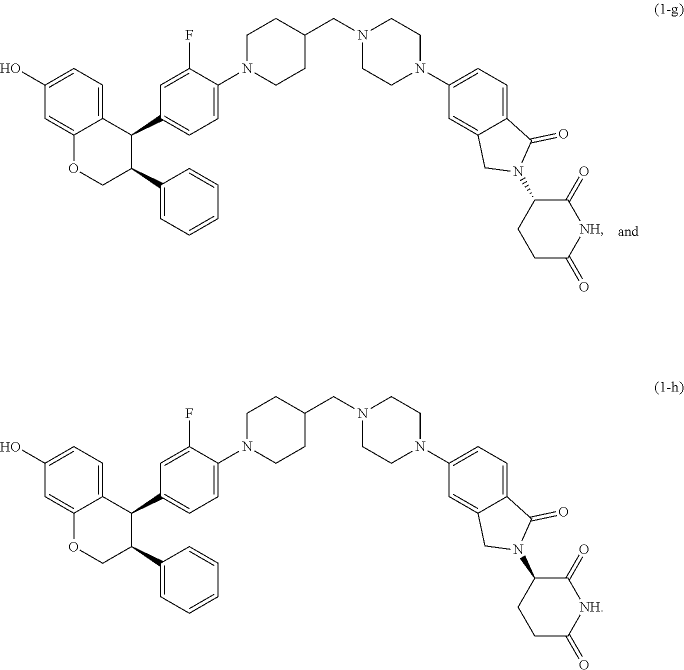 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

In some embodiments, the present disclosure provides a mixture of the following compounds

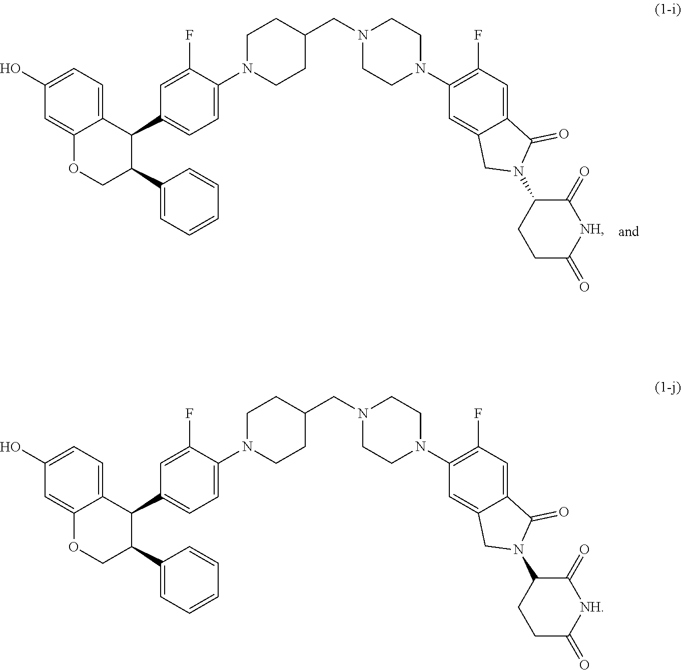

In some embodiments, the present disclosure provides a mixture of the following compounds

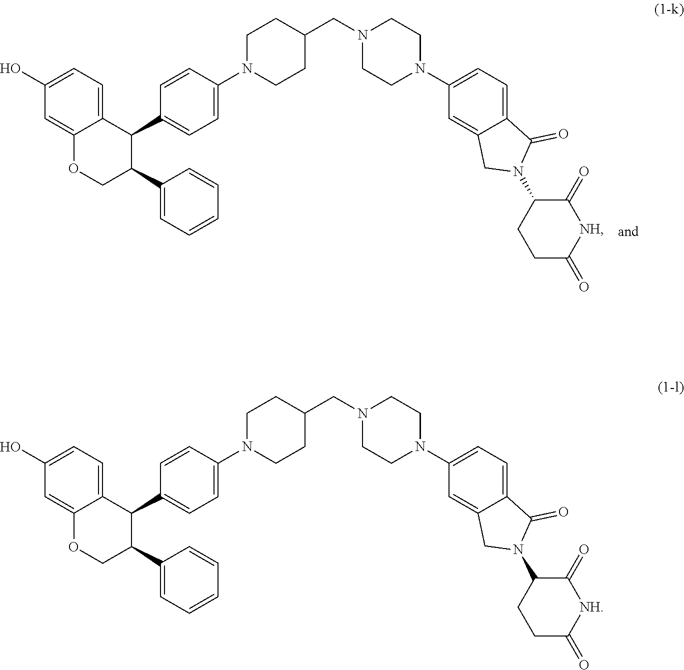

In some embodiments, the mixture comprises two compounds disclosed herein. In some embodiments, the mixture comprises varying amounts (e.g., relative amounts of compound (1-k) and (1-1)) for the two compounds present in the mixture. In some embodiments, the relative amounts of compound (1-e) and (1-f) is greater than 95:5, such as greater than 98:2 or 99:1. In some embodiments, the relative amounts of compound (1-e) and (1-f) is greater than 90:10. In some embodiments, the relative amounts of compound (1-g) and (1-h) is greater than 95:5, such as greater than 98:2 or 99:1. In some embodiments, the relative amounts of compound (1-g) and (1-h) is greater than 90:10. In some embodiments, the relative amounts of compound (1-i) and (1-j) is greater than 95:5, such as greater than 98:2 or 99:1. In some embodiments, at the relative amounts of compound (1-i) and (1-j) is greater than 90:10. In some embodiments, the relative amounts of compound (1-k) and (1-1) is greater than 95:5, such as greater than 98:2 or 99:1. In some embodiments, the relative amounts of compound (1-k) and (1-1) is greater than 90:10.

In some embodiments, the disclosure is directed to a method of treating breast cancer in a patient in need thereof, wherein the breast cancer comprises an Estrogen Receptor 1 (ESR1) tumor mutation or ESR1-wild-type, the method comprising:
orally administering to the patient once daily a therapeutically effective amount of a compound, wherein the compound is selected from the following:

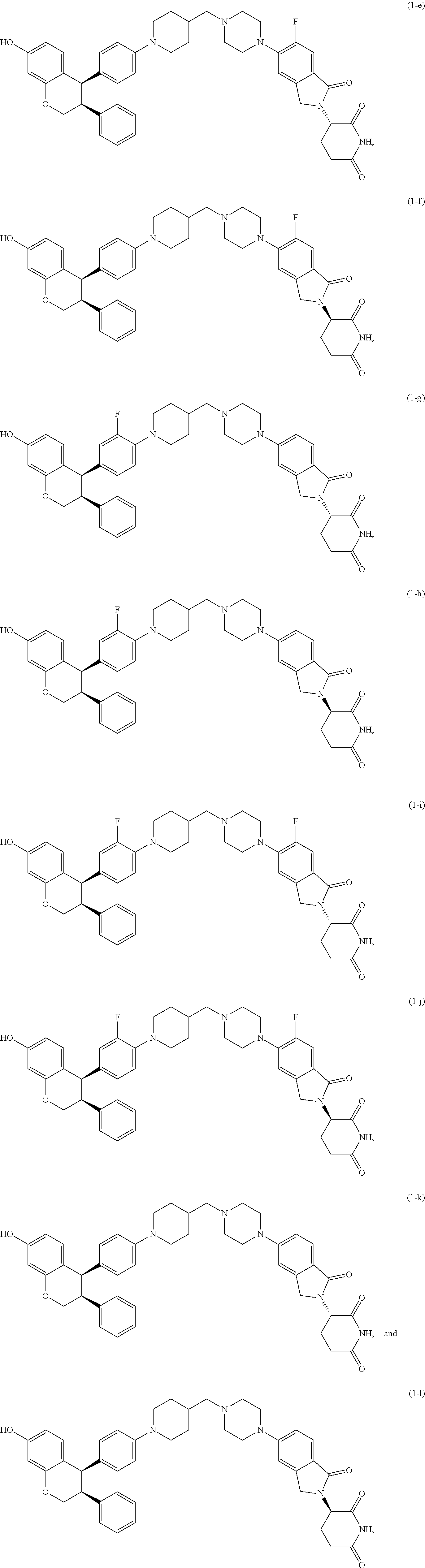

or is a pharmaceutically acceptable salt thereof.

In some embodiments, the compounds disclosed herein inhibit and/or degrade wild-type and mutant ER activity.

In some embodiments, the compound is (1-e). In some embodiments, the compound is (1-f). In some embodiments, the compound is (1-g). In some embodiments, the compound is (1-h). In some embodiments, the compound is (1-i). In some embodiments, the compound is (1-j). In some embodiments, the compound is (1-k). In some embodiments, the compound is (1-1).

In some embodiments, the therapeutically effective amount of the compound is administered to the subject once a day or twice a day. In some embodiments, the therapeutically effective amount of the compound is administered to the subject all at once or is administered in two, three, or four unit doses. In some embodiments, the therapeutically effective amount of the compound is 50 mg to 1000 mg. In some embodiments, the therapeutically effective amount of the compound is 50 mg to 1000 mg. In some embodiments, the therapeutically effective amount of the compound is 100 mg to 600 mg. In some embodiments, the therapeutically effective amount of the compound is 100 mg. In some embodiments, the therapeutically effective amount of the compound is 200 mg. In some embodiments, the therapeutically effective amount of the compound is 250 mg. In some embodiments, the therapeutically effective amount of the compound is 300 mg. In some embodiments, the therapeutically effective amount of the compound is 350 mg. In some embodiments, the therapeutically effective amount of the compound is 400 mg. In some embodiments, the therapeutically effective amount of the compound is 450 mg. In some embodiments, the therapeutically effective amount of the compound is 500 mg. In some embodiments, the therapeutically effective amount of the compound is 550 mg. In some embodiments, the therapeutically effective amount of the compound is 600 mg. In some embodiments, the therapeutically effective amount of the compound is 650 mg. In some embodiments, the therapeutically effective amount of the compound is 700 mg. In some embodiments, the therapeutically effective amount of the compound is 750 mg. In some embodiments, the therapeutically effective amount of the compound is 800 mg. In some embodiments, the disclosure is directed to a method of treating cancer, such as breast cancer, in a subject, wherein the patient, prior to administering a compound disclosed herein, has undergone at least one other cancer therapy (e.g., systemic therapy, endocrine therapy). In some embodiments, the at least one other cancer therapy comprises treatment with a CDK4/6 inhibitor, aromatase inhibitor, SERD, covalent antagonist (SERCA), or an ER chimeric degrader. In some embodiments, the at least one other cancer therapy is administering a SERD, e.g., wherein the SERD is fulvestrant. In some embodiments, the at least one other cancer therapy is administering a CDK4/6 inhibitor, e.g., wherein the CDK4/6 inhibitor is abemaciclib, palbociclib, or ribociclib.

In some embodiments, the compounds disclosed herein may be administered with at least one additional anti-cancer agent, such as abemaciclib, palbociclib, or ribociclib.

In some embodiments, the disclosure is directed to a method of treating cancer in a patient in need thereof, comprising administering to the patient a therapeutically effective amount of a compound or a mixture of compounds, wherein the compound is represented by Formula 1 or is a pharmaceutically acceptable salt thereof:

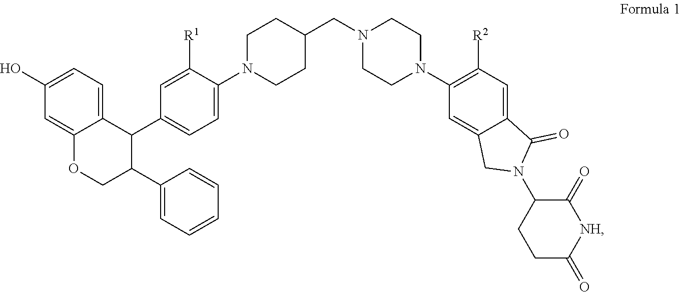

wherein the cancer is breast cancer, lung cancer, prostate cancer, stomach cancer, pancreatic cancer, ovarian cancer, melanoma, endocrine cancer, uterine cancer, testicular cancer, or bladder cancer. In some embodiments, the cancer is breast cancer, lung cancer, prostate cancer, pancreatic cancer, or ovarian cancer. In some embodiments, the cancer is breast cancer, lung cancer, or prostate cancer. In some embodiments, the cancer is breast cancer.

### BRIEF DESCRIPTION OF THE DRAWINGS

FIG. 1 depicts a Phase 1 dose-escalation study investigating compound (1-g) administered orally (PO) as a single agent.

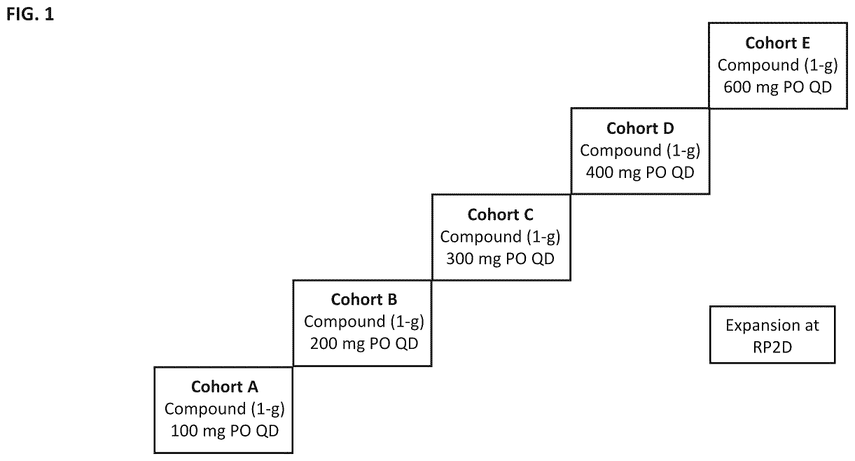

FIG. 2 depicts the treatment plan for a Phase 2 study evaluating two dose levels of compound (1-g) in patients with advanced or metastatic breast cancer.

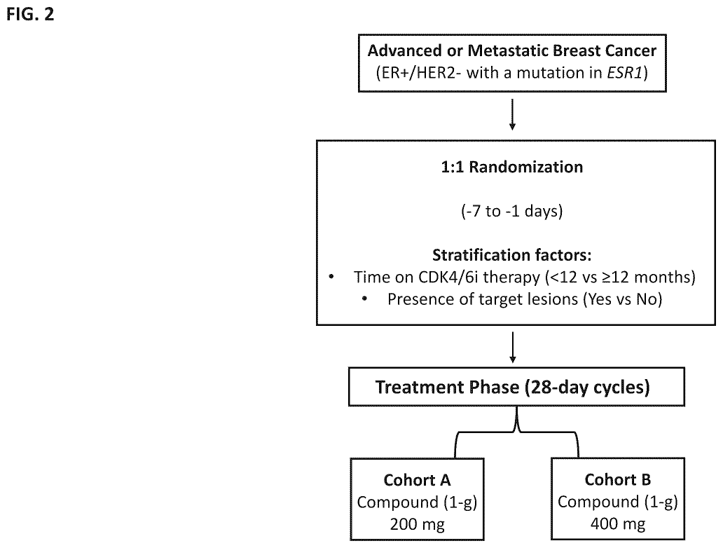

FIG. 3A depicts the growth inhibitory effects demonstrated by compound (1-g) in a luminescence-based MCF7 cell proliferation assay.

FIG. 3B depicts the growth inhibitory effects demonstrated by compound (1-h) in a luminescence-based MCF7 cell proliferation assay.

FIG. 4A depicts the plasma concentration of compound (1-g) (ng/ml) in patient plasma samples over the course of 24 hours. Patients were orally administered 100 mg, 200 mg, and 300 mg doses of compound (1-g) once a day.

FIG. 4B depicts the plasma concentration of compound (1-h) (ng/mL) in patient samples over the course of 24 hours. Patients were orally administered 100 mg, 200 mg, and 300 mg doses of compound (1-g) once a day and compound (1-h) (an epimer of (1-g)) was detected in the patient plasma samples.

FIG. 4C depicts the plasma concentration of compound (1-g) (ng/mL) in patient samples over the course of 24 hours. Patients were orally administered 100 mg, 200 mg, 300 mg, and 400 mg doses of compound (1-g) once a day.

FIG. 4D depicts the plasma concentration of compound (1-g) (ng/ml) in patient samples over the course of 24 hours post-dosing on day 15. Patients were orally administered 100 mg, 200 mg, 300 mg, 400 mg, and 600 mg doses of compound (1-g) once a day.

FIG. 5 depicts the treatment duration in weeks for five cohorts that were administered 100 mg, 200 mg, 300 mg, 400 mg, and 600 mg of compound (1-g). Prior medications administered included CDKi, fulvestrant, or SERD or SERCA. “CDKi” stands for CDK4/6 inhibitor; “Ful” stands for fulvestrant; “SERD” stands for selective estrogen receptor degrader (including selective estrogen receptor covalent antagonist); “ER mut” stands for estrogen receptor 1 mutation at baseline (variant allele frequency ≥1%); “P” stands for pending; “PD” stands for progressive disease; “PR” stands for partial response; “SD” stands for stable disease; “TL” stands for target lesion; “SERCA” stands for selective estrogen receptor covalent antagonist; “N” stands for number of evaluable participants. The duration of treatment for all participants (n=37) who received at least 1 dose is shown at the cutoff date. Each bar represents the time on study for each participant.

FIG. 6 depicts the change in solid tumor lesions from baseline in participants with evaluable target lesions (n=26) with ESR1 mutation and wild type (WT) ER. “ESR1” stands for estrogen receptor 1 gene. Changes in tumor size at the cutoff date for each of 26 evaluable participants are shown. Each bar represents the response observed for each participant. Striped bars are participants with a tumor bearing the ESR1 mutation.

### DETAILED DESCRIPTION

### Definitions

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The abbreviations used herein have their conventional meaning without the chemical and biological arts. The chemical structures and formulae set forth herein are constructed according to the standard rules of chemical valency known in the chemical arts.

The terms “halo” or “halogen” as used herein refer to —F, —Cl, —Br, and/or —I.

“H” refers to hydrogen.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

The compounds, tautomers, solvates, or pharmaceutically acceptable salts of the disclosure may contain, in some embodiments, a meso moiety, be a meso compound, or have meso isomerism.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

“Stereoisomer” or “optical isomer” means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of(S)- and (R)-isomers. In other embodiments, provided herein is a mixture of compounds wherein individual compounds of the mixture exist predominately in an(S)- or (R)-isomeric configuration. For example, the compound mixture has an(S)-enantiomeric excess of greater than 55%, 60%, 65%, 70%, 75%, 80%, 85%, 90%, 95%, 96%, 97%, 98%, 99%, 99.5%, or more. In other embodiments, the compound mixture has an(S)-enantiomeric excess of greater than 55% to 99.5%, greater than 60% to 99.5%, greater than 65% to 99.5%, greater than 70% to 99.5%, greater than 75% to 99.5%, greater than 80% to 99.5%, greater than 85% to 99.5%, greater than 90% to 99.5%, greater than 95% to 99.5%, greater than 96% to 99.5%, greater than 97% to 99.5%, greater than 98% to greater than 99.5%, greater than 99% to 99.5%, or more. In other embodiments, the compound mixture has an (R)-enantiomeric purity of greater than 55%, 60%, 65%, 70%, 75%, 80%, 85%, 90%, 95%, 96%, 97%, 98%, 99%, 99.5% or more. In some other embodiments, the compound mixture has an (R)-enantiomeric excess of greater than 55% to 99.5%, greater than 60% to 99.5%, greater than 65% to 99.5%, greater than 70% to 99.5%, greater than 75% to 99.5%, greater than 80% to 99.5%, greater than 85% to 99.5%, greater than 90% to 99.5%, greater than 95% to 99.5%, greater than 96% to 99.5%, greater than 97% to 99.5%, greater than 98% to greater than 99.5%, greater than 99% to 99.5% or more.

The term “purity” as used herein refers to the amount of a compounds disclosed herein (e.g., compounds of Formula 1) based on High performance liquid chromatography (HPLC). Purity is based on the “organic” purity of the compound. Purity does not include a measure of any amount of water, solvent, metal, inorganic salt, etc. In one embodiment, the purity of, for example, compound (1-g) is compared to the purity of the reference standard by comparing the area under the peak. In one embodiment, the known standard for purity is a compound (1-g) reference standard. In some embodiments, compound (1-g) has a purity of greater than 95%. In some embodiments, compound (1-g) has a purity of greater than 98%. For example, the purity of compound (1-g) is 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%. For example, the purity of compound (1-g) is 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%. For example, the purity of compound (1-g) is 98.0%, 98.5%, 99.0%, 99.5%, 99.6%, 99.7%, 99.8%, or 99.9%. For example, the purity of compound (1-g) is 98.5%, 99.0%, or 99.5%. In some embodiments, the present disclosure relates to compound (1-g) having a purity greater than 98%. In one embodiment, the purity is determined by HPLC. In another embodiment, the present invention relates to compound (1-g), or a pharmaceutically acceptable salt thereof. In one embodiment, the purity is greater than 98.5%. In one embodiment, the purity is greater than 99.0%. In one embodiment, the purity is greater than 99.5%. In one embodiment, the impurity is compound (1-h).

Individual stereoisomers of compounds of the present disclosure can be prepared synthetically from commercially available starting materials that contain asymmetric or stereogenic/chiral centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically composition” as used herein refers to a composition comprising at least one compound as disclosed herein formulated together with one or more pharmaceutically acceptable carriers.

Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

As used herein, “cancer” refers to diseases, disorders, and conditions that involve abnormal cell growth with the potential to invade or spread to other parts of the body. Exemplary cancers include, but are not limited to, breast cancer, lung cancer, ovarian cancer, endometrial cancer, prostate cancer, and esophageal cancer.

“Metastatic breast cancer (or “metastases”) refers to breast cancer that has spread beyond the breast and nearby lymph nodes to other parts of the body, e.g., bones, liver, lungs, brain. (https://www.cancer.org/cancer/breast-cancer.)

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon, ape, or rhesus.

In some embodiments, the subject is a human.

In some embodiments, the subject is a human who has been diagnosed with breast cancer.

In some embodiments, the subject is a human who has been diagnosed with metastatic breast cancer. In some embodiments, the subject is a human who has been diagnosed with ER-positive/HER2-negative breast cancer.

In some embodiments, the subject is a human who has been diagnosed with metastatic, ER-positive, HER2-negative breast cancer.

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the term “mixture” when used in the context of a “mixture” of two compounds means that both compounds are present in the mixture. By way of example, if a mixture of compound A and compound B has a relative amount of compound A and compound B greater than 99:1, such a mixture cannot have a ratio of 100:0 as this would not be a “mixture” as defined herein. Similarly, if such an exemplary mixture comprises less than 1% by weight of compound B, such a mixture cannot have 0% by weight of compound B as this would not be a “mixture” as defined herein. And if such an exemplary mixture has a weight percent of compound A greater than 99% and the remainder is compound B, such a mixture cannot have 100% by weight of compound A as this would not be a “mixture” as defined herein.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As used herein, the phrase “therapeutic agent” refers to any agent that has a therapeutic effect and/or elicits a desired biological and/or pharmacological effect, when administered to a subject.

As used herein, the term “therapeutically effective amount” refers to an amount of a therapeutic agent that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent effective to treat, ameliorate, or prevent a desired disease or condition, or to exhibit a detectable therapeutic or preventive effect, such as by ameliorating symptoms associated with the disease, preventing or delaying the onset of the disease or condition, and/or also lessening the severity or frequency of symptoms of the disease or condition. A therapeutically effective amount is commonly administered in a dosing regimen that may comprise multiple doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As is used herein, “ER” refers to human estrogen receptor alpha (ERα) encoded by the human ESR1 gene. Somatic ER tumor mutations are observed with increased frequency in patients having breast cancer that has acquired resistance to endocrine therapies (see Toy et al. (2013) Nature Genetics 45:1439-1445; Merenbakh-Lamin et al. (2013) Cancer Research 73:6856-6864; Robinson et al. (2013) Nature Genetics 45:1446-1451; Li et al. (2013) Cell Reports 4:1116-1130). Somatic ER mutations occur frequently in the ER ligand binding domain, which is the functional domain of human ER that forms a hydrophobic pocket for binding the ER hormone ligand (e.g., estrogen) (Hamadch et al. (2018) Cancer Treat Rev 70:47-55; Jeselsohn, et al. (2015) Nat Rev Clin Oncol 12:573-583). Moreover, it has been demonstrated that somatic ER tumor mutations in the ER ligand binding domain are acquired in response to selective pressure of endocrine therapies that create a low-estrogen environment (e.g., aromatase inhibitors) (see Jeselsohn et al. (2014) Clinical Cancer Research 20:1757-1767; Schiavon, et al. (2015) Sci Transl Med 7: 313ral82). Without wishing to be bound by theory, mutations in the ER ligand binding domain result in decreased ligand specificity, thereby enabling ER to function independently of estrogen. Such ER tumor mutations provide tumor cells with the capability to proliferate in estrogen-depleted environments, and thus are selected for in response to endocrine therapies that block or reduce estrogen levels.

As understood by the skilled artisan, ER is a polypeptide that is 595 amino acid residues in length and comprises three functional domains: the N-terminal transcriptional regulation domain, the DNA-binding domain, and the ligand binding domain (see Kumar, et al. (2011) J Amino Acids Article ID 812540). The DNA-binding domain is linked to the ligand-binding domain via a hinge. A suitable reference sequence for the ER is set forth by SEQ ID NO: 1 and identified in the UniProt database as P03372 (ESR1_HUMAN).

As is used herein, “ER+” (estrogen receptor positive), refers to breast cancer cells that have a receptor protein that binds the hormone estrogen. Cancer cells that are ER+ may need estrogen to grow and may stop growing or die when treated with substances that block the binding and actions of estrogen, (https://www.cancer.gov/publications/dictionaries/cancer-terms/def/44404.)

As is used herein, “ESR1” refers to a gene that makes one of the two main types of estrogen receptor (ER) proteins. These proteins bind to and receive signals from the hormone estrogen. They are found inside the cells of female reproductive tissue, breast tissue, and many other types of tissue, and some cancer cells. Once activated by estrogen, ER proteins control the activity of certain genes that play an important role in cell growth and metabolism, sexual development, pregnancy, and other reproductive functions. Mutations in the ESR1 gene can cause the ER protein to be too active or found in higher-than-normal amounts on or in some types of cancer cells, such as breast or endometrial cancer cells. This may cause cancer cells to grow and spread. It may also make cancer cells to not respond to certain anticancer drugs. The protein made by the ESR1 gene is a type of nuclear hormone receptor and a type of transcription factor, (https://www.cancer.gov/publications/dictionaries/cancer-terms/def/esr1-gene).

As used herein, “HER2-” (human epidermal growth factor receptor 2) refers to breast cancer cells that does not have a large amount of a protein called HER2 on their surface. In normal cells, HER2 helps to control cell growth. Cancer cells that are HER2− may grow more slowly and are less likely to recur or spread to other parts of the body than cancer cells that have a large amount of HER2 on their surface. (https://www.cancer.gov/publications/dictionaries/cancerterms/def/her2-negative.)

As used herein, the “N-terminal transcriptional regulation domain” refers to a contiguous stretch of amino acid residues extending from amino acid residue 1 to about amino acid residue 180 of the ER (e.g., amino acid residues 1-180 of SEQ ID NO: 1). In some embodiments, the “N-terminal transcriptional regulation domain” refers to a contiguous stretch of amino acid residues extending from amino acid residue 1 to amino acid residue 180 of the ER (e.g., amino acid residues 1-180 of SEQ ID NO: 1).

As used herein, the “DNA-binding domain” refers to a contiguous stretch of amino acid residues extending from about amino acid residue 181 to about amino acid residue 263 of the ER (e.g., amino acid residues 181-263 of SEQ ID NO: 1). In some embodiments, the “DNA-binding domain” refers to a contiguous stretch of amino acid residues extending from amino acid residue 181 to amino acid residue 263 of the ER (e.g., amino acid residues 181-263 of SEQ ID NO: 1).

As used herein, the “hinge” refers to a contiguous stretch of amino acid residues extending from about amino acid residue 264 to about amino acid residue 302 of the ER (e.g., amino acid residues 264-302 of SEQ ID NO: 1). In some embodiments, the “hinge” refers to a contiguous stretch of amino acid residues extending from amino acid residue 264 to amino acid residue 302 of the ER (e.g., amino acid residues 264-302 of SEQ ID NO: 1).

As used herein, the “ligand binding domain” refers to a contiguous stretch of amino acid residues extending from about amino acid residue 303 to about amino acid residue 552 (e.g., amino acid residues 303-552 of SEQ ID NO: 1). In some embodiments, the “ligand binding domain” refers to a contiguous stretch of amino acid residues extending from amino acid residue 303 to amino acid residue 552 (e.g., amino acid residues 303-552 of SEQ ID NO: 1).

As used herein, a subject is “in need of” a treatment if such subject would benefit biologically, medically or in quality of life from such treatment.

“Administration” refers to introducing an agent, such as a compound of Formula 1 or a pharmaceutically acceptable salt thereof, into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

As used herein, “anti-cancer agent” is used to describe an anti-cancer agent, or a therapeutic agent administered concurrently with an anti-cancer agent (e.g., SERDs, CDK4/6 inhibitors), with which may be co-administered and/or co-formulated with a compound of Formula (I) to treat cancer, and the side effects associated with the cancer treatment.

As used herein, “Cₘₐₓ” refers to the observed maximum (peak) plasma concentration of a specified compound in the subject after administration of a dose of that compound to the subject.

As used herein, “AUC” refers to the total area under the plasma concentration-time curve, which is a measure of exposure to a compound of interest (e.g., compounds of Formula 1), and is the integral of the concentration-time curve after a single dose or at steady state. AUC is expressed in units of ng*hr/mL (ng×hr/mL).

As used herein, “AUCₜₐᵤ” refers to the AUC from 0 hours to the end of a dosing interval.

As used herein, “Tₘₐₓ” refers to time to maximum concentration.

“Oral dosage form” as used herein refers to a pharmaceutical drug product that contains a specified amount (dose) of a compound of Formula 1 as the active ingredient, or a pharmaceutically acceptable salt and/or solvate thereof, and inactive components (excipients), formulated into a particular configuration that is suitable for oral administration and drug delivery, such as a tablet, capsule, or liquid oral formulation. In some embodiments, the compositions are in the form of a tablet that can be scored.

As used herein, the term “carrier” encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

“Excipient” refers to an inactive component of a pharmaceutical composition, formulated into a particular configuration that is suitable for oral administration and drug delivery, such as a tablet, capsule or liquid oral formulation.

As used herein, the terms “fasted condition” or “fasted state” are used to describe a subject means the subject has not eaten for at least 4 hours before a time point of interest, such as the time of administering a compound disclosed herein.

“Fed condition” or “fed state” as used to describe a subject herein means the subject has eaten less than 4 hours before a time point of interest, such as the time of administering a compound disclosed herein.

As used herein, the term “CDK4/6 inhibitor” refers to a compound that inhibits the enzymes in humans referred to as cyclin-dependent kinases (CDK) 4 and 6. Examples of a CDK4/6 inhibitor include, without limitation, SHR6390, trilaciclib, lerociclib, AT7519M, dinaciclib, ribociclib, abemaciclib, palbociclib, or any pharmaceutically acceptable salt thereof. In some embodiments, the CDK4/6 inhibitor is palbociclib or a pharmaceutically acceptable salt thereof.

“Tumor” refers to an abnormal new growth of tissue that possesses no physiological function and arises from uncontrolled usually rapid cellular proliferation.

“Tumor mutation” refers to genetic mutations in key regulatory genes that alter the behavior of cells and can potentially lead to the unregulated growth seen in cancer. Tumors with high mutational burden have a large number of genetic mutations within their cells, which can be acquired as a result of exposure to harmful agents such as ultraviolet light or certain chemicals in tobacco.

As used herein, a gene status of “wild-type” (WT) is meant to indicate normal or appropriate expression of the gene and normal function of the encoded protein. In contrast, mutant status is meant to indicate expression of a protein with altered function, consistent with the known roles of mutant ERα genes in cancer (as described herein). Any number of genetic or epigenetic alterations, including but not limited to mutation, amplification, deletion, genomic rearrangement, or changes in methylation profile, may result in a mutant status. However, if such alterations nevertheless result in appropriate expression of the normal protein, or a functionally equivalent variant, then the gene status is regarded as wild-type. Examples of variants that typically would not result in a functional mutant gene status include synonymous coding variants and common polymorphisms (synonymous or non-synonymous). Gene status can be assessed by a functional assay, or it may be inferred from the nature of detected deviations from a reference sequence.

As used herein, the term “del” denotes an in-frame deletion of the amino acid residue(s) relative to wild type. For example, “V422del” indicates that a mutant in which the valine at position 422 in the wild-type ER protein has been deleted.

As used herein, an underscore between the notation of two amino acids indicates that the sequence of residues, inclusive of both endpoints, has been altered. For example, “L536_D538>P” indicates a mutant arising from an in-frame deletion resulting in the amino acid residues beginning with lysine at position 536 and ending at aspartic acid at position 538 having been replaced by a single proline.

“Complete response”, or “CR” as described herein refers to the disappearance of all target and non-target lesions since baseline. Any pathological lymph nodes must have a reduction in short axis to <10 mm.

“Dose limiting toxicity”, or “DLT” as used herein refers to any of the toxicities if considered at least possibly related to study treatment. The toxicities may be attributable to the disease or disease-related process under investigation. The toxicities may be attributable to at least one of the hematologic or non-hematologic criteria.

“Progressive disease”, or “PD” as described herein refers to the at least 20% increase in the sum of diameters of target lesions of a tumor, taking as reference the smallest sum on study or nadir (this includes the baseline sum if that is the smallest on study). In addition to the relative increase of 20%, the sum must also demonstrate an absolute increase of at least 5 mm. Unequivocal progression of existing non-target lesions from baseline and the appearance of one or more new lesions is also considered as PD.

“Partial response”, or “PR” as described herein refers to the at least 30% decrease in the sum of the diameters of target lesions of a tumor, taking as reference the baseline sum of diameters.

“Stable disease”, or “SD” as described herein refers to an objective tumor response displaying neither sufficient shrinkage to qualify for PR nor sufficient increase to qualify for PD.

“Systemic therapy” as described herein refers to a type of cancer treatment that targets the entire body. Systemic therapy may include chemotherapy, hormone therapy, targeted therapy, immune therapy, and systemic radiation therapy. Systemic therapy may be administered as an injection, infusion, or oral medication.

“RECIST Version 1.1” as described herein refers to Response Evaluation Criteria in Solid Tumors Version 1.1 as described in detail in Tables 1 and 2 of this application. See Eisenhauer et al. 2009 European Journal of Cancer 45:228-247; Nishino M., Revised RECIST Guideline Version 1.1: What Oncologists Want to Know and What Radiologists Need to Know, vol 195, issue 2, https://doi.org/10.2214/AJR.09.4110.

“ORR” as described herein refers to objective response rate, defined as proportion of patients with confirmed complete response or partial response according to the RECIST Version 1.1 criteria.

“CBR” as described herein refers to clinical benefit rate, defined as proportion of patients with confirmed complete response, partial response, or stable disease for ≥24 weeks according to the RECIST Version 1.1 criteria.

“DOR” as described herein refers to duration of response, defined as the time between first documentation of a response (complete response or partial response) and first evidence of PD according to RECIST v1.1 or death due to any cause.

“DCR” as described herein refers to disease control rate, defined as the proportion of patients with a best overall response of complete response, partial response, or stable disease.

“PFS” as described herein refers to progression-free survival, defined as the time from the first day of study drug administration until objective disease progression as defined by the RECIST Version 1.1 criteria, or death on study, whichever occurs first.

### Compounds

In some embodiments, the compounds disclosed herein possess one or more stereocenters and each center may exist in the R or S configuration. The compounds presented herein include all diastereomeric, enantiomeric, and epimeric forms as well as the corresponding mixtures thereof. In some embodiments, the compounds disclosed herein are epimers (e.g., compounds (1-e) and (1-f); compounds (1-g) and (1-h)). In some embodiments, the compounds disclosed herein may show antagonism of ER dependent transcription but may not degrade the ER. In some embodiments, some of the compounds disclosed herein show both antagonism of ER dependent transcription and degrade ER.

In some embodiments, the present disclosure provides a compound, wherein the compound is of the following structure:

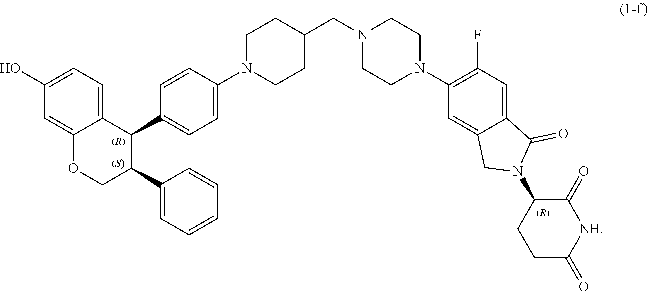

In some embodiments, the present disclosure provides a compound, wherein the compound is of the following structure:

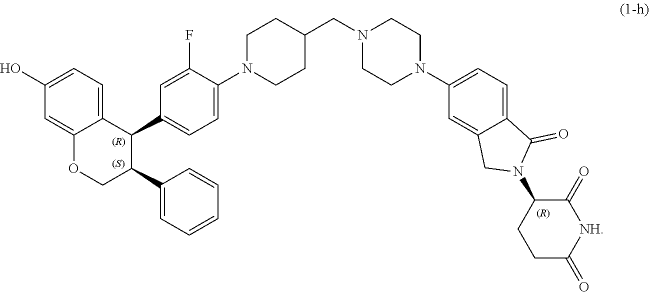

In some embodiments, the present disclosure provides a compound, wherein the compound is of the following structure:

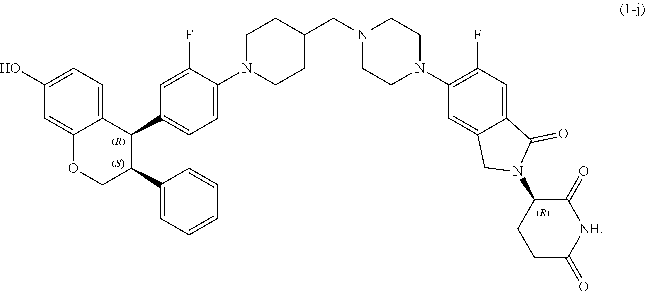

In some embodiments, the present disclosure provides a compound, wherein the compound is of the following structure:

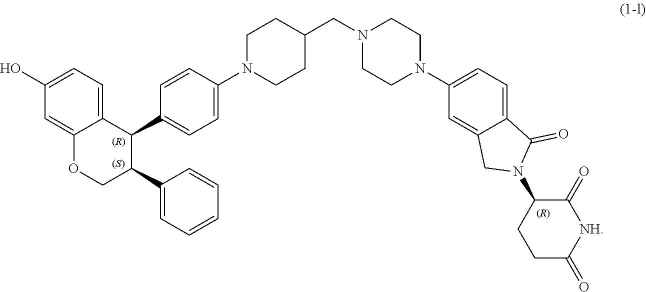

In some embodiments, at least two compounds of Formula 1, or a pharmaceutically acceptable salt thereof, are present as a mixture of the two compounds, such as a mixture of two epimeric compounds. In some embodiments, the mixture comprises varying amounts (e.g., the relative amounts of epimeric compounds (1-e) and (1-f) are different, the relative amounts of epimeric compounds (1-g) and (1-h) are different, the relative amounts of epimeric compounds (1-i) and (1-j) are different, the relative amounts of epimeric compounds (1-k) and (1-1) are different) of the two compounds.

In some embodiments, the relative amounts of compounds (1-e) and (1-f) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the relative amounts of compounds (1-e) and (1-f) in the mixture are greater than 95:5, such as greater than 98:2 or 99:1. In some embodiments, the relative amounts of compounds (1-e) and (1-f) are greater than 90:10. In some embodiments, the relative amounts of compounds (1-e) and (1-f) are greater than 85:15. In some embodiments, the relative amounts of compounds (1-e) and (1-f) are greater than 80:20.

In some embodiments, the relative amounts of compounds (1-g) and (1-h) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the relative amounts of compounds (1-g) and (1-h) in the mixture are greater than 95:5, such as greater than 98:2 or 99:1. In some embodiments, the relative amounts of compounds (1-g) and (1-h) are greater than 90:10. In some embodiments, the relative amounts of compounds (1-g) and (1-h) are greater than 85:15. In some embodiments, the relative amounts of compounds (1-g) and (1-h) are greater than 80:20.

In some embodiments, the relative amounts of compounds (1-i) and (1-j) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the relative amounts of compounds (1-i) and (1-j) in the mixture are greater than 95:5, such as greater than 98:2 or 99:1. In some embodiments, the relative amounts of compounds (1-i) and (1-j) are greater than 90:10. In some embodiments, the relative amounts of compounds (1-i) and (1-j) are greater than 85:15. In some embodiments, the relative amounts of compounds (1-i) and (1-j) are greater than 80:20.

In some embodiments, the relative amounts of compounds (1-k) and (1-1) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the relative amounts of compounds (1-k) and (1-1) in the mixture are greater than 95:5, such as greater than 98:2 or 99:1. In some embodiments, the relative amounts of compounds (1-k) and (1-1) are greater than 90:10. In some embodiments, the relative amounts of compounds (1-k) and (1-1) are greater than 85:15. In some embodiments, the relative amounts of compounds (1-k) and (1-1) are greater than 80:20.

In some embodiments, the compound of Formula 1 consists of a mixture of two epimeric compounds, wherein the mixture comprises less than 5% by weight of one or more of a compound selected from

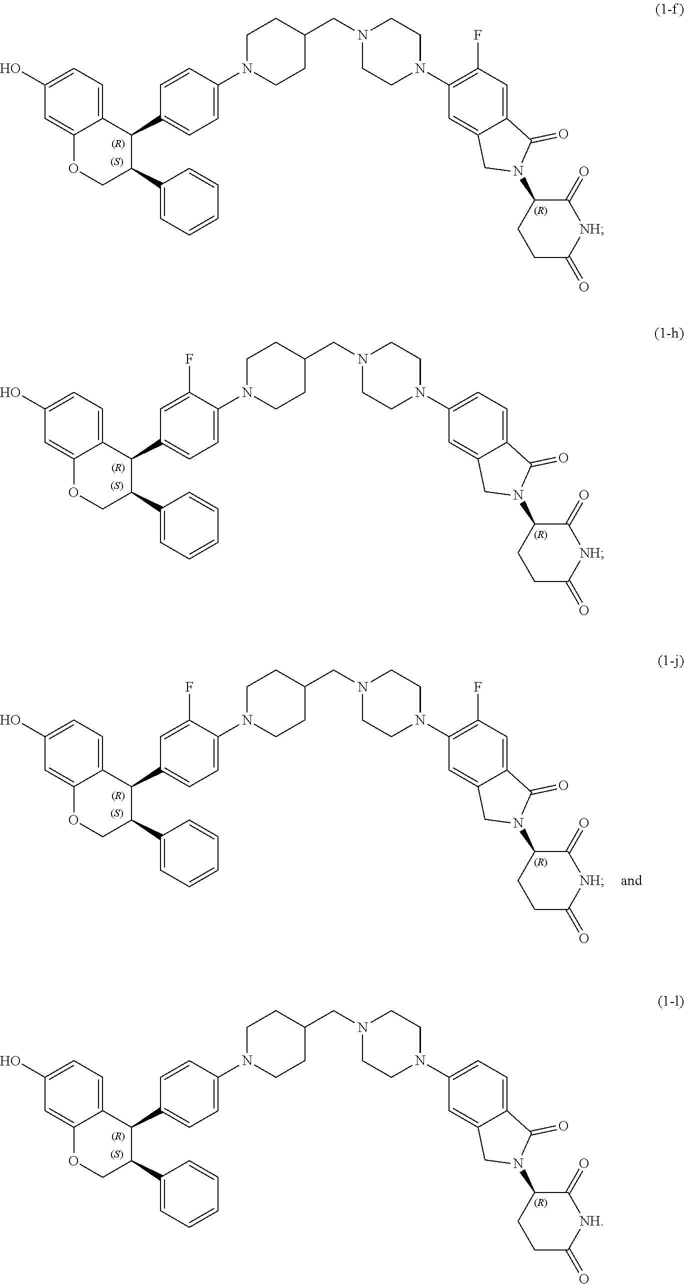

In some embodiments, the compound of Formula 1 consists of a mixture of two epimeric compounds, wherein the mixture comprises less than 10%, 5%, 4%, 3%, 2%, 1%, or 0.5% by weight of compound (1-f), (1-h), (1-j), or (1-1). In some embodiments, the mixture comprises less than 1% of compound (1-f). In some embodiments, the mixture comprises less than 1% of compound (1-h). In some embodiments, the mixture comprises less than 1% of compound (1-j). In some embodiments, the mixture comprises less than 1% of compound (1-1).

In some embodiments, the compound of Formula 1 consists of a mixture of compounds (1-e) and (1-f), for example, wherein the mixture comprises less than 10%, 5%, 4%, 3%, 2%, 1° C., or 0.5% by weight of compound (1-f). In some embodiments, the compound of Formula 1 consists of a mixture of compounds (1-e) and (1-f), wherein the mixture comprises less than 10% of compound (1-f). In some embodiments, the compound of Formula I consists of a mixture of compounds (1-e) and (1-f), wherein the mixture comprises less than 5% of compound (1-f). In some embodiments, the compound of Formula I consists of a mixture of compounds (1-e) and (I-f), wherein the mixture comprises less than 2% of compound (1-f). In some embodiments, the compound of Formula 1 consists of a mixture of compounds (1-e) and (1-0), wherein the mixture comprises less than 1% of compound (1-f). In some embodiments, the relative amounts of compounds (1-e) and (1-f) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the compound of Formula I consists of a mixture of compounds (1-g) and (1-h), for example, wherein the mixture comprises less than 10%, 5%, 4%, 3%. 2%, 19%, or 0.5% by weight of compound (1-h). In some embodiments, the compound of Formula 1 consists of a mixture of compounds (1-g) and (1-h), wherein the mixture comprises less than 10% of compound (1-h). In some embodiments, the compound of Formula 1 consists of a mixture of compounds (1-g) and (1-h), wherein the mixture comprises less than 5% of compound (1-h). In some embodiments, the compound of Formula 1 consists of a mixture of compounds (1-g) and (1-h), wherein the mixture comprises less than 2% of compound (1-h). In some embodiments, the compound of Formula I consists of a mixture of compounds (1-g) and (1-h), wherein the mixture comprises less than 1% of compound (1-h). In some embodiments, the relative amounts of compounds (1-g) and (1-h) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the compound of Formula 1 consists of a mixture of compounds (1-i) and (1-j), for example, wherein the mixture comprises less than 10%, 5%, 4%, 3%, 2%, 1%, or 0.5% by weight of compound (1-j). In some embodiments, the compound of Formula 1 consists of a mixture of compounds (1-i) and (1-j), wherein the mixture comprises less than 10% of compound (1-j). In some embodiments, the compound of Formula 1 consists of a mixture of compounds (1-i) and (1-j), wherein the mixture comprises less than 5% of compound (1-j). In some embodiments, the compound of Formula 1 consists of a mixture of compounds (1-i) and (1-j), wherein the mixture comprises less than 2% of compound (1-j). In some embodiments, the compound of Formula 1 consists of a mixture of compounds (1-i) and (1-j), wherein the mixture comprises less than 1% of compound (1-j). In some embodiments, the relative amounts of compounds (1-i) and (1-j) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the compound of Formula 1 consists of a mixture of compounds (1-k) and (1-1), for example, wherein the mixture comprises less than 10%, 5%, 4%, 3%, 2%, 1%, or 0.5% by weight of compound (1-1). In some embodiments, the compound of Formula 1 consists of a mixture of compounds (1-k) and (1-1), wherein the mixture comprises less than 10% of compound (1-1). In some embodiments, the compound of Formula 1 consists of a mixture of compounds (1-k) and (1-1), wherein the mixture comprises less than 5% of compound (1-1). In some embodiments, the compound of Formula I consists of a mixture of compounds (1-k) and (1-1), wherein the mixture comprises less than 2% of compound (1-1). In some embodiments, the compound of Formula I consists of a mixture of compounds (1-k) and (1-1), wherein the mixture comprises less than 1% of compound (1-1). In some embodiments, the relative amounts of compounds (1-k) and (1-1) in the mixture are from 99:1 to 70:30, such as 95:5 to 80:20, 95:5 to 85:15, 95:5 to 90:10, 90:10 to 80:20, 90:10 to 85:15, or 85:15 to 80:20.

In some embodiments, the present disclosure is directed to a mixture of the following compounds

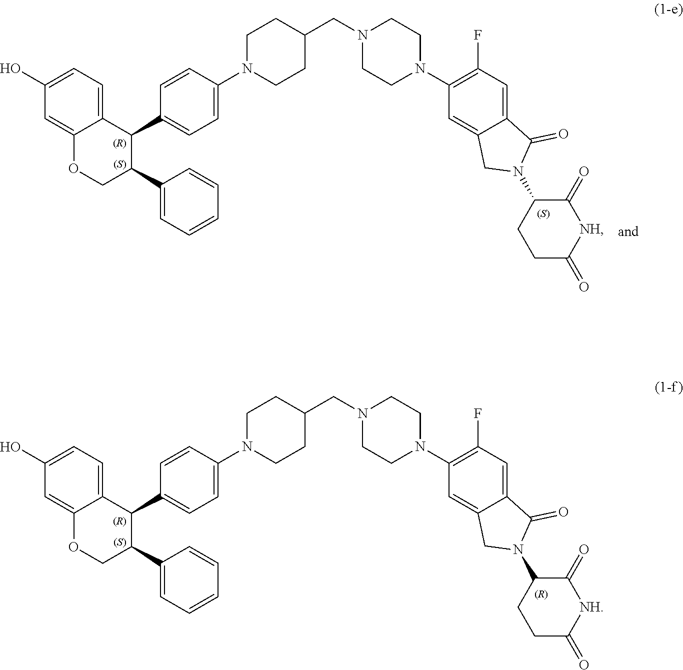

wherein the mixture comprises less than 10%, 5%, 4%, 3%, 2%, or 1% by weight of compound (1-f), particularly less than 1% of compound (1-f).

In some embodiments, the present disclosure is directed to a mixture of the following compounds

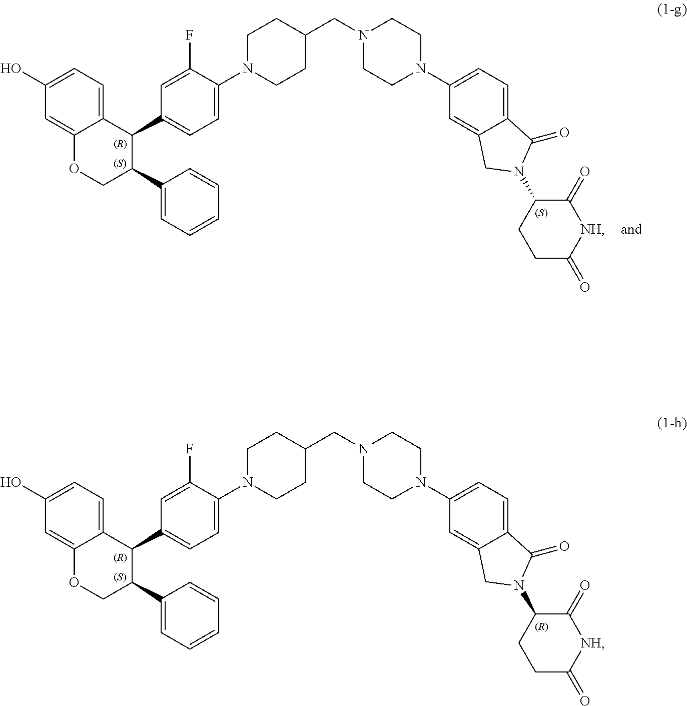

wherein the mixture comprises less than 10%, 5%, 4%, 3%, 2%, or 1% by weight of compound (1-h), particularly less than 1% of compound (1-h).

In some embodiments, the present disclosure is directed to a mixture of the following compounds

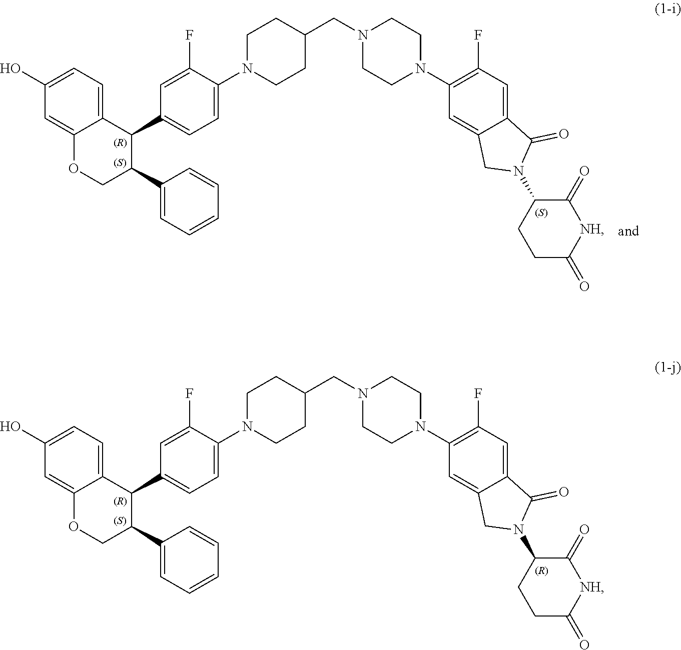

wherein the mixture comprises less than 10%, 5%, 4%, 3%, 2%, or 1% by weight of compound (1-j), particularly less than 1% of compound (1-j).

In some embodiments, the present disclosure is directed to a mixture of the following compounds

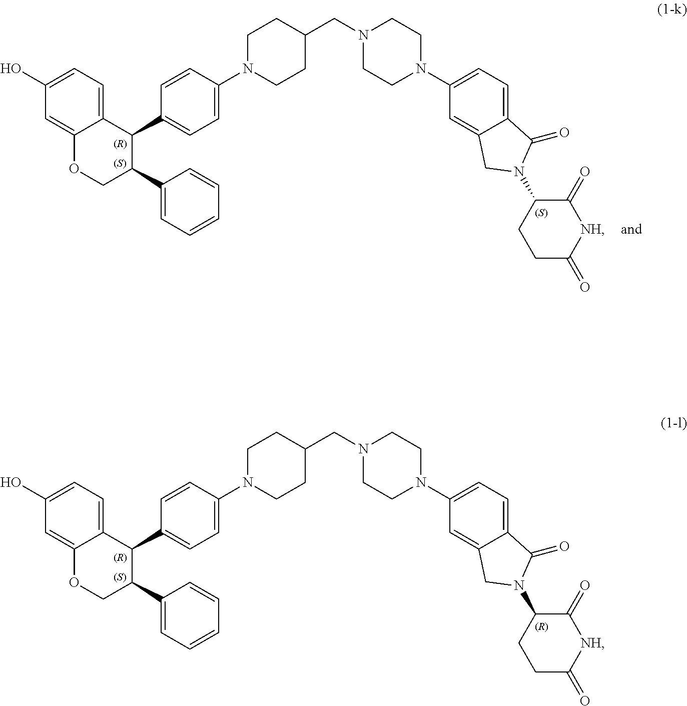

wherein the mixture comprises less than 10%, 5%, 4%, 3%, 2%, or 1% by weight of compound (1-1), particularly less than 1% of compound (1-1).

In some embodiments, the present disclosure provides a compound, wherein the compound is represented by Formula 1 or is a pharmaceutically acceptable salt thereof, and wherein the compound is selected from

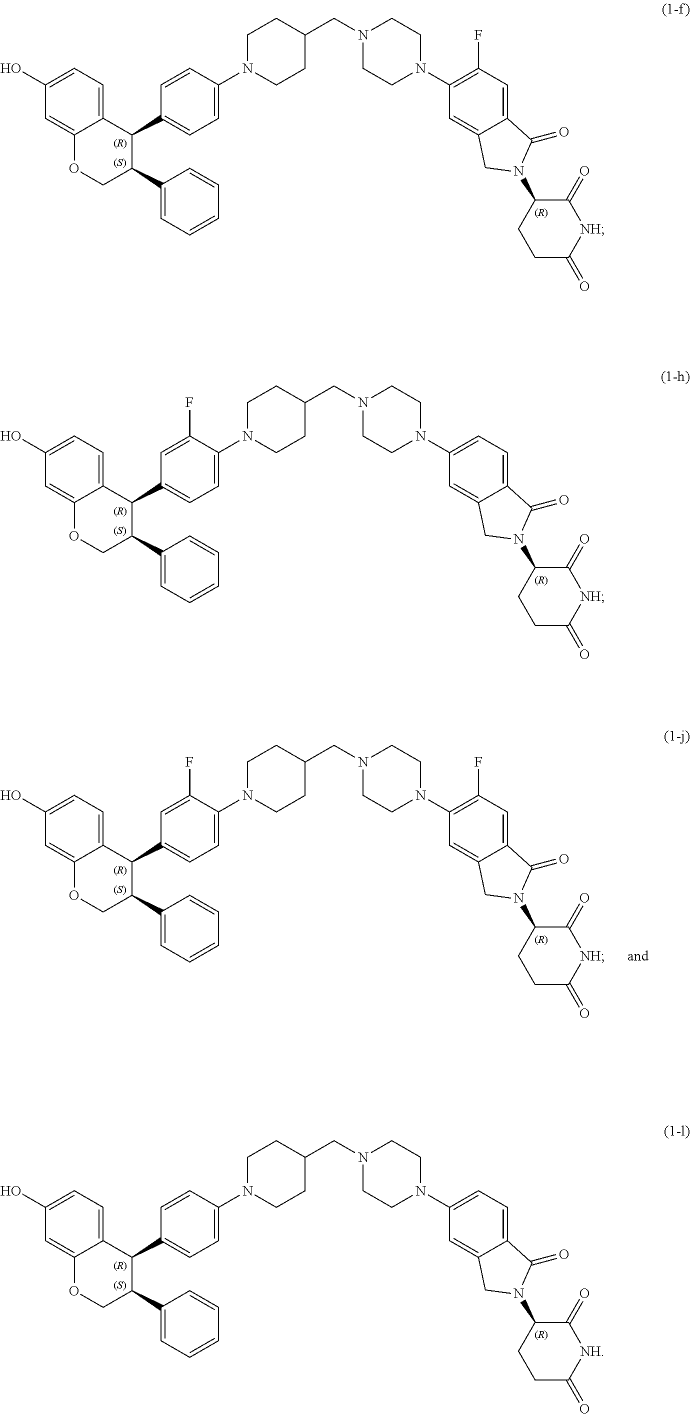

In some embodiments, the present disclosure provides a mixture of the following compounds

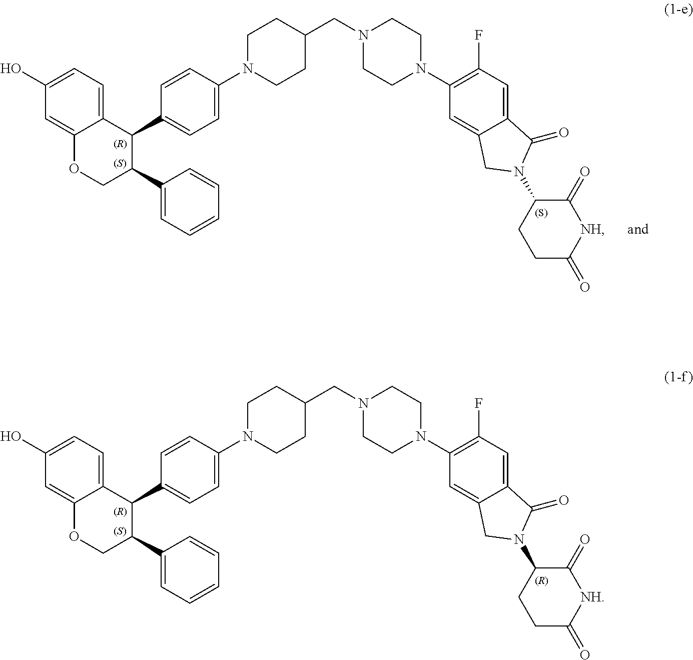

In some embodiments, the relative amounts of compound (1-e) and compound (1-f) in the mixture are greater than 95:5, such as greater than 98:2 or 99:1.

In some embodiments, the relative amounts of compounds (1-e) and (1-f) in the mixture are greater than 90:10.

In some embodiments, the relative amounts of compounds (1-e) and (1-f) in the mixture are greater than 85:15.

In some embodiments, the relative amounts of compounds (1-e) and (1-f) in the mixture are greater than 80:20.

In some embodiments, the present disclosure provides a mixture of the following compounds

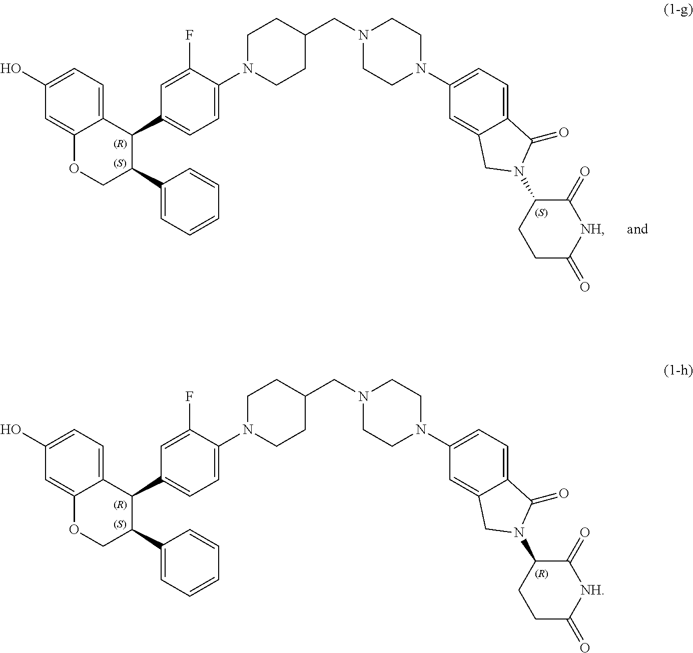

In some embodiments, the relative amounts of compound (1-g) and compound (1-h) in the mixture are greater than 95:5, such as greater than 98:2 or 99:1.

In some embodiments, the relative amounts of compounds (1-g) and (1-h) in the mixture are greater than 90:10.

In some embodiments, the relative amounts of compounds (1-g) and (1-h) in the mixture are greater than 85:15.

In some embodiments, the relative amounts of compounds (1-g) and (1-h) in the mixture are greater than 80:20.

In some embodiments, the present disclosure provides a mixture of the following compounds

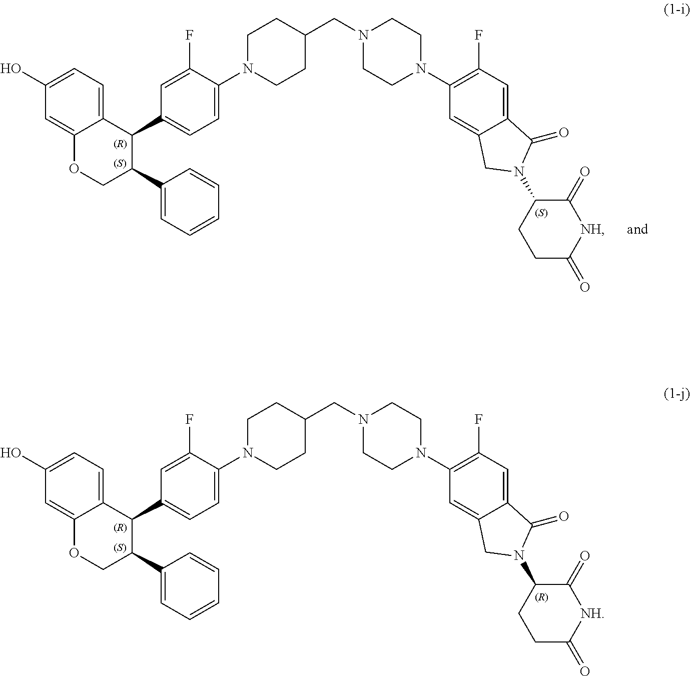

In some embodiments, the relative amounts of compound (1-i) and compound (1-j) in the mixture is greater than 95:5, such as greater than 98:2 or 99:1.

In some embodiments, the relative amounts of compounds (1-i) and (1-j) in the mixture are greater than 90:10.

In some embodiments, the relative amounts of compounds (1-i) and (1-j) in the mixture are greater than 85:15.

In some embodiments, the relative amounts of compounds (1-i) and (1-j) in the mixture are greater than 80:20.

In some embodiments, the present disclosure provides a mixture of the following compounds

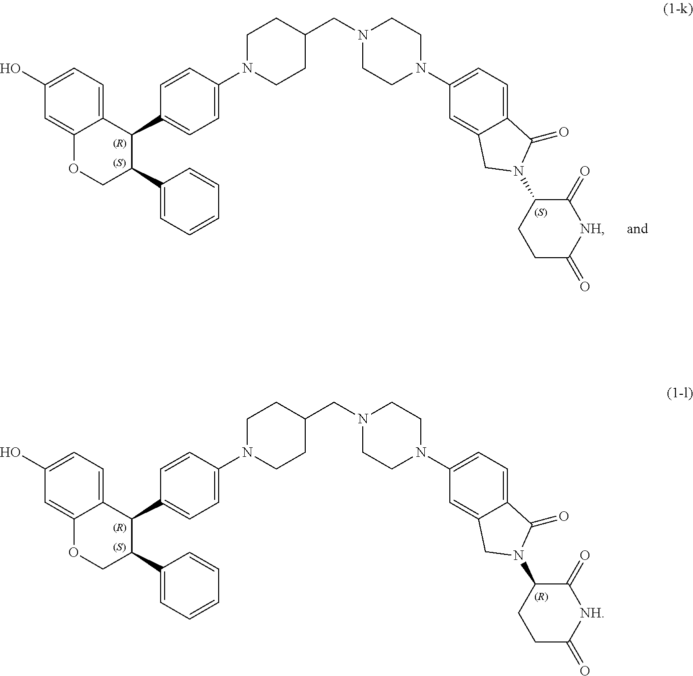

In some embodiments, the relative amounts of compound (1-k) and compound (1-1) in the mixture is greater than 95:5, such as greater than 98:2 or 99:1.

In some embodiments, the relative amounts of compounds (1-k) and (1-1) in the mixture are greater than 90:10.

In some embodiments, the relative amounts of compounds (1-k) and (1-1) in the mixture are greater than 85:15.

In some embodiments, the relative amounts of compounds (1-k) and (1-1) in the mixture are greater than 80:20.

In some embodiments, at least a particular percentage by weight of a compound disclosed herein is present in the mixture. In some embodiments, the particular weight percentages may be from 5% to 100%, such as 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40% 10% to 35%, 10% to 30% 10% to 25%, 10% to 20%, 10% to 15%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, 50% to 55%, 60% to 90%, 60% to 85%, 60% to 80%, 60% to 75%, 60% to 70%, 60% to 65%, 70% to 90%, 70% to 85%, 70% to 80%, 70% to 75%, 80% to 90%, or 80% to 85%. In some embodiments, the particular weight percentages may be 87%, 88%, 89%, 90%, 91%, 92%, 93%, 94%, 95%, 96%, 97%, 98%, 99%, 99.5%, 99.9%, or any percentage between 5% and 100%.

In some embodiments, the particular weight percentages of compound (1-e) in the mixture may be from 5% to 100%, such as 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40% 10% to 35%, 10% to 30% 10% to 25%, 10% to 20%, 10% to 15%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, 50% to 55%, 60% to 90%, 60% to 85%, 60% to 80%, 60% to 75%, 60% to 70%, 60% to 65%, 70% to 90%, 70% to 85%, 70% to 80%, 70% to 75%, 80% to 90%, or 80% to 85%, and the remainder percentage is compound (1-f). In some embodiments, the particular weight percentages of compound (1-e) in the mixture may be 87%, 88%, 89%, 90%, 91%, 92%, 93%, 94%, 95%, 96%, 97%, 98%, 99%, 99.5%, or 99.9%, and the remainder percentage is compound (1-f). In some embodiments, the particular weight percentages of compound (1-e) in the mixture may be greater than 87%, 88%, 89%, 90%, 91%, 92%, 93%, 94%, 95%, 96%, 97%, 98%, 99%, 99.5%, or 99.9%, and the remainder percentage is compound (1-f).

In some embodiments, the particular weight percentages of compound (1-g) in the mixture may be from 5% to 100%, such as 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40% 10% to 35%, 10% to 30% 10% to 25%, 10% to 20%, 10% to 15%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, 50% to 55%, 60% to 90%, 60% to 85%, 60% to 80%, 60% to 75%, 60% to 70%, 60% to 65%, 70% to 90%, 70% to 85%, 70% to 80%, 70% to 75%, 80% to 90%, or 80% to 85% and the remainder percentage is compound (1-h). In some embodiments, the particular weight percentages of compound (1-g) in the mixture may be 87%, 88%, 89%, 90%, 91%, 92%, 93%, 94%, 95%, 96%, 97%, 98%, 99%, 99.5%, or 99.9%, and the remainder percentage is compound (1-h). In some embodiments, the particular weight percentages of compound (1-g) in the mixture may be greater than 87%, 88%, 89%, 90%, 91%, 92%, 93%, 94%, 95%, 96%, 97%, 98%, 99%, 99.5%, or 99.9%, and the remainder percentage is compound (1-h).

In some embodiments, the particular weight percentages of compound (1-i) in the mixture may be from 5% to 100%, such as 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40% 10% to 35%, 10% to 30% 10% to 25%, 10% to 20%, 10% to 15%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, 50% to 55%, 60% to 90%, 60% to 85%, 60% to 80%, 60% to 75%, 60% to 70%, 60% to 65%, 70% to 90%, 70% to 85%, 70% to 80%, 70% to 75%, 80% to 90%, or 80% to 85%, and the remainder percentage is compound (1-j). In some embodiments, the particular weight percentages of compound (1-i) in the mixture may be 87%, 88%, 89%, 90%, 91%, 92%, 93%, 94%, 95%, 96%, 97%, 98%, 99%, 99.5%, or 99.9%, and the remainder percentage is compound (1-j). In some embodiments, the particular weight percentages of compound (1-i) in the mixture may be greater than 87%, 88%, 89%, 90%, 91%, 92%, 93%, 94%, 95%, 96%, 97%, 98%, 99%, 99.5%, or 99.9%, and the remainder percentage is compound (1-j).

In some embodiments, the particular weight percentages of compound (1-k) in the mixture may be from 5% to 100%, such as 5% to 90%, 5% to 85%, 5% to 80%, 5% to 75%, 5% to 70%, 5% to 65%, 5% to 60%, 5% to 55%, 5% to 50%, 5% to 45%, 5% to 40%, 5% to 35%, 5% to 30%, 5% to 25%, 5% to 20%, 5% to 15%, 5% to 10%, 10% to 90%, 10% to 85%, 10% to 80%, 10% to 75%, 10% to 70%, 10% to 65%, 10% to 60%, 10% to 55%, 10% to 50%, 10% to 45%, 10% to 40% 10% to 35%, 10% to 30% 10% to 25%, 10% to 20%, 10% to 15%, 15% to 90%, 15% to 85%, 15% to 80%, 15% to 75%, 15% to 70%, 15% to 65%, 15% to 60%, 15% to 55%, 15% to 50%, 15% to 45%, 15% to 40%, 15% to 35%, 15% to 30%, 15% to 25%, 15% to 20%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 40% to 90%, 40% to 85%, 40% to 80%, 40% to 75%, 40% to 70%, 40% to 65%, 40% to 60%, 40% to 55%, 40% to 50%, 40% to 45%, 50% to 90%, 50% to 85%, 50% to 80%, 50% to 75%, 50% to 70%, 50% to 65%, 50% to 60%, 50% to 55%, 60% to 90%, 60% to 85%, 60% to 80%, 60% to 75%, 60% to 70%, 60% to 65%, 70% to 90%, 70% to 85%, 70% to 80%, 70% to 75%, 80% to 90%, or 80% to 85%, and the remainder percentage is compound (1-1). In some embodiments, the particular weight percentages of compound (1-k) in the mixture may be 87%, 88%, 89%, 90%, 91%, 92%, 93%, 94%, 95%, 96%, 97%, 98%, 99%, 99.5%, or 99.9%, and the remainder percentage is compound (1-1). In some embodiments, the particular weight percentages of compound (1-k) in the mixture may be greater than 87%, 88%, 89%, 90%, 91%, 92%, 93%, 94%, 95%, 96%, 97%, 98%, 99%, 99.5%, or 99.9%, and the remainder percentage is compound (1-1).

### Methods of Treatment

In some embodiments, provided herein is a method of treating and/or preventing cancer in a subject in need thereof.

In some embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer. In some embodiments, the cancer is breast cancer, lung cancer, prostate cancer, pancreatic cancer, or ovarian cancer. In some embodiments, the cancer is breast cancer, lung cancer, or prostate cancer.

In some embodiments, the cancer is breast cancer. In embodiments, the breast cancer is metastatic or locally advanced. In some embodiments, the breast cancer is estrogen receptor positive (ER+) breast cancer (e.g., human epidermal growth factor receptor 2 negative (HER2−)). In some embodiments, the subject is human (e.g., patient).

In some embodiments, the method comprises administering to the subject (e.g. patient) a therapeutically effective amount of a compound disclosed herein (e.g., a compound of Formula 1 or a pharmaceutically acceptable salt thereof).

In some embodiments, the present disclosure is directed to a method of treating cancer (e.g., breast cancer) in a patient in need thereof, comprising administering to the patient a therapeutically effective amount of a compound, wherein the compound is represented by Formula 1 or is a pharmaceutically acceptable salt thereof:

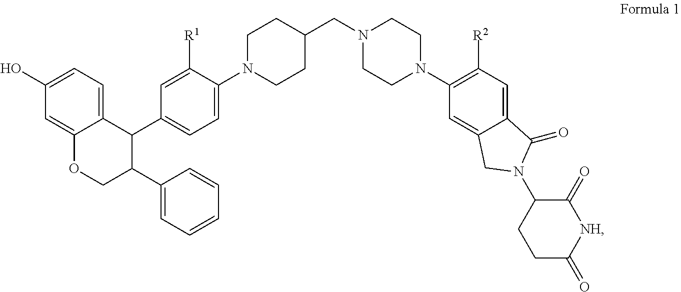

wherein:
R¹ is selected from H, Cl, Br, and F, and
R² is H or F, and
   wherein the therapeutically effective amount of the compound of Formula 1 is 20 mg to 1200 mg.

In some embodiments, R¹ and R² are both H.

In some embodiments, R¹ and R² are both F.

In some embodiments, R¹ is H and R² is F.

In some embodiments, wherein R¹ is F and R² is H.

In some embodiments, the compound of Formula 1 is selected from:

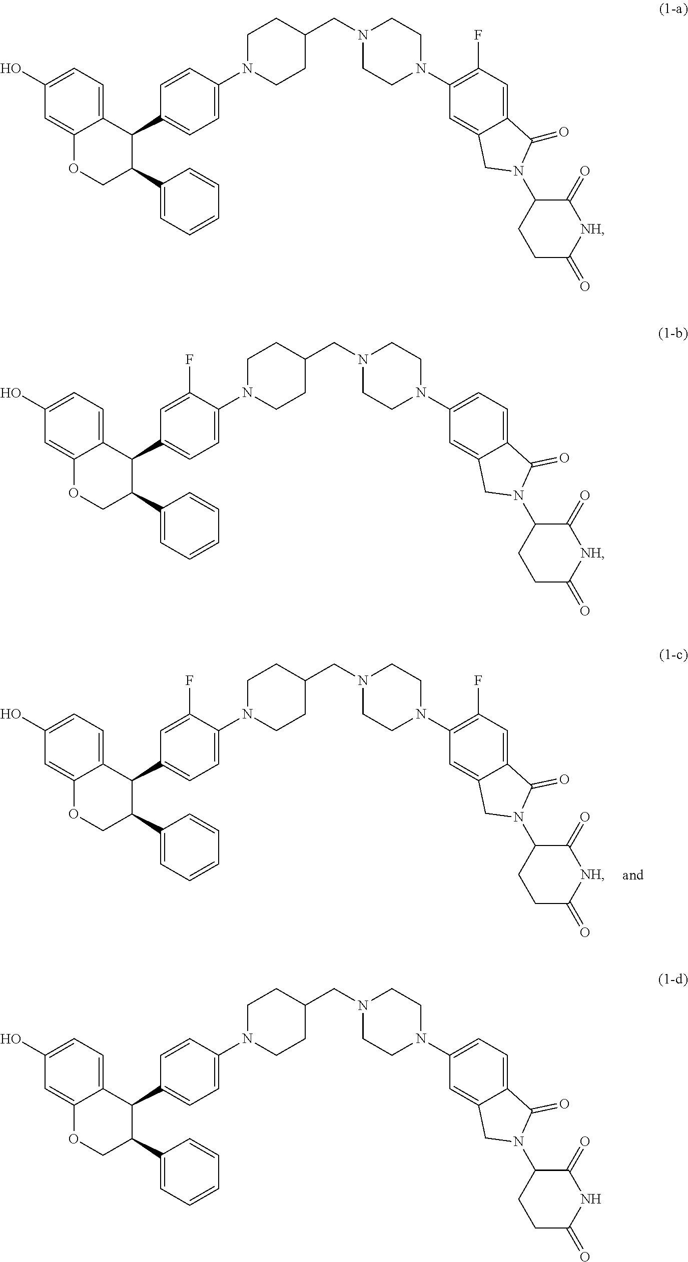 [SMILES: O=C1CCC(N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; "and" ; O=C1CCC(N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

or is a combination thereof.

In some embodiments, the compound of Formula 1 is

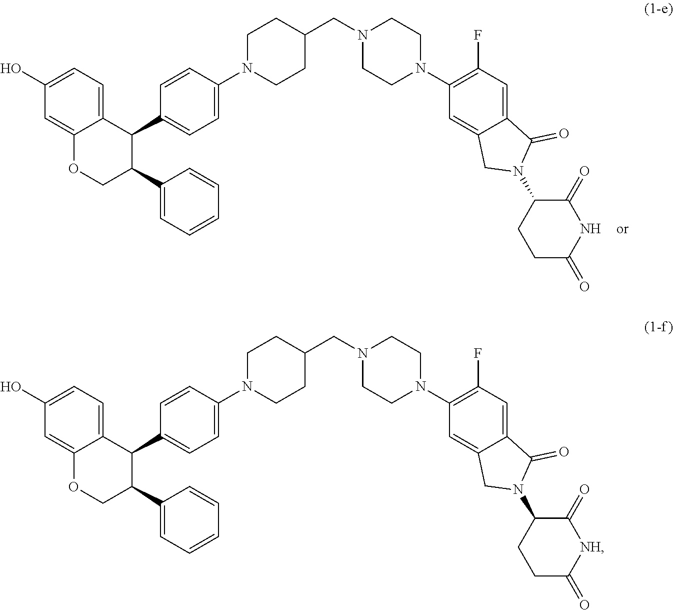 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1]

or is a combination thereof.

In some embodiments, the compound of Formula 1 is

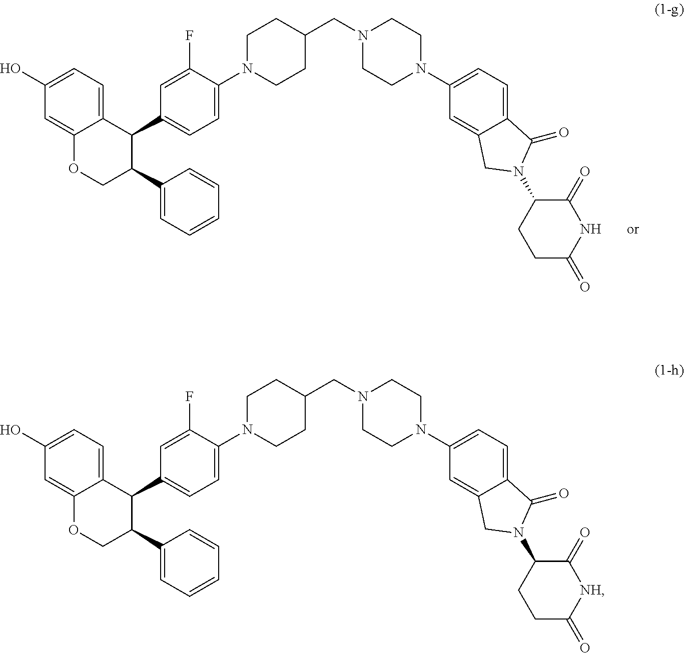 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

or is a combination thereof.

In some embodiments, the compound of Formula 1 is

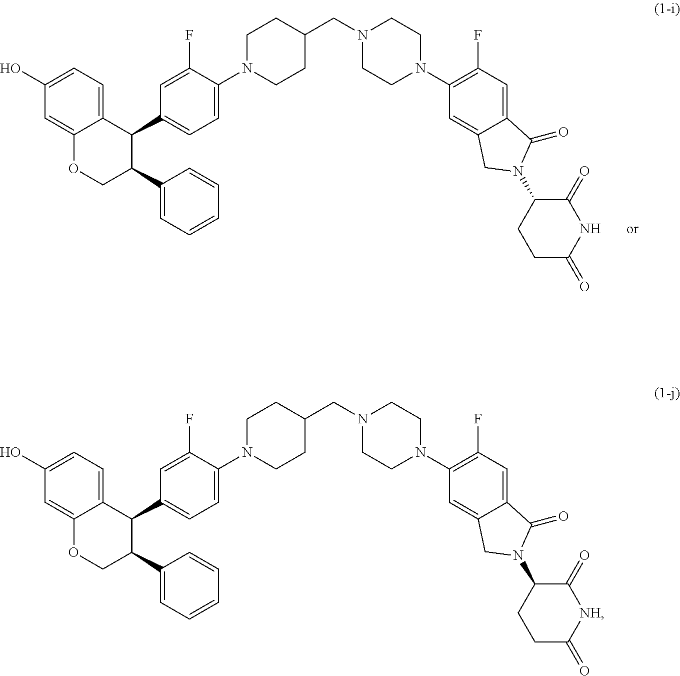 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1]

or is a combination thereof.

In some embodiments, the compound of Formula 1 is

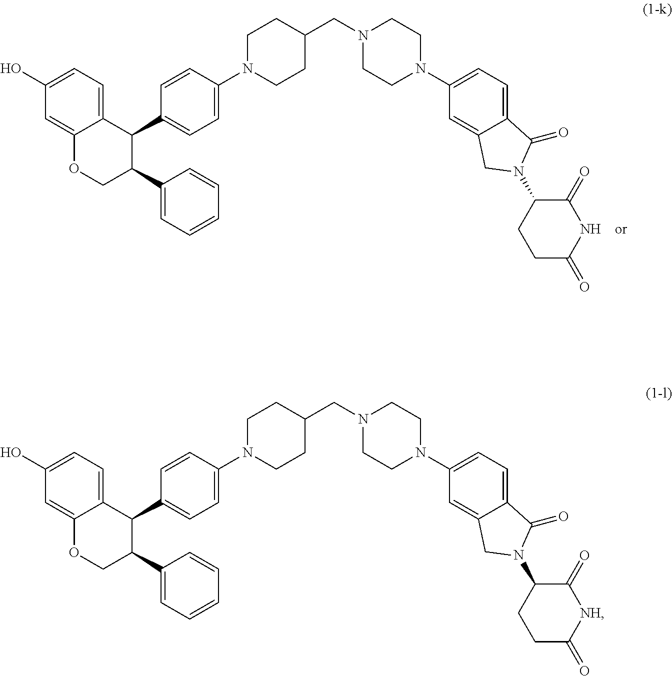 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

or is a combination thereof.

In some embodiments, the compound of Formula 1 is

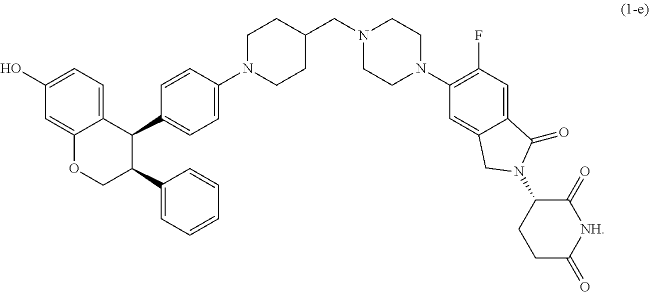 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1]

In some embodiments, the compound of Formula 1 is

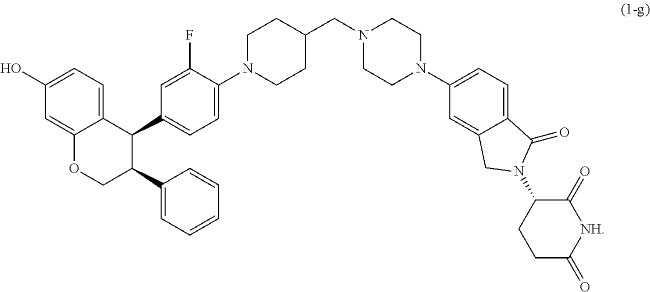 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

In some embodiments, the compound of Formula 1 is

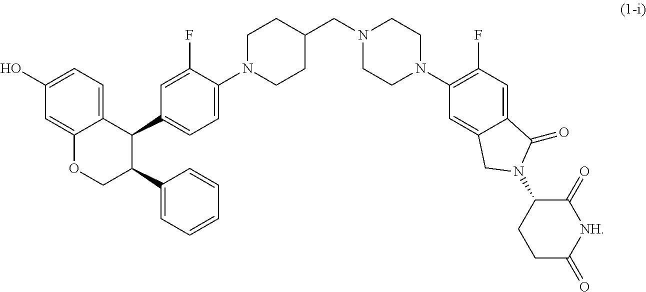 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1]

In some embodiments, the compound of Formula 1 is

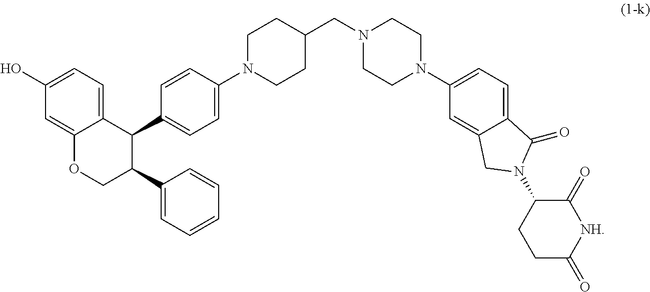 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

In some embodiments, the present disclosure is directed to a method of treating cancer, such as breast cancer, in a patient in need thereof, comprising administering to the patient a therapeutically effective amount of a compound, wherein the compound is represented by Formula 1 or is a pharmaceutically acceptable salt thereof:

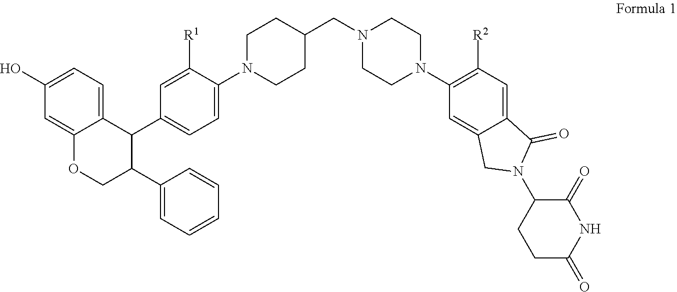

wherein:
R¹ is selected from H, Cl, Br, and F, and
R² is H or F, and
   wherein the therapeutically effective amount of the compound of Formula 1 is 20 mg to 1200 mg, 20 mg to 1000 mg, or 20 mg to 800 mg.

In some embodiments, the compound is selected from

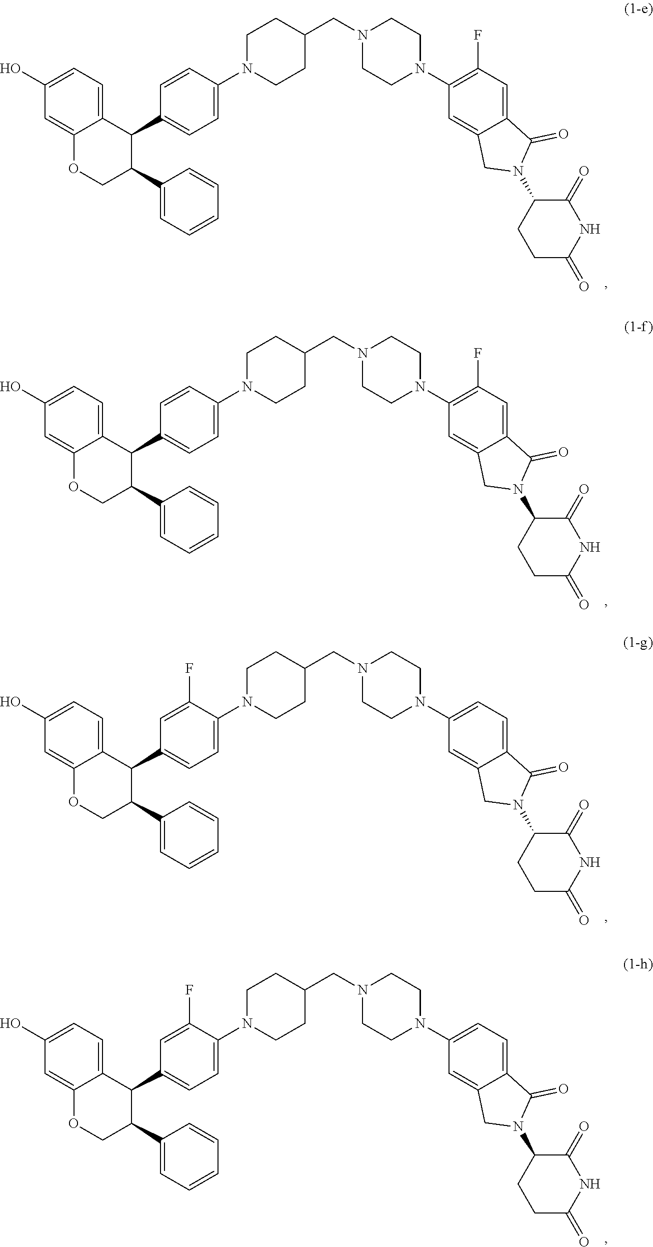 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

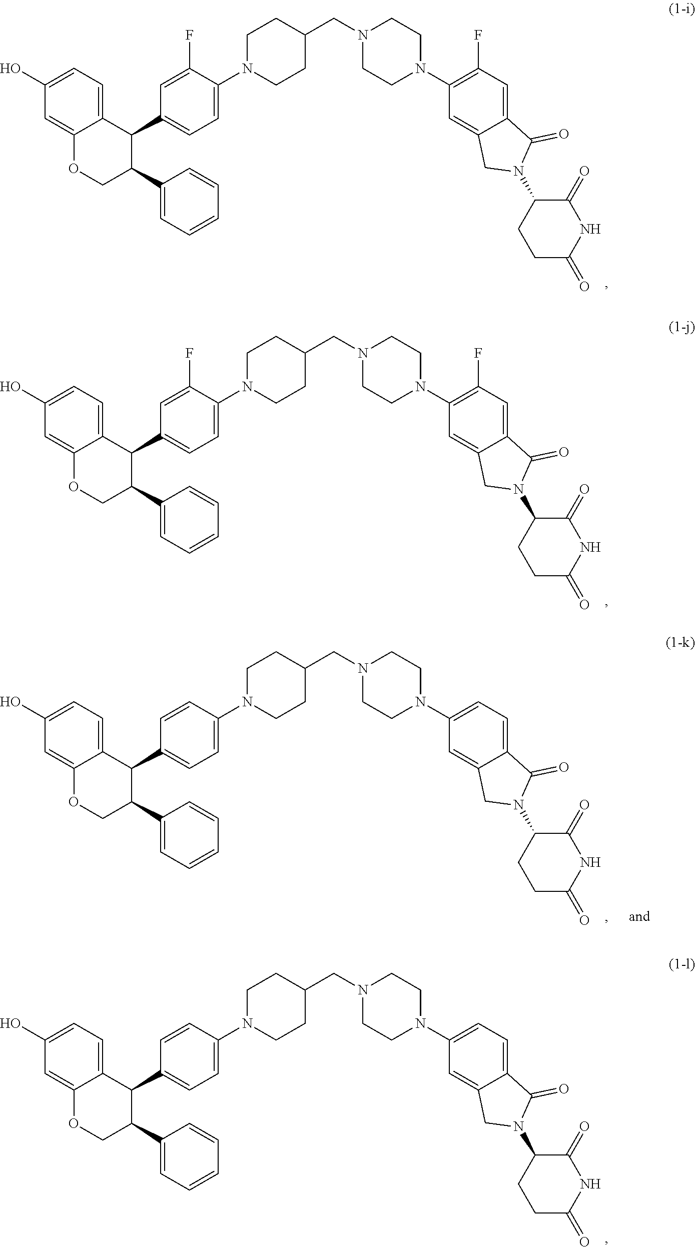 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

or is a combination thereof.

In some embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In some embodiments, the cancer is breast cancer, lung cancer, prostate cancer, pancreatic cancer, or ovarian cancer.

In some embodiments, the cancer is breast cancer, lung cancer, or prostate cancer.

In some embodiments, the cancer is breast cancer.

In some embodiments, the breast cancer is metastatic or locally advanced.

In some embodiments, the breast cancer is estrogen receptor positive (ER+) breast cancer.

In some embodiments, the estrogen receptor positive (ER+) breast cancer is human epidermal growth factor receptor 2 negative (HER2−).

In some embodiments, a compound of the present disclosure is selected from Table 1 or a pharmaceutically acceptable salt thereof.

TABLE 1
Compounds of the present disclosure

| Cpd # | Structure and IUPAC Name |
|---|---|
| 1-a | 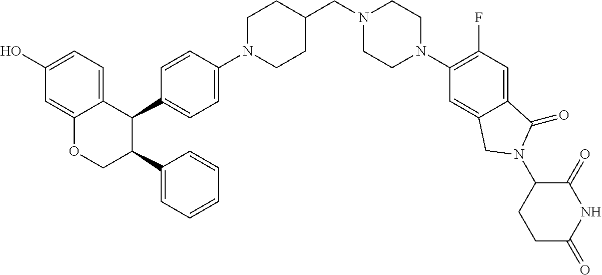 [SMILES: O=C1CCC(N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1] 3-(6-fluoro-5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-b | 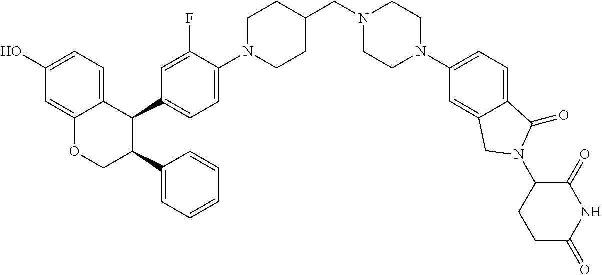 [SMILES: O=C1CCC(N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] 3-(5-(4-((1-(2-fluoro-4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-c | 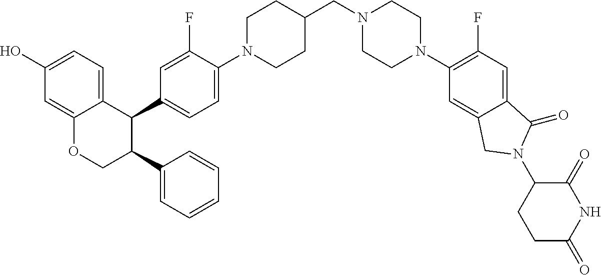 [SMILES: O=C1CCC(N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1] 3-(6-fluoro-5-(4-((1-(2-fluoro-4-((3S,4R)-7-hydroxy-3-phenylchroman-4- yl)phenyl)piperidin-4-yl)methyl)piperazin-1-yl)-1-oxoisoindolin-2-yl)piperidine-2,6-dione |
| 1-d | 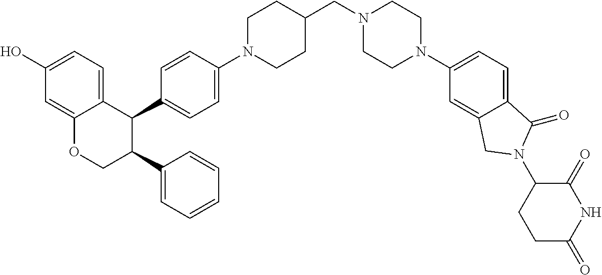 [SMILES: O=C1CCC(N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1] 3-(5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-e | 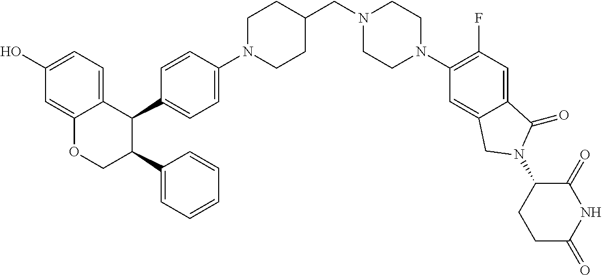 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1] (S)-3-(6-fluoro-5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-f | 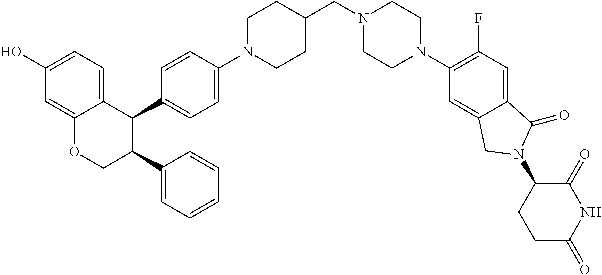 [SMILES: O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1] (R)-3-(6-fluoro-5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-g | 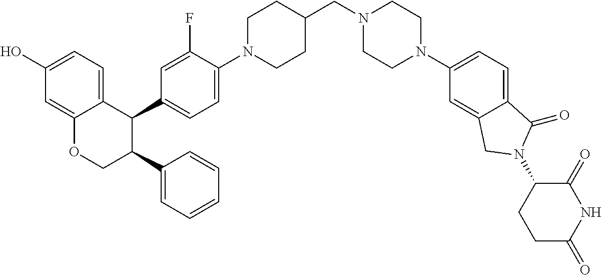 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] (S)-3-(5-(4-((1-(2-fluoro-4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-h | 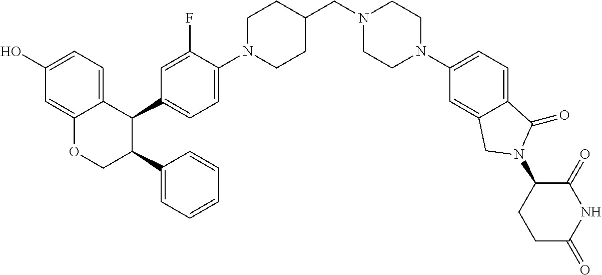 [SMILES: O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] (R)-3-(5-(4-((1-(2-fluoro-4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-i | 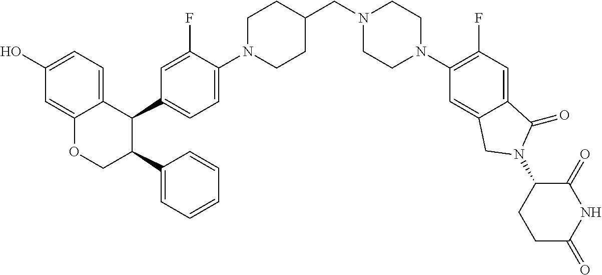 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1] (S)-3-(6-fluoro-5-(4-((1-(2-fluoro-4-((3S,4R)-7-hydroxy-3-phenylchroman-4- yl)phenyl)piperidin-4-yl)methyl)piperazin-1-yl)-1-oxoisoindolin-2-yl)piperidine-2,6-dione |
| 1-j | 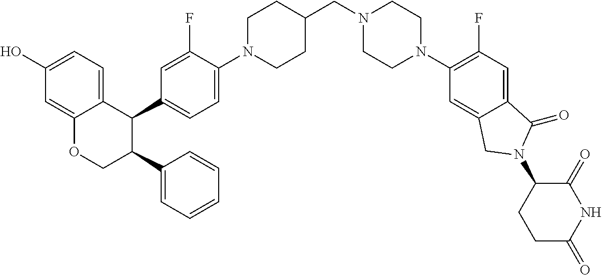 [SMILES: O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1] (R)-3-(6-fluoro-5-(4-((1-(2-fluoro-4-((3S,4R)-7-hydroxy-3-phenylchroman-4- yl)phenyl)piperidin-4-yl)methyl)piperazin-1-yl)-1-oxoisoindolin-2-yl)piperidine-2,6-dione |
| 1-k | 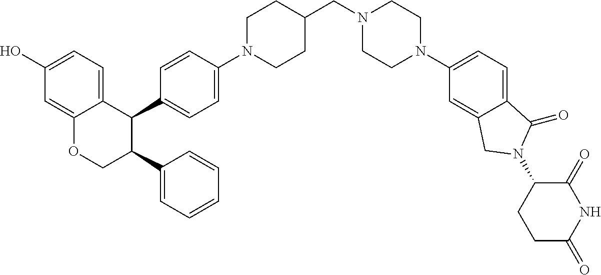 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1] (S)-3-(5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |
| 1-l | 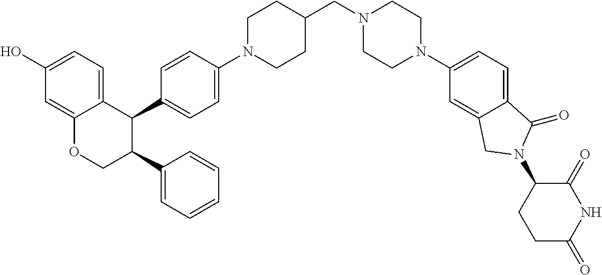 [SMILES: O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1] (R)-3-(5-(4-((1-(4-((3S,4R)-7-hydroxy-3-phenylchroman-4-yl)phenyl)piperidin-4- yl)methyl)piperazin-1-yl)-1-oxoisoindolin-2-yl)piperidine-2,6-dione |

In some embodiments, the disclosure provides for methods of treating cancer (e.g., breast cancer) in a subject in need thereof, the method comprising administering to the subject a therapeutically effective amount of a compound of Formula 1 or a pharmaceutically acceptable salt thereof:

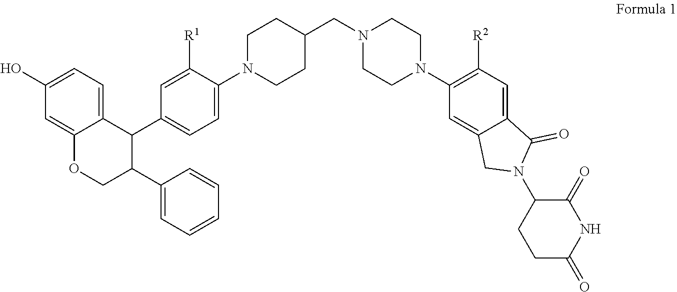

wherein:
R¹ is selected from H, Cl, Br, and F, and
R² is H or F, and
   wherein the therapeutically effective amount of the compound of Formula 1 is 20 mg to 1200 mg.

In some embodiments, the breast cancer is estrogen receptor positive/human epidermal growth factor receptor 2-negative (ER+/HER2−).

In some embodiments, the breast cancer is metastatic or locally advanced.

In some embodiments, the breast cancer is metastatic. In some embodiments, the breast cancer is locally advanced.

In some embodiments, the breast cancer comprises at least one Estrogen Receptor 1 (ESR1) tumor mutation.

In some embodiments, the at least one ESR1 tumor mutation is selected from Y537Z, D538Z, E380Z, L379Z, S463Z, and L536Z, wherein “Z” is an amino acid residue other than the wild-type residue at that position.

In some embodiments, the at least one ESR1 tumor mutation is selected from Y537S, Y537N, Y537C, D538G, E380Q, L3791, S463P, L536P, L536H, and L536R.

In some embodiments, the therapeutically effective amount of the compound disclosed herein is administered to the subject (e.g., patient) once a day, twice a day, three times a day, or four times a day. In some embodiments, the therapeutically effective amount of the compound is administered to the patient once a day. In some embodiments, the therapeutically effective amount of the compound disclosed herein is administered to the subject all at once or is administered in two, three, or four unit doses.

In some embodiments, following administration of the compound of Formula 1, the patient does not exhibit a dose limiting toxicity (DLT), a dose reduction, or one or more treatment-related adverse effects. In some embodiments, the patient, prior to administering the compound of Formula 1, had undergone at least one other cancer therapy (e.g., systemic therapy, endocrine therapy). In some embodiments, the at least one other cancer therapy comprises treatment with a CDK4/6 inhibitor, aromatase inhibitor, selective estrogen receptor degrader (SERD), covalent antagonist (SERCA), or an ER chimeric degrader. In some embodiments, the at least one other cancer therapy is administering a SERD, e.g., wherein the SERD is fulvestrant.

In some embodiments, at least one additional anti-cancer agent is administered to a patient in need thereof. In some embodiments, the at least one additional anti-cancer agent is selected from a FLT-3 inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, a CDK inhibitor (e.g., a CDK4 inhibitor, or a CDK4/6 inhibitor), anti-HGF antibody, PI3K kinase inhibitor, AKT inhibitor, mTORC1/2 inhibitor, JAK/STAT inhibitor, PD-1 inhibitor, PD-L1 inhibitor, B7-H3 inhibitor, CTLA4 inhibitor, LAG-3 inhibitor, OX40 agonist, an antibody (e.g., a VEGF trap antibody), and an antibody drug conjugate (ADC)), particularly a CDK4/6 inhibitor.

In some embodiments, the additional anti-cancer agent is a CDK4/6 inhibitor (e.g., palbociclib, ribociclib, abemaciclib). In some embodiments, the additional anti-cancer agent is a CDK4 inhibitor (e.g., atirmociclib (PF-07220060)). In some embodiments, the additional anti-cancer agent is a mTORC1/2 inhibitor (e.g., everolimus (Afinitor®)). In some embodiments, the additional anti-cancer agent is an AKT inhibitor (e.g., capivasertib (Truqap™)). In some embodiments, the additional anti-cancer agent is a PI3K inhibitor (e.g., copanlisib, alpelisib (Vijoice®), idelalisib, duvelisib, umbralisib). In some embodiments, the ADC is sacituzumab govitecan-hziy (Trodelvy®) or fam-trastuzumab deruxtecan-nxki (Enhertu®).

In some embodiments, the additional anti-cancer agent is selected from SHR6390, trilaciclib, lerociclib, AT7519M, dinaciclib, ribociclib, abemaciclib, atirmociclib (PF-07220060), palbociclib, everolimus (Afinitor®), venetoclax, inavolisib, pazopanib, carboplatin, capivasertib (Truqap™), cisplatin, oxaliplatin, paclitaxel, epithilone B, fulvestrant, acolbifene, lasofoxifene, idoxifene, topotecan, pemetrexed, erlotinib, ticilimumab, ipilimumab, vorinostat, etoposide, gemcitabine, doxorubicin, 5′-deoxy-5-fluorouridine, vincristine, temozolomide, capecitabine, camptothecin, PD0325901, irinotecan, tamoxifen, toremifene, anastrazole, letrozole, bevacizumab, goserelin acetate, raloxifene, alpelisib (Vijoice®), sacituzumab govitecan-hziy (Trodelvy®), trastuzumab, trastuzumab emtansine, pertuzumab, fam-trastuzumab deruxtecan-nxki (Enhertu®), and eribulin (halaven).

In some embodiments, the present disclosure is directed to a method of treating breast cancer in a patient in need thereof, wherein the breast cancer comprises at least one ESR1 tumor mutation or ESR1-wild-type.

In some embodiments, the method comprises orally administering to the patient once daily or twice daily a therapeutically effective amount of a compound, wherein the compound is selected from the compounds disclosed herein. In some embodiments, the compound is orally administering to the patient once daily. In some embodiments, the compound is orally administering to the patient twice daily.

In some embodiments the compounds are selected from:

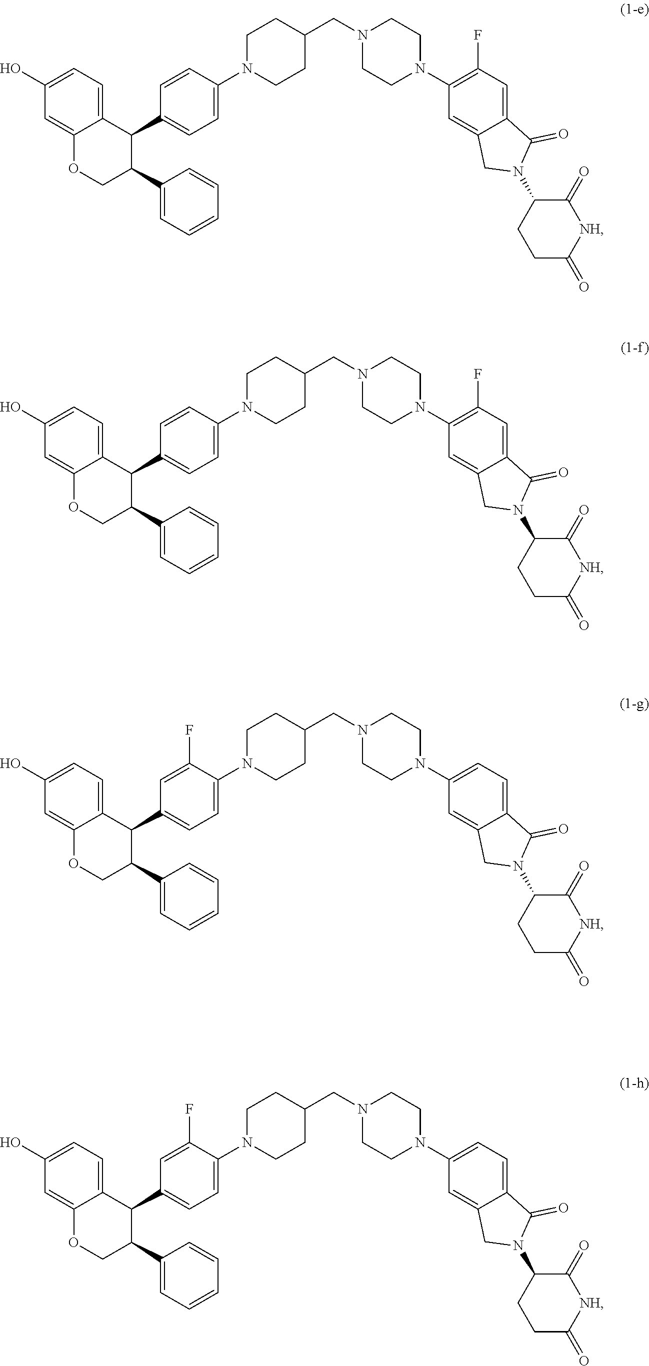 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

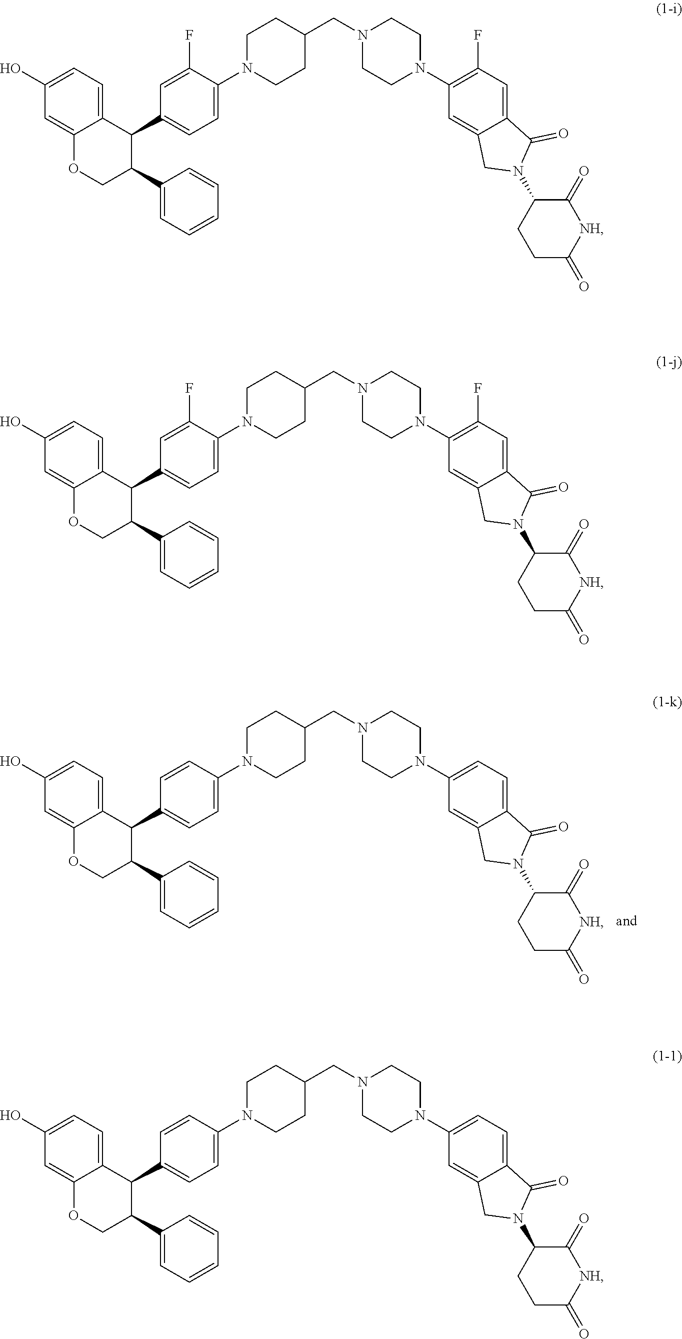 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

or pharmaceutically acceptable salts thereof.

In some embodiments, the compound is

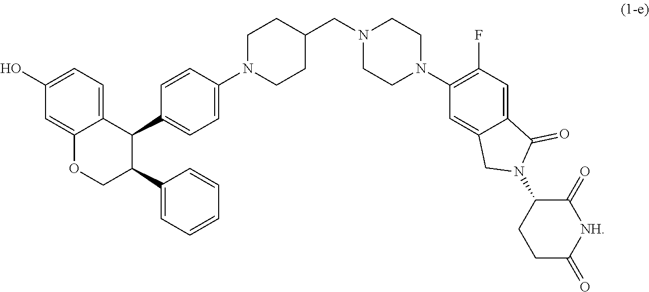 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1]

In some embodiments, the compound is

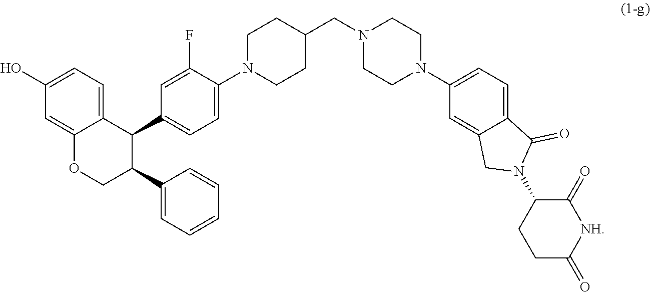 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

In some embodiments, the compound is

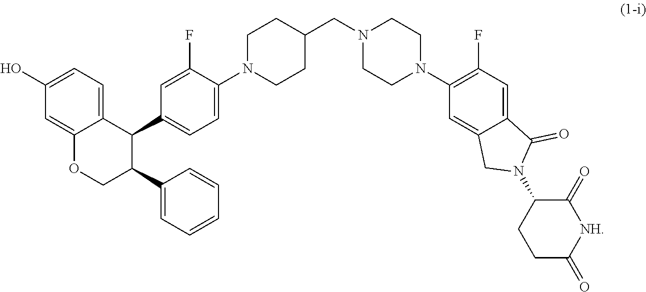 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1]

In some embodiments, the compound is

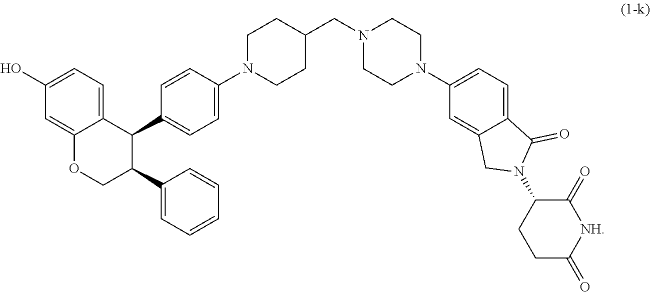 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

In some embodiments, the at least one ESR1 tumor mutation is selected from Y537Z, D538Z, E380Z, L379Z, S463Z, and L536Z, wherein “Z” is an amino acid residue other than the wild-type residue at that position.

In some embodiments, the at least one ESR1 tumor mutation is selected from Y537S, Y537N, Y537C, D538G, E380Q, L379I, S463P, L536P, L536H, and L536R.

In some embodiments, the breast cancer is estrogen receptor positive/human epidermal growth factor receptor 2-negative (ER+/HER2−).

In some embodiments, the breast cancer is metastatic or locally advanced.

In some embodiments, the therapeutically effective amount of the compound of Formula 1 is 20 mg to 1200 mg. In some embodiments, the therapeutically effective amount of the compound of Formula 1 is 100 mg. In some embodiments, the therapeutically effective amount of the compound of Formula 1 is 200 mg. In some embodiments, the therapeutically effective amount of the compound of Formula 1 is 300 mg. In some embodiments, the therapeutically effective amount of the compound of Formula 1 is 400 mg. In some embodiments, the therapeutically effective amount of the compound of Formula 1 is 500 mg. In some embodiments, the therapeutically effective amount of the compound of Formula 1 is 600 mg.

In some embodiments, the patient is administered the compound of Formula 1 for at least one month. In some embodiments, the patient is administered the compound of Formula 1 for at least three months. In some embodiments, the patient is administered the compound of Formula 1 for at least six months.

In some embodiments, following administration of the compound of Formula 1, the patient achieves at least a stable disease (SD). In some embodiments, following administration of the compound of Formula 1, the patient achieves at least a partial response (PR). In some embodiments, following administration of the compound of Formula 1, the patient achieves a complete response (CR).

In some embodiments, the patient, prior to administering the compound of Formula 1, has undergone at least one other cancer therapy (e.g., systemic therapy, endocrine therapy). In some embodiments, the at least one other cancer therapy comprises treatment with a CDK4/6 inhibitor, an aromatase inhibitor, a selective estrogen receptor degrader (SERD), a covalent antagonist (SERCA), or an ER chimeric degrader. In some embodiments, SERD includes a selective estrogen receptor covalent antagonist.

In some embodiments, the therapeutically effective amount of a compound disclosed herein (e.g., compound of Formula 1) results in AUC of greater than 11,000 ng*hr/mL. In some embodiments, the therapeutically effective amount of a compound disclosed herein results in AUC of greater than 12,000 ng*hr/mL. In some embodiments, the therapeutically effective amount of a compound disclosed herein results in AUC of greater than 13,000 ng*hr/mL. In some embodiments, the therapeutically effective amount of a compound disclosed herein results in AUC of greater than 14,000 ng*hr/mL. In some embodiments, the therapeutically effective amount of a compound disclosed herein results in AUC of greater than 15,000 ng*hr/mL. In some embodiments, the therapeutically effective amount of a compound disclosed herein results in AUC of greater than 16,000 ng*hr/mL. In some embodiments, the therapeutically effective amount of a compound disclosed herein results in AUC of greater than 17,000 ng*hr/mL. In some embodiments, the therapeutically effective amount of a compound disclosed herein results in AUC of greater than 18,000 ng*hr/mL. In some embodiments, the therapeutically effective amount of a compound disclosed herein results in AUC of greater than 18,500 ng*hr/mL. In some embodiments, the therapeutically effective amount of a compound disclosed herein results in AUC of greater than 19,000 ng*hr/mL. In some embodiments, the therapeutically effective amount of a compound disclosed herein results in AUC of greater than 19,500 ng*hr/mL. In some embodiments, the therapeutically effective amount of a compound disclosed herein results in AUC of greater than 20,000 ng*hr/mL. In some embodiments, the therapeutically effective amount of a compound disclosed herein results in AUC of greater than 20,500 ng*hr/mL. In some embodiments, the therapeutically effective amount of a compound disclosed herein results in AUC of greater than 21,000 ng*hr/mL. In some embodiments, the therapeutically effective amount of a compound disclosed herein results in AUC of greater than 21,500 ng*hr/mL. In some embodiments, the therapeutically effective amount of a compound disclosed herein results in AUC of greater than 21,800 ng*hr/mL. In some embodiments, the therapeutically effective amount of a compound disclosed herein results in AUC of greater than 25,000 ng*hr/mL. In some embodiments, the therapeutically effective amount of a compound disclosed herein results in AUC of greater than 30,000 ng*hr/mL. In some embodiments, the therapeutically effective amount of a compound disclosed herein results in AUC of greater than 35,000 ng*hr/mL. In some embodiments, the therapeutically effective amount of a compound disclosed herein results in AUC of greater than 38,500 ng*hr/mL.

In some embodiments, the therapeutically effective amount of a compound disclosed herein (e.g. a compound of Formula 1) results in a Cₘₐₓ of greater than 600 ng/ml, 630 ng/mL, 640 ng/ml, 700 ng/ml, 800 ng/mL, 900 ng/mL, 1000 ng/ml, 1010 ng/ml, 1020 ng/mL, 1030 ng/mL, 1040 ng/ml, 1050 ng/ml, 1060 ng/ml, 1070 ng/mL, 1080 ng/ml, 1100 ng/ml, 1100 ng/ml, 1200 ng/mL, 1300 ng/mL, 1400 ng/mL, 1500 ng/ml, 1600 ng/mL, 1700 ng/ml, 1800 ng/ml, 1900 ng/ml, 2000 ng/ml, 2100 ng/mL. In some embodiments, the therapeutically effective amount of a compound disclosed herein results in a Cₘₐₓ of greater than 2000 ng/ml, 2010 ng/ml, 2020 ng/mL, 2030 ng/ml, 2040 ng/mL, 2050 ng/ml, 2060 ng/ml, 2070 ng/ml, 2080 ng/mL, 2090 ng/ml, or 2090 ng/mL.

In some embodiments, the compound is administered orally to the patient. In some embodiments, the compound is administered to the patient once or twice a day. In some embodiments, the compound is administered to the patient once a day. In some embodiments, the compound is administered to the patient twice a day. In some embodiments, the therapeutically effective amount of the compound is 50 mg to 1000 mg. In some embodiments, the therapeutically effective amount of the compound is 100 mg to 600 mg.

In some embodiments, the therapeutically effective amount of the compound disclosed herein (e.g., Formula 1) is 50 to 70 mg, 60 to 80 mg, 70 to 90 mg, 80 to 100 mg, 90 to 120 mg, 100 to 130 mg, 110 to 140 mg, 120 to 150 mg, 130 to 160 mg, 140 to 170 mg, 150 to 180 mg, 160 to 190 mg, 170 to 200 mg, 180 to 210 mg, 190 to 220 mg, 200 to 230 mg, 210 to 240 mg, 220 to 250 mg, 230 to 260 mg, 240 to 270 mg, 250 to 280 mg, 260 to 290 mg, 270 to 300 mg, 280 to 310 mg, 290 to 320 mg, 300 to 330 mg, 310 to 340 mg, 320 to 350 mg, 330 to 360 mg, 340 to 370 mg, 350 to 380 mg, 360 to 390 mg, 370 to 400 mg, 400 to 430 mg, 410 to 440 mg, 420 to 450 mg, 430 to 460 mg, 440 to 470 mg, 450 to 480 mg, 460 to 490 mg, 470 to 500 mg, 500 to 530 mg, 510 to 540 mg, 520 to 550 mg, 530 to 560 mg, 540 to 570 mg, 550 to 580 mg, 560 to 590 mg, or 570 to 600 mg. In some embodiments, the therapeutically effective amount of the compound is 100 mg. In some embodiments, the therapeutically effective amount of the compound is 200 mg. In some embodiments, the therapeutically effective amount of the compound is 250 mg. In some embodiments, the therapeutically effective amount of the compound is 300 mg. In some embodiments, the therapeutically effective amount of the compound is 350 mg. In some embodiments, the therapeutically effective amount of the compound is 400 mg. In some embodiments, the therapeutically effective amount of the compound is 450 mg. In some embodiments, the therapeutically effective amount of the compound is 500 mg. In some embodiments, the therapeutically effective amount of the compound is 550 mg. In some embodiments, the therapeutically effective amount of the compound is 600 mg.

In some embodiments, the cancer is breast cancer. In some embodiments, the compounds used in the methods disclosed herein induce more potent and deeper degradation of ER. The compounds disclosed herein are composed of a linker connected to a ligand that binds a protein of interest with a ligand that binds E3 ligase. This subsequently brings the protein of interest in proximity to the E3 ligase, resulting in robust ubiquitination of the protein of interest and its subsequent degradation. In some embodiments, the compounds disclosed herein may degrade the ER protein without the risk of signal activation. In some embodiments, the compounds disclosed herein are not degraded along with the target protein and as such can be recycled within a cell. This allows the compounds disclosed herein to achieve potent ER degradation and treatment efficacy at a lower drug concentration with lower associated toxicities.

In some embodiments, the methods of treating cancer disclosed herein include a reduction in tumor size. In some embodiments, the cancer is metastatic cancer, and the method of treatment includes inhibition of metastatic cancer cell invasion. In some embodiments, the cancer is breast cancer. In some embodiments, the breast cancer is metastatic breast cancer. In some embodiments, the breast cancer is locally advanced breast cancer. In some embodiments, the breast cancer is ER+, HER2− breast cancer. In some embodiments, the breast cancer is metastatic, ER+, HER2− breast cancer. In some embodiments, the breast cancer is metastatic, ER+, HER2− breast cancer that is also locally advanced.

In some embodiments, treating cancer results in a reduction in size of a tumor lesion. A reduction in size of a tumor may also be referred to as “tumor regression.” A reduction in size of a tumor may also be referred to as “lesion reduction.” In some embodiments, after treatment, tumor lesion size is reduced by 5% or greater relative to its size prior to treatment. In some embodiments, tumor lesion size is reduced by 10% or greater. In some embodiments, tumor lesion size is reduced by 20% or greater. In some embodiments, tumor lesion size is reduced by 30% or greater. In some embodiments, tumor lesion size is reduced by 40% or greater. In some embodiments, tumor lesion size is reduced by 50% or greater. In some embodiments, tumor lesion size is reduced by greater than 75% or greater. In some embodiments, tumor lesion size of a tumor lesion may be measured by any reproducible means of measurement. In some embodiments, tumor lesion size may be measured as a diameter of the tumor.

In some embodiments, treating cancer results in a reduction in tumor lesion diameter. In some embodiments, after treatment, tumor lesion diameter is reduced by 5% or greater relative to its size prior to treatment. In some embodiments, after treatment, tumor lesion diameter is reduced by 10% or greater. In some embodiments, after treatment, tumor lesion diameter is reduced by 20% or greater. In some embodiments, after treatment, tumor lesion diameter is reduced by 30% or greater. In some embodiments, after treatment, tumor diameter is reduced by 40% or greater. In some embodiments, after treatment, tumor lesion diameter is reduced by 50% or greater. In some embodiments, after treatment, tumor lesion is reduced by greater than 75% or greater. Tumor lesion may be measured by any reproducible means of measurement.

In some embodiments, treating cancer results in a decrease in number of tumor lesions. In some embodiments, after treatment, tumor lesion number is reduced by 5% or greater relative to number prior to treatment. In some embodiments, after treatment, tumor lesion number is reduced by 10% or greater. In some embodiments, after treatment, tumor lesion number is reduced by 20% or greater. In some embodiments, after treatment, tumor lesion number is reduced by 30% or greater. In some embodiments, after treatment, tumor lesion number is reduced by 40% or greater. In some embodiments, after treatment, tumor lesion number is reduced by 50% or greater. In some embodiments, after treatment, tumor lesion number is reduced by greater than 75%. Number of tumor lesions may be measured by any reproducible means of measurement. In some embodiments, number of tumor lesions may be measured by counting tumors visible to the naked eye or at a specified magnification.

In some embodiments, treating cancer results in a decrease in number of metastatic lesions in other tissues or organs distant from the primary tumor site. In some embodiments, after treatment, the number of metastatic lesions is reduced by 5% or greater relative to number prior to treatment. In some embodiments, the number of metastatic lesions is reduced by 10% or greater. In some embodiments, the number of metastatic lesions is reduced by 20% or greater. In some embodiments, the number of metastatic lesions is reduced by 30% or greater. In some embodiments, the number of metastatic lesions is reduced by 40% or greater. In some embodiments, the number of metastatic lesions is reduced by 50% or greater. In some embodiments, the number of metastatic lesions is reduced by 60% or greater. In some embodiments, the number of metastatic lesions is reduced by 70% or greater. In some embodiments, the number of metastatic lesions is reduced by greater than 75%. The number of metastatic lesions may be measured by any reproducible means of measurement. In some embodiments, the number of metastatic lesions may be measured by counting metastatic lesions visible to the naked eye or at a specified magnification.

In some embodiments, treating cancer results in an increase in average survival time of a population of treated subjects. In some embodiments, the average survival time is increased by more than 30 days. In some embodiments, the average survival time is increased by more than 60 days. In some embodiments, the average survival time is increased by more than 90 days. In some embodiments, the average survival time is increased by more than 120 days. An increase in average survival time of a population may be measured by any reproducible means.

In some embodiments, treating cancer results in an increase in average survival time of a population of treated subjects in comparison to a population of untreated subjects. In some embodiments, the average survival time is increased by more than 30 days. In some embodiments, the average survival time is increased by more than 60 days. In some embodiments, the average survival time is increased by more than 90 days. In some embodiments, the average survival time is increased by more than 120 days. An increase in average survival time of a population may be measured by any reproducible means.

In some embodiments, treating cancer results in a decrease in tumor growth rate. In some embodiments, after treatment, tumor growth rate is reduced by at least 5% relative to number prior to treatment. In some embodiments, tumor growth rate is reduced by at least 10%. In some embodiments, tumor growth rate is reduced by at least 20%. In some embodiments, tumor growth rate is reduced by at least 30%. In some embodiments, tumor growth rate is reduced by at least 40%. In some embodiments, tumor growth rate is reduced by at least 50%. In some embodiments, tumor growth rate is reduced by at least 50%. In some embodiments, tumor growth rate is reduced by at least 75%. Tumor growth rate may be measured by any reproducible means of measurement. In some embodiments, tumor growth rate is measured according to a change in tumor diameter per unit time.

In some embodiments, treating cancer results in a decrease in tumor regrowth. In some embodiments, after treatment, tumor regrowth is less than 5%. In some embodiments, tumor regrowth is less than 10%. In some embodiments, tumor regrowth is less than 20%. In some embodiments, tumor regrowth is less than 30%. In some embodiments, tumor regrowth is less than 40%. In some embodiments, tumor regrowth is less than 50%. In some embodiments, tumor regrowth is less than 50%. In some embodiments, tumor regrowth is less than 75%. Tumor regrowth may be measured by any reproducible means of measurement. In some embodiments, tumor regrowth is measured, for example, by measuring an increase in the diameter of a tumor after a prior tumor shrinkage that followed treatment. In some embodiments, a decrease in tumor regrowth is indicated by failure of tumors to reoccur after treatment has stopped. In some embodiments, a decrease in tumor is measured as a change in sum of target lesions from baseline. In some embodiments, the decrease in tumor growth is in patients with ESR1 mutation. In some embodiments, the decrease in tumor growth is in patients with wild type (WT) ER.

The dosages of a compound disclosed herein for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of a compound disclosed herein (e.g., Formula 1) may be administered one, two, three, four, five, or more times over a day for 1, 2, 3, 4, 5, 6, 7, 8, 9, 10, 11, 12, 13, 14, 15, 16, 17, 18, 19, 20, 21, 22, 23, 24, 25, 26, 27, 28 or more days, followed by at least one or more days of non-administration of a compound of Formula 1. This type of treatment schedule, i.e., administration of a compound of Formula 1 or a pharmaceutically acceptable salt thereof on consecutive days followed by non-administration of the compound on consecutive days may be referred to as a treatment cycle.

In some embodiments, the therapeutically effective amount of the compound disclosed herein is administered to the subject once a day or twice a day. In some embodiments, the therapeutically effective amount of the compound is administered to the subject all at once or is administered in two, three, or four unit doses. In some embodiments, the therapeutically effective amount of the compound is 50 mg to 1000 mg. In some embodiments, the therapeutically effective amount of the compound is 50 mg to 1000 mg. In some embodiments, the therapeutically effective amount of the compound is 100 mg to 600 mg. In some embodiments, the therapeutically effective amount of the compound is 100 mg. In some embodiments, the therapeutically effective amount of the compound is 200 mg. In some embodiments, the therapeutically effective amount of the compound is 250 mg. In some embodiments, the therapeutically effective amount of the compound is 300 mg. In some embodiments, the therapeutically effective amount of the compound is 350 mg. In some embodiments, the therapeutically effective amount of the compound is 400 mg. In some embodiments, the therapeutically effective amount of the compound is 450 mg. In some embodiments, the therapeutically effective amount of the compound is 500 mg. In some embodiments, the therapeutically effective amount of the compound is 550 mg. In some embodiments, the therapeutically effective amount of the compound is 600 mg.

In some embodiments, a treatment cycle involving the compound of Formula 1 may be repeated as many times as necessary to achieve the intended affect. In some embodiments, the treatment schedule comprises one treatment cycle. In some embodiments, the treatment schedule comprises two treatment cycles. In some embodiments, the treatment schedule comprises three treatment cycles. In some embodiments, the treatment schedule comprises four treatment cycles.

In some embodiments, the present disclosure is directed to methods of treating breast cancer in a subject having a breast cancer comprising at least one somatic ER tumor mutation. In some embodiments, the present disclosure is directed to methods of treating breast cancer in a subject having a breast cancer comprising a wild-type ER.

In some embodiments, the compounds disclosed herein degrade the ER protein. In some embodiments, the ER that is degraded by the compounds disclosed herein (e.g. compounds of Formula 1) is wild type ER. In some embodiments, the ER that is degraded by the compounds disclosed herein is a mutant form of ER.

In some embodiments, the at least one somatic ER tumor mutation is selected from Y537X, D538X, E380X, L379X, V422X, S463X, and L536X, wherein “X” refers to any amino acid residue, other than the wild-type residue at that position. In some embodiments, the subject comprises at least one somatic ER tumor mutation selected from the group consisting of Y537S, Y537N, D538G, E380Q, L379I, V422del, S463P, L536P and L536_D538>P.

In some embodiments, the at least one somatic ER tumor mutation is selected from Y537X, D538X, E380X, L379X, V422X, S463X, and L536X, wherein “X” refers to any amino acid residue, other than the wild-type residue at that position, selected from alanine (A); valine (V); leucine (L); isoleucine (I); phenylalanine (F); methionine (M); tryptophan (W); proline (P); glycine (G); serine(S); threonine (T); cysteine (C); asparagine (N); glutamine (Q); tyrosine (Y); lysine (K); arginine (R); histidine (H); aspartate (D); and glutamate (E).

In some embodiments, the at least one somatic ER tumor mutation is Y537X.

In some embodiments, the at least one somatic ER tumor mutation is D538X.

In some embodiments, the at least one somatic ER tumor mutation is E380X.

In some embodiments, the at least one somatic ER tumor mutation is L379X.

In some embodiments, the at least one somatic ER tumor mutation is V422X.

In some embodiments, the at least one somatic ER tumor mutation is S463X.

In some embodiments, the at least one somatic ER tumor mutation is L536X.

In some embodiments, the breast cancer comprises cancer cells characterized by expression of at least one somatic ER tumor mutation described herein. Methods to identify a cancer characterized by expression of somatic mutations are known in the art, and include, e.g., obtaining a biological sample from the subject, harvesting the biological sample to obtain genetic material (e.g., genomic DNA or RNA), and performing sequencing analysis, RNA-sequencing analysis, or real-time polymerase chain reaction (RT-PCR). For example, in some embodiments, genomic DNA is first obtained (using any standard technique) from cancerous tissue obtained from the subject, cDNA is prepared, and amplification is performed (e.g., using a polymerase chain reaction) to provide the cDNA in sufficient quantity for sequence analysis, and sequencing is performed using, e.g., next generation sequencing. Genomic DNA or RNA is typically extracted from biological samples such as tissues removed from the subject, e.g., by tissue biopsy. In some embodiments, the biological sample is a tissue biopsy sample (e.g., a breast tumor biopsy sample), wherein sequence analysis of genomic DNA or RNA is performed to identify the presence of somatic mutations in the ER (e.g., a somatic ER tumor mutation present in the ER ligand binding domain). In some embodiments, the biological sample comprises plasma obtained from the subject is used to detect somatic ER tumor mutations present in circulating tumor DNA, e.g., using PCR based amplification and gene sequencing.

In some embodiments, the ER biomarker status of a subject suffering from breast cancer can be determined through an analysis of the subject's circulating tumor DNA (ctDNA) Alternative methods for determining the ER biomarker status of a subject suffering from breast cancer include, without limitation, fluorescent in situ hybridization, immunohistochemistry, PCR analysis, or sequencing.

In some embodiments, the ER biomarker status of a subject suffering from breast cancer is determined in a blood sample derived from the subject.

In some embodiments, the ER biomarker status of a subject suffering from breast cancer is determined in a solid biopsy derived from the tumor of the subject.

In some embodiments, the breast cancer patient is selected for treatment based on the presence of at least one somatic ER tumor mutation.

### Pharmaceutical Compositions

In some embodiments, the compound described herein is administered as a pure chemical. In other embodiments, the compound described herein is administered as a pharmaceutical composition when the compound is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration (e.g., oral administration) and standard pharmaceutical practice as described, for example, in Remington: The Science and Practice of Pharmacy (Gennaro, 21st Ed. Mack Pub. Co., Easton, PA (2005)).

In certain embodiments, the compound provided herein is substantially pure, in that it contains less than 25%, or less than 20%, or less than 15%, or less than 10%, or less than 5%, or less than 1%, or less than 0.1%, of other organic small molecules, such as unreacted intermediates, synthesis by-products that are created, for example, in one or more of the steps of a synthesis method, or degradation/interconversion products (e.g. epimers of the parent compound).

In certain embodiments, the pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

In some embodiments, clinical benefit rate (CBR) is evaluated. The clinical benefit rate (CBR), as used herein, is defined as confirmed complete response or partial response at any time and stable disease ≥24 weeks. In some embodiments, evaluable participants for the CBR evaluation need to complete an adequate post-treatment scan. In some embodiments, CBR includes patients with stable disease over ≥24 weeks. In some embodiments, evaluable patients harbor a confirmed ESR1 mutation. In some embodiments, CBR is from 10% to 100%, such as 15%, 20%, 25%, 30%, 35%, 40%, 45%, 50%, 55%, 60%, 65%, 70%, 75%, 80%, 85%, 90%, or 95%. In certain embodiments, CBR is from 20% to 65%, such as 25%, 30%, 35%, 40%, 45%, 50%, 55%, or 60%.

In some embodiments, objective response rate (ORR) is evaluated. In some embodiments, ORR is from 10% to 100%, such as 15%, 20%, 25%, 30%, 35%, 40%, 45%, 50%, 55%, 60%, 65%, 70%, 75%, 80%, 85%, 90%, or 95%. In certain embodiments, ORR is from 15% to 60%, such as 20%, 25%, 30%, 35%, 40%, 45%, 50%, or 55%.

In some embodiments, a disclosed compound or composition (e.g., an oral tablet composition) may be administered once daily to the patient. In some embodiments, a disclosed compound or composition (e.g., an oral tablet composition) may be administered twice daily to the patient. In some embodiments, for example, a disclosed compound or pharmaceutical composition is formulated for oral administration. In some embodiments, the pharmaceutical composition is formulated as a tablet, a pill, a capsule, a liquid, a suspension, a dispersion, a solution, or an emulsion. In some embodiments, the pharmaceutical composition is formulated as a tablet, for example, a tablet formulated for oral administration.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

In some embodiments, a pharmaceutical composition comprising at least one compound of Formula 1, including combinations or mixtures thereof, or a pharmaceutically acceptable salt thereof is provided. In some embodiments, the pharmaceutical composition comprises the compound of Formula 1, including combinations or mixtures thereof, or a pharmaceutically acceptable salt thereof, as the sole active ingredient(s). In other embodiments, the pharmaceutical composition further comprises an additional anti-cancer agent. In some embodiments, the pharmaceutical composition is formulated together with one or more pharmaceutically acceptable carriers. These formulations include those suitable for oral, rectal, topical, buccal, and parenteral (e.g., subcutaneous, intramuscular, intradermal, or intravenous) administration. The most suitable form of administration in any given case will depend on the degree and severity of the condition being treated and on the nature of the particular compound being used.

In some embodiments, oral compositions comprising at least one compound of Formula 1 or a pharmaceutically acceptable salt thereof are provided. Oral compositions generally include an inert diluent or an edible pharmaceutically acceptable carrier. They can be enclosed in gelatin capsules or compressed into tablets. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

In some embodiments, the pharmaceutical composition may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of a compound into preparations that can be used pharmaceutically. The appropriate formulation is dependent upon the route of administration chosen.

In some embodiments, the pharmaceutical composition can be administered according to the invention by any appropriate route, including oral, parenteral (subcutaneous, intramuscular, intravenous (bolus or infusion), depot, intraperitoneal), intrathecal, intranasal, intravaginal, sublingual, buccal, intraocular, or rectal.

In some embodiments, the pharmaceutical composition is formulated as a tablet comprising zero, one, two, or more of each of the following: emulsifier, surfactant, binder, disintegrant, glidant, and lubricant. In some embodiments, the pharmaceutical composition is formulated as a capsule. In some embodiments, the pharmaceutical composition is formulated as an oral liquid.

In some embodiments, the emulsifier is hypromellose. In some embodiments, the surfactant is vitamin E polyethylene glycol succinate. In some embodiments, the binder (also referred to herein as a filler) is selected from the group consisting of microcrystalline cellulose, lactose monohydrate, sucrose, glucose, and sorbitol.

In some embodiments, the disintegrant is croscarmellose sodium.

In some embodiments, the glidant refers to a substance used to promote powder flow by reducing interparticle cohesion. In some embodiments, in the dosage forms of the disclosure, the glidant is selected from the group consisting of silicon dioxide, silica colloidal anhydrous, starch, and talc.

In some embodiments, the lubricant refers to a substance that prevents ingredients from sticking and/or clumping together in the machines used in preparation of the dosage forms of the disclosure. In some embodiments, in the dosage forms of the disclosure, the lubricant is selected from the group consisting of magnesium stearate, sodium stearyl fumarate, stearic acid, and vegetable stearin.

In some embodiments, the pharmaceutical composition can be included in a container, pack, or dispenser together with instructions for administration. In some embodiments, the pharmaceutical composition can be packaged in a bottle. In some embodiments, the bottle is a polyethylene bottle. In some embodiments, the bottle is manufactured from high density polyethylene.

### Combination Therapy

In some embodiments, a compound or composition (e.g., a disclosed oral tablet composition) described herein is administered in combination with a second therapeutic agent, for example, at least one endocrine therapy including a CDK inhibitor, such as a CDK4/6 inhibitor.

In some embodiments, the CDK inhibitor may be administered at any suitable dose, such as 1 mg, 5 mg, 10 mg, 15 mg, 20 mg, 25 mg, 35 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, or 975 mg, including all values and ranges therebetween.

In some embodiments, the CDK inhibitor is palbociclib, ribociclib, or abemaciclib. The CDK inhibitor may be administered at the FDA approved dose or doses (i.e., as provided on the label approved by the FDA as of the filing date of this application), or at a reduced dose. As used herein, a “reduced dose” refers to a dose that is less than the approved dose as provided on the FDA approved label. In some embodiments, the reduced dose may be 10%, 15%, 20%, 25%, 30%, 35%, 40%, 45%, 50%, 55%, 60%, 65%, 70%, 75%, 80%, 85%, 90%, or 95% less than the approved dose.

In some embodiments, the method comprises administering from 25 mg to 200 mg palbociclib, from 50 mg to 150 mg palbociclib, from 50 mg to 100 mg palbociclib, from 75 mg to 125 mg palbociclib or from 100 mg to 150 mg palbociclib, including all ranges and values therebetween. In some embodiments, the oral dosage form comprises 125 mg, 100 mg, 75 mg, 50 mg, or 25 mg of palbociclib, including all values and ranges between these values.

In some embodiments, the method comprises administering ribociclib, such as ribociclib succinate, in amount ranging from 50 mg to 600 mg (e.g., 50, 100, 150, 200, 250, 300, 350, 400, 450, 500, 550, or 600 mg, including all values and ranges between these values, such as from 50 mg to 350 mg, from 100 mg to 300 mg, from 150 mg to 250 mg, or from 175 mg to 225 mg). In some embodiments, the oral dosage form comprises ribociclib succinate in 150 mg to 600 mg of the equivalent amount of ribociclib free base, including all ranges and values therebetween. In some embodiments, the oral dosage form comprises 200 mg of the equivalent amount of ribociclib free base.

In some embodiments, the methods disclosed herein comprises administering from 25 mg to 500 mg of abemaciclib (e.g., 25, 50, 75, 100, 125, 150, 175, 200, 225, 250, 275, 300, 325, 350, 375, 400, 425, 450, 475, 500, 525, 550, 575, or 600 mg, including all values and ranges between these values), from 25 mg to 250 mg, from 50 mg to 200 mg, from 25 mg to 75 mg, from 75 mg to 125 mg, from 125 mg to 175 mg, from 175 mg to 225 mg, or 50 mg to 300 mg, including all ranges and values therebetween. In some embodiments, the oral dosage form comprises 50 mg, 100 mg, 150 mg, or 200 mg of abemaciclib.

In some embodiments, a pharmaceutical combination is provided comprising an estrogen receptor (ER) degrader (e.g., compounds of Formula 1 disclosed herein) and a cyclin-dependent kinase (CDK) inhibitor in a single dosage form or in separate dosage forms. In some embodiments, the pharmaceutical combination is provided comprising an estrogen receptor (ER) degrader (e.g., compounds of Formula 1 disclosed herein) and a cyclin-dependent kinase (CDK) inhibitor are in separate dosage forms are administered by the same mode of administration or a different mode of administration. In some embodiments, the separate dosage forms of a pharmaceutical combination provided herein, are co-administered by simultaneous administration, sequential administration, overlapping administration, interval administration, continuous administration, or a combination thereof. In some embodiments, the dosage form is an oral dosage form. In some embodiments, the oral dosage for is a tablet or a capsule.

The following abbreviations have the definitions set forth below:

| | |
|---|---|
| ACN | Acetonitrile |
| AE | Adverse event |
| AcOH | Acetic acid |
| AESI | Adverse event of special interest |
| ALC | Absolute lymphocyte count |
| ALP | Alkaline phosphatase |
| ALT | Alanine aminotransferase |
| ANC | Absolute neutrophil count |
| AR | Adverse reaction |
| AST | Aspartate aminotransferase |
| AUC | Area under the concentration-time curve |
| BID | Twice daily |
| BOC | tert-butyloxycarbonyl |
| BP | Blood pressure |
| CBR | Clinical benefit rate |
| CD | Chimeric degrader |
| CDK | Cyclin-dependent kinase |
| CFR | Code of Federal Regulations |
| CI | Confidence interval |
| CK | Creatinine kinase |
| CR | Complete response |
| CT | Computed tomography |
| CTC | Circulating tumor cells |
| CTCAE | Common Terminology Criteria for Adverse Events |
| CuCN | copper cyanide |
| ctDNA | Circulating tumor DNA |
| DBU | 1,8-Diazabicyclo[5.4.0]undec-7-ene |
| DCM | dichloromethane |
| DCR | Disease Control Rate |
| DEA | Diethylamine |
| Development | Sarah Cannon Development Innovations |
| Innovations | |
| DLT | Dose-limiting toxicity |
| DMAC | dimethylacetamide |
| DMF | N,N-dimethylformamide |
| DOR | Duration of Response |
| EC | Ethics Committee |
| ECG | Electrocardiogram |
| ECHO | Echocardiogram |
| ECOG | Eastern Cooperative Oncology Group |
| eCRF | Electronic Case Report Form |
| eGFR | Estimated glomerular filtration rate |
| EOT | End of treatment (visit) |
| Et₃N | Triethylamine |
| EtOH | Ethanol |
| ER | Estrogen receptor |
| ER mut | Estrogen receptor 1 mutation at baseline |
| ESR | Estrogen receptor |
| ESR1 | Estrogen receptor gene 1 |
| FA | Formic acid |
| FAS | Full analysis set |
| FDA | Food and Drug Administration |
| FIH | First in human |
| FSH | Follicle-stimulating hormone |
| Ful | Fulvestrant |
| GCP | Good Clinical Practice |
| GLP | Good Laboratory Practice |
| HER2 | Human epidermal growth factor receptor 2 |
| HIPAA | Health Insurance Portability and Accountability Act |
| HNSTD | Highest non-severely toxic dose |
| HPLC | High performance liquid chromatography |
| HRMS | High resolution mass spectrometry |
| IB | Investigator's Brochure |
| IC50 | Half-maximal inhibitory concentration |
| ICF | Informed consent form |
| ICH | International Council for Harmonisation |
| IND | Investigational New Drug |
| INR | International normalized ratio |
| IRB | Institutional Review Board |
| ISF | Investigator Study File |
| IV | Intravenous |
| K₂CO₃ | potassium carbonate |
| LC/MS | Liquid chromatography-mass spectrometry |
| LVEF | Left ventricular ejection fraction |
| MedDRA | Medical Dictionary for Regulatory Activities |
| MeCN | acetonitrile |
| MRI | Magnetic resonance imaging |
| MTBE | Methyl tert-butyl ether |
| MTD | Maximum-tolerated dose |
| NaH₂PO₂ | sodium hypophosphite |
| NaCNBH₃ | sodium cyanoborohydride |
| NaBH(OAc)₃ | sodium triacetoxyborohydride |
| NCI CTCAE | National Cancer Institute Common Terminology |
| | Criteria for Adverse Events |
| NOAEL | No-observed-adverse-effect level |
| OR | Objective response |
| ORR | Overall response rate |
| OS | Overall survival |
| PD | Progressive disease |
| PDx | Pharmacodynamic |
| PET | Positron emission tomography |
| PFS | Progression-free survival |
| P-gp | P-glycoprotein |
| PGx | Pharmacogenetic |
| PHI | Protected health information |
| PhSO₃H | benzenesulfonic acid |
| Pic-BH₃ | 2-Picoline borane |
| PK | Pharmacokinetic |
| PO | Orally / by mouth |
| POI | Protein of interest |
| PR | Partial response |
| PT | Prothrombin time |
| PTT | Partial thromboplastin time |
| QD | Once daily |
| QT | ECG interval measured from the onset of the QRS |
| | complex to the end of the T wave |
| QTc | QT interval corrected for heart rate |
| RBC | Red blood cell count |
| RECIST | Response Evaluation Criteria in Solid Tumors |
| RP2D | Recommended Phase II Dose |
| RuPhos | 2-Dicyclohexylphosphino-2′,6′-diisopropoxybiphenyl |
| SAE | Serious adverse event |
| SAP | Statistical Analysis Plan |
| SAR | Suspected adverse reaction |
| SAS | Safety Analysis Set |
| SCRI | Sarah Cannon Research Institute |
| SD | Stable disease |
| SERD | Selective estrogen receptor degrader |
| SFC | Supercritical fluid chromatography |
| SRC | Safety Review Committee |
| SOCl₂ | thionyl chloride |
| STD₁₀ | Severely toxic dose in 10% of animals |
| SUSAR | Suspected unexpected serious adverse reaction |
| TEA | Triethylamine |
| TEAE | Treatment-emergent adverse event |
| TfOH | Trifluoromethanesulfonic acid |
| THF | tetrahydrofuran |
| TL | Target lesion |
| UAE | Unexpected adverse event |
| ULN | Upper limit of normal |
| v | Volume |

### Examples

The following examples are provided for illustrative purposes only and are not intended to limit the scope of the disclosure.

Compounds of Formula 1, including compound (1-a), compound (1-b), compound (1-c), compound (1-d), compound (1-e), compound (1-f), compound (1-g), compound (1-h), compound (1-i), compound (1-j), compound (1-k), and compound (1-1) can be prepared according to synthetic procedures analogous to those described in WO 2021/118629, the contents of which are incorporated herein by reference in their entirety.

For example, compound (1-g), compound (1-i), and compound (1-k) can be prepared as shown below.

### Example 1

### Synthesis of(S)-3-(6-fluoro-5-(4-((1-(2-fluoro-4-((3S,4R)-7-hydroxy-3-phenylchroman-4-yl)phenyl)piperidin-4-yl)methyl) piperazin-1-yl)-1-oxoisoindolin-2-yl) piperidine-2,6-dione (Compound (1-i))

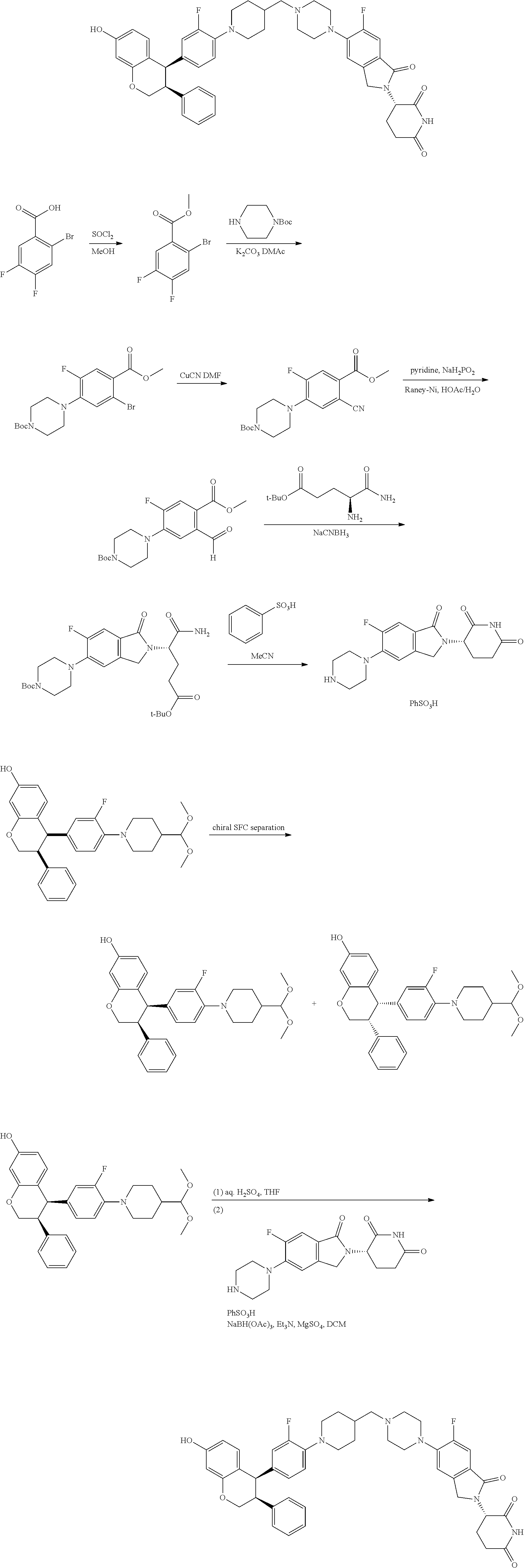

Step 1: Preparation of methyl 2-bromo-4,5-difluorobenzoate.

Thionyl chloride (130 g, 1.09 mol) was added slowly to a mixture of 2-bromo-4,5-difluorobenzoic acid (200 g, 0.84 mol) in MeOH (600 mL) at 10° C., the mixture was stirred at 80° C. for 3 h. TLC showed the reaction was completed. The mixture was cooled to room temperature, concentrated, then partitioned between ethyl acetate and water. The organic layer was washed with saturated Na₂CO₃ and brine twice, dried over Na₂SO₄ and concentrated to afford a crude methyl 2-bromo-4,5-difluorobenzoate (210 g, yield: 100%) which was used for the next step without further purification.

Step 2: Preparation of tert-butyl 4-(5-bromo-2-fluoro-4-(methoxycarbonyl)phenyl) piperazine-1-carboxylate.

A mixture of methyl 2-bromo-4,5-difluorobenzoate (210 g, 0.84 mol), tert-butyl piperazine-1-carboxylate (234 g, 1.25 mol) and K₂CO₃ (173 g, 1.25 mol) in N,N-dimethylacetamide (600 mL) was stirred at 80° C. for 16 h. TLC showed the reaction was completed. The mixture was added to water (2 L) and stirred for 10 min followed by the addition of ethyl acetate. The mixture was partitioned between ethyl acetate and water. The organic layer was washed with water, brine, dried over Na₂SO₄ and concentrated to afford tert-butyl 4-(5-bromo-2-fluoro-4-(methoxycarbonyl)phenyl) piperazine-1-carboxylate (315.8 g, yield: 90%).

Step 3: Preparation of tert-butyl 4-(5-cyano-2-fluoro-4-(methoxycarbonyl)phenyl) piperazine-1-carboxylate.

A mixture of tert-butyl 4-(5-bromo-2-fluoro-4-(methoxycarbonyl)phenyl) piperazine-1-carboxylate (306 g, 0.73 mol) and CuCN (98 g, 1.09 mol) in DMF (1.2 L) was stirred at 100° C. for 16 h. TLC showed the reaction was completed. The mixture was cooled to room temperature. Ethyl acetate (2 L) and ammonium hydroxide (2 L) were added and the mixture was stirred for 30 min. The mixture was filtered. The organic layer was washed with water, dried over Na₂SO₄ and concentrated to afford a crude product (254 g). This crude product was taken into petroleum ether (1 L) at reflux. The mixture was filtered and dried in oven at 50° C. to afford tert-butyl 4-(5-cyano-2-fluoro-4-(methoxycarbonyl)phenyl) piperazine-1-carboxylate (215 g, yield: 81%).

Step 4: Preparation of tert-butyl 4-(2-fluoro-5-formyl-4-(methoxycarbonyl)phenyl) piperazine-1-carboxylate.

To a solution of pyridine (391 g, 4.95 mol), water (200 mL), acetic acid (264 g, 4.4 mol) was added tert-butyl 4-(5-cyano-2-fluoro-4-(methoxycarbonyl)phenyl) piperazine-1-carboxylate (200 g, 0.55 mol) and Raney-nickel (85% in water, 100 g) at room temperature. The resulting mixture was heated to 60° C. Sodium hypophosphite (292 g in 500 mL water) was added dropwise into the mixture. The mixture was stirred for 16 h at 60° C. TLC showed the reaction not completed. The mixture was further stirred for 10 h. The mixture was cooled to room temperature. Ethyl acetate and water were added. The mixture was filtered. The organic layer was washed with water, IN HCl and brine, dried over Na₂SO₄ and concentrated under reduced pressure to afford a crude product (208 g, crude) which was further purified by silica-gel pad to provide 4-(2-fluoro-5-formyl-4-(methoxycarbonyl)phenyl) piperazine-1-carboxylate (86.5 g, yield: 43%).

Step 5: Preparation of tert-butyl(S)-4-(2-(1-amino-5-(tert-butoxy)-1,5-dioxopentan-2-yl)-6-fluoro-1-oxoisoindolin-5-yl) piperazine-1-carboxylate.

To a solution of tert-butyl 4-(2-fluoro-5-formyl-4-(methoxycarbonyl)phenyl) piperazine-1-carboxylate (81.5 g, 0.22 mol) in methanol (500 mL) was added tert-butyl(S)-4,5-diamino-5-oxopentanoate (54 g, 0.27 mol) at room temperature. Acetic acid (19.8 g, 0.33 mol) was added at 0° C. followed by the addition of sodium cyanoborohydride (27.6 g, 0.44 mol) slowly. The mixture was stirred at room temperature for 16 hours. TLC showed the reaction was completed. The mixture was concentrated and partitioned between ethyl acetate and water. The organic layer was washed with saturated citric acid, brine, dried over Na₂SO₄ and concentrated under reduced pressure to afford a crude product which was further purified by silica-gel pad to give tert-butyl(S)-4-(2-(1-amino-5-(tert-butoxy)-1,5-dioxopentan-2-yl)-6-fluoro-1-oxoisoindolin-5-yl) piperazine-1-carboxylate (80 g, yield: 69%).

Step 6: Preparation of(S)-3-(6-fluoro-1-oxo-5-(piperazin-1-yl) isoindolin-2-yl) piperidine-2,6-dione benzenesulfonic acid.

To a solution of(S)-4-(2-(1-amino-5-(tert-butoxy)-1,5-dioxopentan-2-yl)-6-fluoro-1-oxoisoindolin-5-yl) piperazine-1-carboxylate (67 g, 0.13 mol) in acetonitrile (670 mL) was added benzenesulfonic acid (43 g, 0.26 mol). The mixture was stirred at 80° C. for 16 h. LCMS showed the reaction was complete. The mixture was cooled to room temperature. The mixture was filtered and dried to afford(S)-3-(6-fluoro-1-oxo-5-(piperazin-1-yl) isoindolin-2-yl) piperidine-2,6-dione benzenesulfonic acid (56 g, 86%) as off-white solid. ¹H NMR (400 MHZ, DMSO-d6) δ 1.94-1.99 (m, 1H), 2.35-2.43 (m, 1H), 2.58-2.62 (m, 1H), 2.88-2.91 (m, 1H), 3.30 (br s, 8H), 4.38 (d, J=17.2 Hz, 1H), 4.26 (d, J=17.2 Hz, 1H), 5.08 (dd, J=13.2, 5.2 Hz, 1H), 7.29-7.35 (m, 4H), 7.49 (d, J=8.7 Hz, 1H), 7.60 (m, 2H), 8.72 (s, 2H), 10.99 (s, 1H). LCMS m/z 347.3 [M+1]⁺.

Step 7: Preparation of (3S,4R)-4-(4-(4-(dimethoxymethyl)piperidin-1-yl)-3-fluorophenyl)-3-phenylchroman-7-ol.

The racemic cis-4-(4-(4-(dimethoxymethyl)piperidin-1-yl)-3-fluorophenyl)-3-phenylchroman-7-ol (50.0 g, 104 mmol, prepared according to the procedure described in WO 2021/118629) was separated by chiral SFC (column: DAICEL CHIRALCEL OD (250 mm×30 mm, 10 um); mobile phase: 0.1% NH₃H₂O in MeOH; as phase B; B %: 60%-60%). The first fraction collected provided (3R,4S)-4-(4-(4-(dimethoxymethyl)piperidin-1-yl)-3-fluorophenyl)-3-phenylchroman-7-ol (15.0 g, 99.4% purity) as an off-white solid. [α]_{D}²⁵=335.8 (1 g/100 mL in EtOAc); LCMS m/z 478.2 [M+1]⁺; ¹H NMR (400 MHZ, DMSO-d6) δ 9.34 (s, 1H), 7.12-7.23 (m, 3H), 6.65-6.86 (m, 4H), 6.25-6.35 (m, 3H), 6.13 (d, 1H), 4.30 (t, 1H), 4.23 (m, 2H), 4.07 (d, J=6.4 Hz, 1H), 3.53 (m, 1H), 3.25 (s, 6H), 3.15-3.24 (m, 2H), 2.42-2.50 (m, 2H), 1.57-1.72 (m, 3H), 1.22-1.40 (m, 2H). The second fraction collected provided (3S,4R)-4-(4-(4-(dimethoxymethyl)piperidin-1-yl)-3-fluorophenyl)-3-phenylchroman-7-ol (16.0 g, 98.1% purity) as a brown solid. [α]_{D}²⁵=−303.9 (0.5 g/100 mL in EtOAc); LCMS m/z 478.2 [M+1]⁺; ¹H NMR (400 MHz, DMSO-d6) δ 9.45 (br s, 1H), 7.16 (m, 3H), 6.65-6.80 (m, 4H), 6.25-6.32 (m, 3H), 6.13 (d, J=13.6 Hz, 1H), 4.32 (t, 1H), 4.17-4.27 (m, 2H), 4.07 (d, J=6.4 Hz, 1H), 3.55 (m, 1H), 3.25 (s, 6H), 3.16-3.25 (m, 2H), 2.40-2.50 (m, 2H), 1.57-1.72 (m, 3H), 1.22-1.37 (m, 2H).

Step 8: Preparation of(S)-3-(6-fluoro-5-(4-((1-(2-fluoro-4-((3S,4R)-7-hydroxy-3-phenylchroman-4-yl)phenyl)piperidin-4-yl)methyl) piperazin-1-yl)-1-oxoisoindolin-2-yl) piperidine-2,6-dione (Compound (1-i)).

The separated (−)-enantiomer from step 7 was first deprotected under the acidic condition and then reacted with the product from step 6 under the same condition as described in WO 2021/118629 to provide compound (1-i). LCMS m/z 762.2 [M+H]⁺; ¹H NMR (400 MHZ, DMSO) δ 10.99 (s, 1H), 9.35 (s, 1H), 7.43 (d, J=11.6 Hz, 1H), 7.27-7.16 (m, 4H), 6.82-6.72 (m, 3H), 6.68 (d, J=8.2 Hz, 1H), 6.35-6.28 (m, 3H), 6.15 (d, J=14.1 Hz, 1H), 5.08 (dd, 1H), 4.43-4.18 (m, 5H), 3.63-3.53 (m, 1H), 3.32-3.30 (m, 2H), 3.26-3.20 (m, 2H), 3.12 (br s, 2H), 2.95-2.85 (m, 1H), 2.63-2.48 (m, 7H), 2.42-2.32 (m, 1H), 2.22 (br d, 2H), 2.02-1.91 (m, 1H), 1.81-1.73 (m, 2H), 1.70-1.61 (m, 1H), 1.27-1.20 (m, 2H); HRMS calculated for C₄₄H₄₅F₂N₅O₅ exact mass 761.3389, observed [M+1]⁺762.3593.

### Example 2

### Synthesis of(S)-3-(5-(4-((1-(2-fluoro-4-((3S,4R)-7-hydroxy-3-phenylchroman-4-yl)phenyl) piperidin-4-yl)methyl) piperazin-1-yl)-1-oxoisoindolin-2-yl) piperidine-2,6-dione (Compound (1-g))

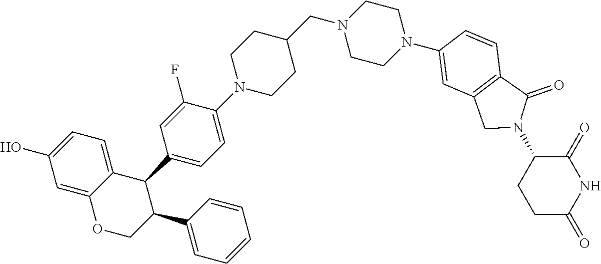 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

The preparation of compound (1-g) was carried out using the same method as for the preparation of compound (1-i), which was also described in WO 2021/118629. LCMS m/z 743.7 [M+H]⁺; ¹H NMR (400 MHZ, DMSO) δ 10.97 (s, 1H), 9.37 (s, 1H), 7.52 (d, J=8.2 Hz, 1H), 7.17 (m, 3H), 7.07 (s, 1H), 7.06 (d, J=8.2 Hz, 1H), 6.84-6.71 (m, 3H), 6.69 (d, J=8.3 Hz, 1H), 6.37-6.27 (m, 3H), 6.16 (br d, J=12 Hz, 1H), 5.06 (dd, J=12.5 Hz, 4.2 Hz, 1H), 4.40-4.18 (m, 5H), 3.57 (m, 1H), 3.32-3.17 (m, 6H), 2.96-2.87 (m, 1H), 2.60-2.48 (m, 7H), 2.40-2.33 (m, 1H), 2.22 (br d, 2H), 2.00-1.90 (br d, 1H), 1.80-1.75 (m, 2H), 1.70-1.60 (m, 1H), 1.28-1.20 (m, 2H); HRMS calculated for C₄₄H₄₆FN₅O₅ exact mass 743.3483, observed [M+1]⁺744.3567.

### Example 3

### Synthesis of(S)-3-(5-(4-((1-(4-((3S,4R)-7-hydroxy-3-phenylchroman-4-yl)phenyl)piperidin-4-yl)methyl) piperazin-1-yl)-1-oxoisoindolin-2-yl) piperidine-2,6-dione (Compound (1-k))

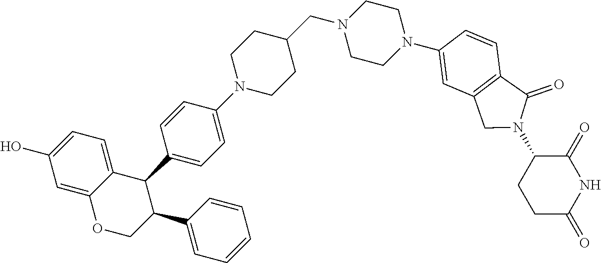 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

The preparation of compound (1-k) was carried out using the same method as for the preparation of Compound (1-i). The synthesis of the chiral chroman building block followed the procedure described in WO 2021/118629. LCMS m/z 725.7 [M+H]⁺; ¹H NMR (400 MHZ, DMSO) δ 10.98 (s, 1H), 9.31 (s, 1H), 7.53-7.51 (m, 1H), 7.25-7.02 (m, 5H), 6.77 (m, 2H), 6.73-6.60 (m, 3H), 6.40 (d, J=8.3 Hz, 2H), 6.33-6.25 (m, 2H), 5.06 (dd, 1H), 4.39-4.31 (m, 2H), 4.26-4.17 (m, 3H), 3.64-3.50 (m, 3H), 3.28-3.25 (br s, 4H), 2.94-2.89 (m, 1H), 2.64-2.48 (m, 7H), 2.41-2.33 (m, 1H), 2.19 (br, 2H), 2.00-1.93 (m, 1H), 1.86-1.60 (m, 3H), 1.29-1.12 (m, 2H); HRMS calculated for C₄₄H₄₇N₅O₅ exact mass 725.3577, observed [M+1]⁺726.3675.

### Example 4

### Synthesis of (R)-3-(5-(4-((1-(2-fluoro-4-((3S,4R)-7-hydroxy-3-phenylchroman-4-yl)phenyl)piperidin-4-yl)methyl) piperazin-1-yl)-1-oxoisoindolin-2-yl) piperidine-2,6-dione (Compound (1-h))

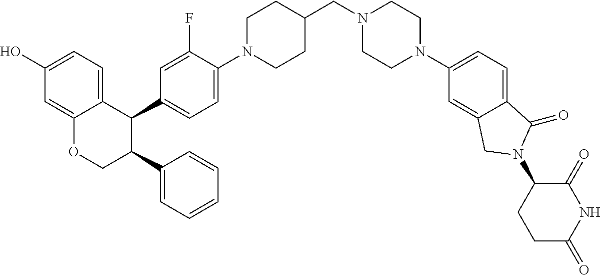 [SMILES: O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

The preparation of compound (1-h) was carried out using the same method as described for the preparation of Compound (1-i) but for the use of tert-butyl(R)-4,5-diamino-5-oxopentanoate instead of tert-butyl(S)-4,5-diamino-5-oxopentanoate (see Step 5 in Example 1). The crude product was first purified by column chromatography (SiO₂, DCM/MeOH=1/0 to 10/1), and then further purified by SFC (column: REGIS (s,s) WHELK-01 (250×50 mm, 10 μm); mobile phase B: (IPA/ACN=1/1); B %: 65%-65% over 2.7 min). (R)-3-(5-(4-((1-(2-fluoro-4-((3S,4R)-7-hydroxy-3-phenylchroman-4-yl)phenyl)piperidin-4-yl)methyl) piperazin-1-yl)-1-oxoisoindolin-2-yl) piperidine-2,6-dione (compound (1-h)) was obtained as a white solid. Chemical purity: 99.4% (detected at 220 nm); 99.5% (detected at 254 nm). Chiral purity (for glutarimide stereogenic center): 98.7% (analyzed by SFC using (S,S)-WELK-01 as a chiral column). LCMS m/z 744.5 [M+H]⁺; ¹H NMR (400 MHZ, DMSO) δ 11.00 (s, 1H), 9.39 (s, 1H), 7.58 (d, J=8.4 Hz, 1H), 7.23 (s, 3H), 7.05-7.17 (m, 2H), 6.85-6.73 (m, 4H), 6.38-6.34 (m, 3H), 6.19 (br d, J=14.0 Hz, 1H), 5.09 (br dd, J=4.8, 13.2 Hz, 1H), 4.41-4.24 (m, 5H), 3.67-3.55 (m, 1H), 3.38-3.27 (m, 8H), 3.05-2.87 (m, 1H), 2.75-2.58 (m, 4H), 2.50-2.35 (m, 2H), 2.27 (br d, J=6.8 Hz, 2H), 2.11-1.94 (m, 1H), 1.84 (br, 2H), 1.70 (s, 1H), 1.29-1.27 (m, 2H). HRMS calculated for C₄₄H₄₆FN₅O₅ exact mass 743.3483, observed [M+1]⁺744.3555.

### Example 5

### Preparation of a Mixture of Compound (1-g) and Compound (1-h)

Compound (1-g) may be mixed with compound (1-h) to prepare a mixture having a desired ratio of compound (1-g) to compound (1-h).

First, the chiral purity of a batch of compound (1-g) is analyzed by analytical chiral SFC to determine the trace amount of compound (1-h). Then the chiral purity of a batch of compound (1-h) is also analyzed by analytical chiral SFC to determine the trace amount of compound (1-g). Based on the chiral purity analysis results, the amount needed for each of compound (1-g) and compound (1-h) to prepare a mixture of compound (1-g) and compound (1-h) of the desired ratio can be calculated. The following is an example of making a mixture of compound (1-h) and compound (1-g) as a batch for preclinical toxicology studies.

A batch of compound (1-g) was produced and chiral SFC analysis showed a ratio of 99.2%/0.8% for (1-g)/(1-h). A batch of compound (1-h) was also produced and chiral SFC analysis showed a ratio of 99.5%/0.5% for (1-h)/(1-g). To make a mixture having a (1-g)/(1-h) ratio of 98.1%/1.9% and the total amount of 638 g, the amount of compound (1-g) and compound (1-h) needed to make the mixture was determined. To calculate the amount of compound (1-g) and compound (1-h), the following equation was used where Y stands for the amount in grams needed for (1-h), and the amount needed for (1-g) was (638-Y) grams:

$$[ ( 638 - Y ) \times 99.2 \% + Y \times 0.5 ] / [ ( 638 - Y ) \times 0.8 \% + Y \times 99.5 ] = 98.1 \% / 1.9 \%$$

After solving the above equation, Y was found to be 7.3 g. Therefore, 7.3 g of the batch of compound (1-h) was accurately weighed and was then mixed well with 630.7 g of the batch of compound (1-g) to arrive at the (1-g)/(1-h) ratio of 98.1%/1.9%. This mixture in a total amount of 638 g was used in preclinical toxicology studies.

The same method can be used to calculate the amount of compound (1-g) and compound (1-h) needed to prepare a mixture compound (1-g) and compound (1-h) having a desired ratio. Likewise, the same method can be used to prepare mixtures of compound (1-e) and compound (1-f); compound (1-i) and compound (1-j); and compound (1-k) and compound (1-k), or mixtures of any of the compounds of Formula 1 described herein having a desired ratio.

### Example 6—Pre-clinical Data for Compound (1-g)

Compound (1-g) binds to human ERa with an inhibitor constant (Kᵢ) of 0.53 nM and a half-maximal inhibitory concentration (IC₅₀) of 6.02 nM (4.4 ng/mL) and induces rapid ERa degradation in multiple ERa-positive breast cancer cell lines, such as MCF7.

### Cell Growth Inhibition of MCF7 Cells by Compound (1-g) and Compound (1-h)

MCF7 cells were obtained from American Type Culture Collection (ATCC). For cell growth assay, MCF7 cells were seeded in 96-well plates at 2000 cells/well in 90 μL of DMEM medium containing 10% fetal bovine serum, and then incubated at 37° C. with 5% CO₂ in air overnight. The following day, compound (1-g) and compound (1-h) were administered to the cells by using 1000× compound stock solution prepared in DMSO at concentrations of 3, 1, 0.33, 0.11, 0.037, 0.012, 0.0041, 0.0013, and 0.00046 mM. 1000x compound stock solution was first diluted in culturing medium to 10×, then 10 μL compound medium was added to each well in the cell plates. After administration of compound (1-g) and compound (1-h), the cells were then incubated at 37° C. for 6 days. Upon completion, the plates were equilibrated at room temperature for approximately 10 minutes. 100 μL of CellTiter-Glo® Reagent (Promega) was added to each well. The plates were then incubated at room temperature for 10 minutes and luminescence was recorded by EnSpire plate reader (PerkinElmer).

### Results

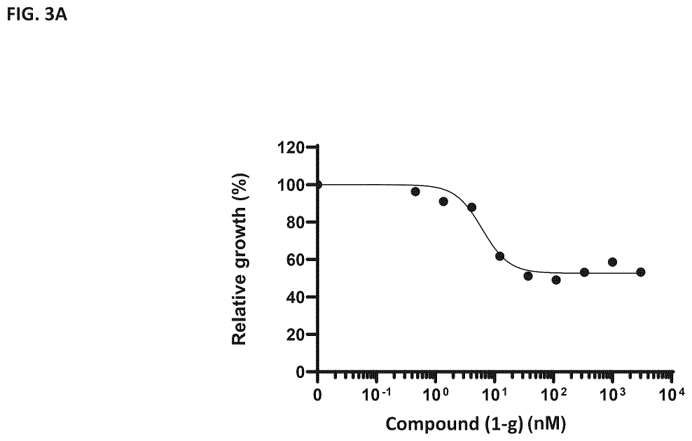

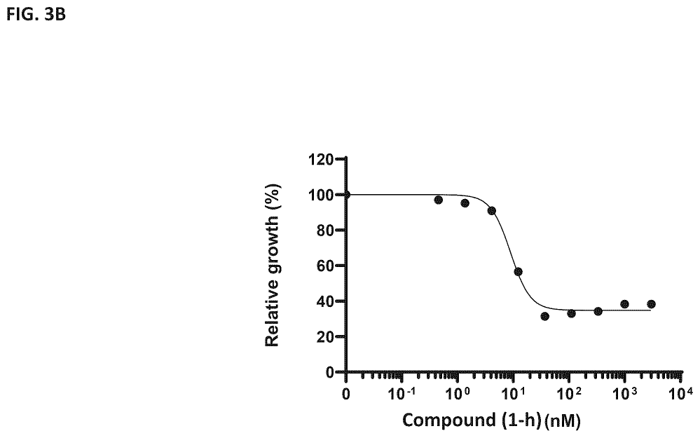

To access the degradation of ER induced by compound (1-g), MCF7 cells were treated with compound (1-g). FIG. 3A depicts the growth inhibitory effects demonstrated by compound (1-g) in a luminescence-based MCF7 cell proliferation assay. As shown in FIG. 3A, compound (1-g) reduced the tumor growth by about 50%. The GI₅₀ value of compound (1-g) was found to be 6.2 nM.

To access the degradation of ER induced by compound (1-h), MCF7 cells were treated with compound (1-h). FIG. 3B depicts the growth inhibitory effects demonstrated by compound (1-h) in a luminescence-based MCF7 cell proliferation assay. As shown in FIG. 3B, compound (1-h) reduced the tumor growth by about 62%. The GI₅₀ value of compound (1-h) was found to be 9.0 nM. These data demonstrate that compound (1-g) and compound (1-h) display potent anti-tumor activity against a well-established ER-positive breast cancer model, concurrent with robust degradation of ER in the tumors.

The growth inhibitory effects of compound (1-g) and compound (1-h) are presented in Table 2.

TABLE 2
The growth inhibitory effects of compound (1-g) and compound (1-h)

| Compound | Structure | GI₅₀ [nM] |
|---|---|---|
| 1-g | 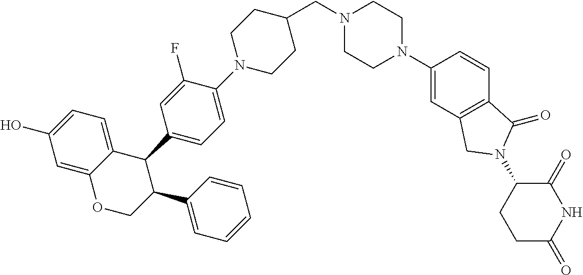 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] | 6.2 |
| 1-h | 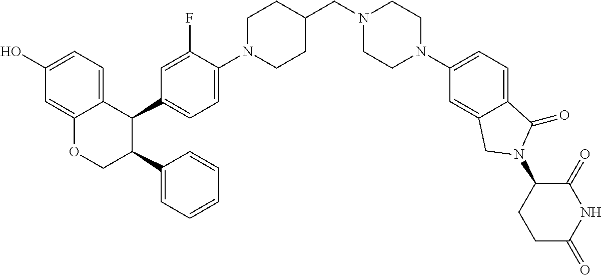 [SMILES: O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] | 9.0 |

### Oral Bioavailability of Compound (1-g) in Different Subjects

The efficacy of oral administration of compound (1-g) against xenografted tumors was demonstrated in 2 studies in mice involving once daily oral administration of 30 to 120 mg/kg of compound (1-g). Once daily oral administration of 30 mg/kg/day (the lowest dose level evaluated) achieved tumor regression in MCF7 xenograft tumors in mice. In a tamoxifen-resistant MCF7 xenograft model, enhanced anti-tumor activity was observed when compound (1-g) was administered in combination with the CDK4/6 inhibitor palbociclib. These in vivo pharmacology studies suggest that compound (1-g) may be able to treat tamoxifen-resistant tumors.

Following repeat oral administration in the GLP-compliant toxicology studies, compound (1-g) exposure in terms of AUC₀₋₂₄ₕ increased in an approximately dose-dependent manner in both rats and dogs; however, exposure was slightly less than dose proportional across the dose range of 5 to 45 mg/kg/day in dogs following repeat administration. There were no marked sex-based differences in exposure, but in the 28-day toxicity study of compound (1-g) in rats, exposures were 1.4 to 1.9-fold higher in female rats compared to males. In dogs, exposures to compound (1-g) at 45 mg/kg/day decreased in females and slightly decreased in males following repeat dosing. No significant accumulation of compound (1-g) was reported with repeated dosing in both preclinical species.

Following oral administration of compound (1-g) to female rats, the highest exposures were observed in the large and small intestine with limited exposure to the brain. Compound (1-g) was quantifiable in mammary gland tissue (i.e., the expected pharmacological site of action) and the ovaries and uterus (2 target tissues observed in toxicology studies). Observed exposures in the evaluated tissues (based on area under the concentration-time curve [AUC]) were greater than that observed in plasma (2,800 ng.h/mL).

Compound (1-g) was weakly metabolized by human cytochrome P450 CYP2C8, CYP3A4, and CYP3A5 in vitro. Compound (1-g) inhibited CYP2B6, CYP2C9, and CYP2C19 by 48.0%, 41.1%, and 39.1% at the highest concentration of 10 μM, respectively, in the presence of NADPH, with a reported IC₅₀>10 μM, without showing time-dependent inhibition. Compound (1-g) displayed weak time-dependent inhibition against CYP3A with midazolam as the substrate (31.3% inhibition at a concentration of 10 μM). Compound (1-g) did not directly inhibit CYP1A2, CYP2C8, CYP2D6 or CYP3A at the highest concentration evaluated (10 μM). Compound (1-g) did not significantly induce activity or mRNA expression of CYP2B6, or CYP3A4 in vitro, but increased CYP1A2 mRNA expression by 1.4- and 3.1-fold in 1 of 3 hepatocyte donors at 0.5 μM (i.e., 372 ng/mL) and 2 μM, while no induction of CYP1A2 enzyme activity was observed.

In the 28-day GLP toxicity studies of compound (1-g) in rats and dogs, key organ effects were observed in both species and the effects were considered on target pharmacological effects.

Overall, there were no off-target adverse toxicities observed. In the 28-day oral toxicity study of compound (1-g) in rats, the severely toxic dose in 10% of animals (STD₁₀) was considered to be 270 mg/kg/day in females. The 270 mg/kg/day dose level was considered the no-observed-adverse-effect-level (NOAEL) in male rats due to the absence of any adverse findings. In the 28-day oral toxicity study of compound (1-g) in dogs, the highest non-severely-toxic dose (HNSTD) was considered to be 45 mg/kg/day due to the presence of pharmacological effects on the reproductive organs of both sexes.

A human starting dose of 261 mg/day was calculated based on 1/10th the STD10 value in rats from 28-day GLP toxicity studies, and a human starting dose of 250 mg/day was calculated from ⅙^{th} of the HNSTD determined in the 28-day GLP toxicity studies in dogs. However, the proposed starting dose in this clinical study is 100 mg/day which provides an additional 2.5-fold safety margin.

### Example 7-Phase 1 Study of Compound (1-g) in Patients with ER+/HER2− Locally Advanced or Metastatic Breast Cancer

a. Study Rationale

ER plays a critical role in breast cancer initiation and proliferation. Modulation of estrogen and estrogen activity is standard of care (SOC) therapy for patients with ER-positive breast cancer. Compound (1-g) is a chimeric degrader that targets and degrades the protein of interest (POI), ERa, without activating an ER signal. Compound (1-g) contains both ligands of human ERa and E3 ligase and effectively brings human ERa to proximity of E3 ligase, which subsequently induces robust human ERa ubiquitination and degradation.

b. Study Description

The study employed an open-label, 3+3 design, with the option of adding backfill patients to each cohort cleared and deemed safe. Patients must have had locally advanced or metastatic ER+/HER2− breast cancer and received at least 2 prior lines of endocrine treatment, or at least 1 prior line if combined with a CDK4/6 inhibitor. Compound (1-g) was administered orally once daily in 28-day cycles. Tumor responses were assessed every 2 cycles using RECIST v1.1 criteria. Therefore, the initial dose of 100 mg QD was chosen to limit the number of patients exposed to lower efficacious dose levels, while adding an additional 2.5-fold safety margin. The study also involves an ongoing dose escalation study where participants receive 100 mg, 200 mg, 300 mg, 400 mg, and 600 mg once daily (QD) dose levels.

c. Objectives

The primary and secondary objectives of the study included:
evaluating the safety and tolerability of compound (1-g);
evaluating the preliminary anti-tumor activity of compound (1-g);
characterizing the pharmacokinetic (PK) profile of a single dose and after multiple doses of compound (1-g);
evaluating the pharmacodynamic (PDx) effect of compound (1-g) in ERa degradation;
evaluating the relationship between circulating tumor cells (CTCs) count and administration of compound (1-g);
evaluating the relationship between estrogen receptor gene 1 (ESR1) mutation status and anti-tumor activity of compound (1-g); and
evaluating the relationship between circulating tumor DNA (ctDNA) levels and anti-tumor activity of compound (1-g).
   d. Patient eligibility

Patients who meet the following criteria were considered eligible to participate in the clinical study.

Inclusion criteria:
Written informed consent, according to local guidelines, signed and dated prior to the performance of any study-specific procedures, sampling, or analyses;
Adult male and female patients, at least 18 years-of-age at the time of signature of the informed consent form (ICF;)
Female participants must meet one of the following criteria described in a) or b):
a) Premenopausal or perimenopausal women must receive concurrent treatment with a luteinizing hormone-releasing hormone (LHRH) agonist beginning at least 4 weeks before the start of trial therapy and agree to continue the LHRH agonist throughout the duration of study treatment, have a negative serum pregnancy test within 7 days of initiating treatment, and agree to follow guidelines for use of highly effective contraception as outlined in Appendix C during the study and for 90 days following the last dose of study drug.
b) Postmenopausal women must be defined by as at least one of the following:
   i. Age≥60, or
   ii. Spontaneous amenorrhea (i.e., in the absence of chemotherapy, tamoxifen, toremifene, or ovarian suppression) for ≥12 months following cessation of all exogenous hormonal treatment, or
   iii. 6 months of spontaneous amenorrhea with serum follicle-stimulating hormone (FSH) levels and an estradiol value in the post-menopausal range per institutional standards, or
   iv. Prior bilateral oophorectomy performed at least 6 weeks before screening, with or without hysterectomy.
Eastern Cooperative Oncology Group (ECOG) Performance Status score of 0 or 1;
Patients with a confirmed diagnosis of advanced, unresectable, and/or metastatic breast cancer following disease progression on standard treatment, or for whom no therapy of proven efficacy exists, or who are not amenable to standard therapies;
Patient has a histologically and/or cytologically confirmed diagnosis of estrogen-receptor positive (ER+) breast cancer;
Patient with HER2-negative breast cancer as defined by American Society of Clinical Oncologists/College of American Pathologists (ASCO/CAP) guidelines (Wolff et al., Arch. Pathol. Lab. Med. 2018; 142:1364-82);
Patients must have received at least 2 prior endocrine regimens in any setting (neoadjuvant, adjuvant or advanced/metastatic) or at least 1 prior line of endocrine therapy if combined with CDK4/6 inhibitor;
Prior chemotherapy is not required, but up to 3 prior regimens of cytotoxic chemotherapy will be allowed in the locally advanced/metastatic setting;
Patients must have at least 1 measurable lesion according to RECIST Version 1.1 or at least 1 predominantly lytic bone lesion in the absence of measurable disease;
Acceptable organ function, as evidenced by the following laboratory data:
   Renal function, as follows:
   Creatinine clearance of ≥60 mL/min by the Cockcroft-Gault equation or equivalent;
   Liver function as follows:
   Total bilirubin≤1.5×upper limit of normal (ULN [≤5×ULN for patients with known Gilbert's syndrome]);
   Aspartate aminotransferase (AST)≤2.5×ULN or ≤5×ULN in the presence of liver metastases;
   Alanine aminotransferase (ALT)≤2.5×ULN or ≤5×ULN in the presence of liver metastases;
   International normalized ratio (INR)≤2;
Acceptable hematologic function:
Hemoglobin≥9 g/dL;
Absolute neutrophil count (ANC)≥1,000 cells/mm³;
Platelet count ≥75,000 cells/mm³;
Male patients with female partners of childbearing potential are required to use two forms of acceptable contraception, including one barrier method, during their participation in the study and for 90 days following last dose. Male patients must also refrain from donating sperm during their participation in the study and for 90 days following last dose.
Life expectancy ≥12 weeks after the start of the treatment.
Backfill patient must have available historical testing to confirm ESR1 mutations and must meet all inclusion criteria listed here.

### Exclusion Criteria

Treatment with any of the following:
   Any cytotoxic chemotherapy, investigational agents, or other anti-cancer drugs for the treatment of locally advanced or metastatic breast cancer within 14 days prior to the first administration of compound (1-g);
   >3 prior chemotherapy regimens for locally advanced or metastatic breast cancer;
   Radiation therapy within 14 days prior to first study drug administration that did not resolve to tolerable toxicity, or prior irradiation to >25% of bone marrow. Prior palliative radiotherapy to metastatic lesion(s) is permitted, provided it has been completed 7 days prior to study enrollment and no clinically significant toxicities are expected (e.g., mucositis, esophagitis).

Major surgery within 21 days prior to the first study drug administration (exception: patients may enroll if fully recovered or without intolerable or clinically significant adverse effects, but at least 14 days must have elapsed between major surgery and first study drug administration).

Use of prophylactic growth factors and blood transfusions≤14 days prior to the first study drug administration.

Proton pump inhibitors should not be used within at least 48 hours prior to C1D1 and are prohibited during the study.
With the exception of alopecia and ≤Grade 2 peripheral neuropathy, any unresolved toxicities from prior therapy greater than Common Terminology Criteria for Adverse Events (CTCAE) Grade 1 at the time of starting study treatment. Note: Patients with chronic Grade 2 toxicities that are asymptomatic or adequately managed with stable medication may be eligible with approval by the Medical Monitor and/or Sponsor.
Known symptomatic brain metastases requiring the use of systemic corticosteroids ≥10 mg/day prednisone or equivalents. Asymptomatic and treated, or asymptomatic untreated brain metastases are allowed as long as patients are clinically stable. Stable doses of anticonvulsants are allowed.
Any condition that impairs a patient's ability to swallow whole pills. Impairment of gastrointestinal function (GI) or GI disease or other condition at baseline that will interfere significantly with the absorption, distribution, or metabolism of compound (1-g) (e.g., ulcerative disease, uncontrolled nausea, vomiting, diarrhea Grade ≥2, malabsorption syndrome).
Any of the following cardiac criteria currently or within the last 6 months:
Mean resting corrected QT interval (QTc)>470 msec within 28 days prior to the first administration of study drug;
Any clinically important abnormalities (as assessed by the Investigator) in rhythm, conduction, or morphology of resting electrocardiograms (ECGs), e.g., complete left bundle branch block, third-degree heart block;
Congestive heart failure (New York Heart Association Class II-IV);
Any factors that increase the risk of QTc prolongation or risk of arrhythmic events such as heart failure, acute hypokalemia, congenital long QT syndrome, family history of long QT syndrome or unexplained sudden death under 40 years-of-age, or any new concomitant medication known to prolong the QT interval;
Patients with a left ventricular ejection fraction (LVEF)<50% or the lower limit of normal of the institutional standard within 28 days prior to the first administration of study drug will be excluded.
Any evidence of severe or uncontrolled systemic diseases, including uncontrolled hypertension, uncontrolled diabetes mellitus, and any serious active infection requiring systemic treatments. Screening for chronic conditions is not required.
HIV infection with a current or a history of AIDS-defining illness or HIV infection with a CD4+ T cell count <350 cells/μL and an HIV viral load more than 400 copies/μL.
Patients with active viral (any etiology) hepatitis are excluded. However, patients with serologic evidence of chronic HBV infection (defined by a positive hepatitis B surface antigen test and a positive anti-hepatitis core antigen antibody test) who have a viral load below the limit quantification (HBV DNA titer <1000 cps/mL or 200 IU/mL) and are not currently on viral suppressive therapy may be eligible and should be discussed with the Medical Monitor. Patients with a history of HCV infection who have completed curative antiviral treatment and have a viral load below the limit of quantification may be eligible.
Diagnosis of other active invasive cancers other than the one treated in this study within 2 years prior to first study drug administration; Exceptions include appropriately treated basal cell or squamous cell skin cancer, or in situ carcinoma of uterine cervix, or other local tumors considered cured by local treatment.
Prior history of allergic reaction to the composition/excipients of compound (1-g).
Psychological, familial, sociological, or geographical conditions that do not permit compliance with the protocol and/or follow-up procedures.
   e. Treatment Plan

All patients received compound (1-g) orally. The time of day for administration of compound (1-g) was consistent. Compound (1-g) was taken whole with liquid(s) and with food in the morning. The patient avoided taking other medications at the same time as compound (1-g). When possible, the patient took other medications in the evening or separated by a minimum of 4 hours following compound (1-g) dosing. On scheduled PK collection days (Cycle 1 Day 1, Cycle 1 Day 15, and Day 1 of Cycles 2, 3, 4, and 6) patients were instructed to wait until they arrive at the study center to take their study drug when told. If a patient missed a dose of compound (1-g), the patient was required to take the dose as soon as possible, but not less than 12 hours before the next dose is due. If vomiting occurred after taking the study treatment, the patient was instructed not to retake the dose and record the dose recorded as taken in the dosing diary, noting that the patient vomited and marking the time of vomiting. Patients were instructed to take the next scheduled dose of compound (1-g).

f. End of Study Treatment (EOT)

Patients were discontinued from study treatment for any of the following reasons:
objective disease progression (Patients who are receiving clinical benefit in the opinion of the treating Investigator may be allowed to stay on study treatment after consultation with the Medical Monitor and Sponsor Representative);
intolerable toxicity that are irreversible thought to be related to compound (1-g);
treatment discontinuation criteria as per dose modification criteria;
conditions requiring therapeutic intervention not permitted by the protocol;
intercurrent illness interfering with the patient's ability to follow study requirements (treatment or assessments);
another condition leading to patient's inability to comply with study requirements;
patient withdraws consent from study treatment or study participation altogether;
start of a new anti-cancer therapy;
patient's death; and
study termination.

After discontinuation from protocol treatment, patients must be followed for adverse events (AEs) for 30 days after their last dose of study drug. All new AEs occurring during the 30-days following termination of treatment must be reported and followed until resolution or stabilization unless, in the opinion of the Investigator, these values are not likely to improve because of the underlying disease.

All patients who have Grade 3 or 4 laboratory abnormalities (per National Cancer Institute Common Terminology Criteria for Adverse Events at the time of discontinuation must be followed until the laboratory values have returned to Grade 1 or 2 unless it is, in the opinion of the Investigator, not likely that these values are to improve and/or are the results of underlying diseases.

g. Concomitant Medications

The following concomitant medications and therapies are permitted in the course of the study:
bisphosphonate use, as recommended according to practice guidelines;
receptor activator of nuclear factor kappa-B ligand (RANKL) inhibitor use, as recommended according to practice guidelines;
growth factors and blood transfusions will be permitted after Cycle 1;
palliative radiotherapy is permitted; and
CYP inhibiting and CYP inducing agents are allowed in the study.

Medicinal products that alter the pH of the upper GI tract may alter the solubility of compound (1-g), expected to impact and limit its bioavailability. Such agents include, but are not limited to, proton pump inhibitors (e.g., omeprazole), H2-antagonists (e.g., ranitidine) and antacids. Proton pump inhibitors are prohibited. For patients who require mandatory use of antacid medication, proton pump inhibitors must be replaced with H₂-antagonists or antacids. If H2 antagonist or antacid is used, study drug should be taken >2 hours before or 10 hours after dosing with H₂-antagonists. Antacids, locally-acting acid neutralizing agent intake, are to be separated from study drug doses by 2 hours.

The following concomitant medications and therapies are prohibited in the course of the study:
no other investigational therapy should be given to patients;
no anticancer agents other than the study treatments should be given to patients; if such agents are required for a patient, then the patient must first be withdrawn from the study;
anticoagulation with coumarin-derivatives is not permitted; however, the use of anticoagulants to keep the port line patent will be permitted;
proton pump inhibitors are prohibited due to their long PD effect; this type of drugs should be replaced with H₂-antagonists or antacids; and
any new or concomitant QT prolonging medications within the last 6 months.
   h. Study Assessment and Evaluation

The key procedures required in this study include: reporting of all AEs occurring after the ICF has been signed; PK samples throughout the study; baseline and on-treatment blood biomarker assessments; tumor biopsy biomarker assessments; tumor assessments (based on CT/positron emission tomography [PET], bone scans, and/or magnetic resonance imaging [MRI] scan) according to RECIST Version 1.1.

A cycle of treatment was scheduled to last 4 weeks (28 calendar days). Priority should be given to PK collection at the time specified. Vital signs and ECG assessments should be performed prior to specimen collections or with proper time interval in between as to not artificially alter the vital signs collection.

i. Assessments During Study Treatment

Assessments during study treatment included the following parameters:
physical examination;
ECOG performance status;
vital signs (including weight);
hematology (including ALC, ANC, RBC, reticulocytes, hemoglobin, hematocrit, 5-part differential, platelet counts, and PT/PTT/INR);
biochemistry (sodium, potassium, phosphate, chloride, creatinine, total calcium, total CO₂ or venous HCO₃, albumin, total protein, AST, ALT, ALP, total bilirubin, direct bilirubin, lactate dehydrogenase, glucose, CK, urea nitrogen, and uric acid);
urinalysis;
pharmacokinetics; and
tumor biopsy.
   j. Pharmacokinetic Assessments

The plasma PK parameters (including AUC_{(0-∞)}, AUC_{(0-τ)}, Cₘₐₓ, tₘₐₓ, and T_{1/2}) of compound (1-g) following oral administration was assessed by analysis of blood samples. During treatment, PK blood samples were taken at approximately the following time points:
Cycle 1 Day 1, and Day 15: pre-dose; at 1, 2, and 4-hour post-dose (±5 min); 6 and 8-hour post-dose (±10 min); and 24 hours post-dose (+1 hour);
Cycle 2, 3, 4, and 6 Day 1: pre-dose only.

On a condition that patient requires a dose change, additional PK samples to ensure safety and efficacy parameters, collected at the first cycle following the dosing change, at the same time points as Cycle 1 Day 1 and Cycle 1 Day 15, may be required. Subsequent pre-dose PK samples may be requested at the next 3 cycles following the dosing change.

k. Response Evaluations and Measurements

Response and progression will be evaluated in this study using RECIST Version 1.1. Lesions are either measurable or non-measurable according to the criteria. The actual number of dose levels to be explored in this study depended on determination of the non-tolerable dose based on DLTs. Other safety data, as well as PK profiles observed during the conduct of the study and any trends for anti-tumor activity were collected. Treatment cycles occurred consecutively. Evaluation of a cohort of at least 3 patients completing 1 cycle of treatment (28 days) is required prior to proceeding to the next dose level. In addition, selected dose levels may be expanded to approximately 20 patients (per dose level).

The following analysis populations will be used:
full Analysis Set (FAS)/Safety Analysis Set (SAS) is defined as all patients who have received at least one dose of study treatment. Patients will be included in the cohort in which they have been actually treated;
Efficacy Evaluable Set (EFF) is defined as all patients who have received any dose of study treatment and have at least one adequate post-baseline response assessment; and
PK Analysis Set (PAS) is defined as all patients who have received at least one dose of study treatment and have at least one sample collection of blood with a measurable concentration of study drug in plasma.

Demographic and baseline disease characteristics were summarized descriptively. Data included demographic features such as age, sex, and race, as well as disease-specific characteristics. The number and percentages of patients screened, randomized, treated, completed the treatment/study, and withdrawn from treatment/study for any reasons was presented overall and by dose level.

All efficacy analyses were performed using the Efficacy Evaluable Analysis Set. Objective Response Rate (ORR), defined as the proportion of patients who have measurable disease at baseline with confirmed response (CR) or partial response (PR) (i.e., 2 CRs or PRs at least 4 weeks apart) according to the RECIST Version 1.1 criteria. Clinical Benefit Rate (CBR), defined as the proportion of patients with CR, PR, or stable disease (SD) (where SD is ≥24 weeks) according to the RECIST Version 1.1 criteria. Duration of Response (DOR), defined as the time between first documentation of a response (CR or PR) and first evidence of PD according to RECIST v1.1 or death due to any cause. Disease Control Rate (DCR), defined as the proportion of patients with a best overall response of CR, PR, or SD. Progression Free Survival (PFS), defined as the time from the first day of study drug administration (Day 1) until disease progression as defined by the RECIST Version 1.1 criteria, or death on study, whichever occurs first. Patients who are alive and free from disease progression were censored at the date of last tumor assessment. For ORR and CBR, estimates and the associated 95% CIs (based on the Clopper-Pearson method), at each dose level were calculated. The absolute and relative difference in ORR and CBR between the two treatment groups was presented. For PFS, Kaplan-Meier curves were generated and the median time to event, and the associated 95% CIs were provided. The hazard ratio and the 95% CIs for these endpoints between the two treatment groups was calculated.

### 1. Definition of Measurable and Non-Measurable Tumor Lesions

Patients with at least one tumor lesion or malignant lymph node (either measurable and/or non-measurable) that can be accurately assessed at baseline can be included in the study. At baseline, tumor lesions/lymph nodes will be categorized as measurable or non-measurable as follows:

### Measurable:

Tumor lesions: To be considered measurable disease, tumor lesions must be accurately measured in at least one dimension (longest diameter in the plane of measurement is to be recorded) with a minimum size of 10 mm by computed tomography (CT scan slice thickness/interval no greater than 5 mm).

Malignant lymph nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

### Non-Measurable:

All other lesions, including small lesions (longest diameter <10 mm or pathological lymph nodes with ≥10 to <15 mm short axis at baseline). Truly non-measurable lesions include the following: leptomeningeal disease, ascites, pleural/pericardial effusion, inflammatory breast disease, lymphangitic involvement of skin or lung, and abdominal masses/abdominal organomegaly identified by physical examination that is not measurable by CT or MRI. Previously irradiated lesions or lesions subjected to other local-regional therapy. Note: These lesions may be considered measurable disease if there has been demonstrated progression.

m. Special Consideration Regarding Lesion Measurability

### Bone Lesions

Bone scan, PET scan, or plain films are not considered adequate imaging techniques to measure bone lesions. However, these techniques can be used to confirm the presence or disappearance of bone lesions. Lytic bone lesions or mixed lytic-blastic lesions, with identifiable soft tissue components, can be considered measurable if the soft tissue component meets the definition of measurability described above. Blastic bone lesions are considered non-measurable.

### Cystic Lesions

Cystic lesions thought to represent cystic metastases can be considered measurable lesions if they meet the criteria for measurability from a radiological point of view, but if non-cystic lesions are present in the same patient, these should be selected as target lesions.

n. Definition of Target and Non-Target Lesions

### Target Lesions:

A maximum of 5 measurable lesions (with a maximum of 2 lesions per organ), representative of all involved organs should be identified as target lesions at baseline. Pathological lymph nodes which are defined as measurable and may be identified as target lesions must meet the criterion of a short axis of ≥15 mm by CT scan. Target lesions should be selected on the basis of their size (longest diameter for non-nodal lesions or short axis for nodal lesions), but in addition should be those that lend themselves to reproducible repeated measurements. It may be the case that, on occasion, the largest lesion does not lend itself to reproducible measurement in which circumstance the next largest lesion, which can be measured reproducibly, should be selected. A sum of the diameters (longest for non-nodal lesions, short axis for nodal lesions) for all target lesions will be calculated and reported as the baseline sum of diameters. If lymph nodes are selected as measurable lesions, only the short axis is added into the sum, even if the nodes regress to below 10 mm in the study. The baseline sum of diameters will be used as reference to further characterize any objective tumor regression with regards to measurable disease.

### Special Cases:

If a target lesion has completely disappeared, the longest diameter should be recorded as 0 mm. If a target lesion is believed to be present and is faintly seen but too small to measure, a default value of 5 mm should be assigned. If an accurate measure can be given, this should be recorded, even if it is below 5 mm. When nodal disease is included in the sum of target lesions and the nodes decrease to ‘normal’ size (<10 mm), they may still have a measurement reported on scans. This measurement should be recorded even though the nodes are normal in order to not overstate progression should it be based on increase in size of the nodes. If a target lesion splits into two or more parts, then record the sum of the diameters of those parts. If two or more target lesions merge, then the sum of the diameters of the combined lesion should be recorded for one of the lesions and 0 mm recorded for the other lesion(s). If a target lesion cannot be measured accurately due to it being too large, provide an estimate of the size of the lesion.

### Non-Target Lesions:

All other lesions (or sites of disease) including pathological lymph nodes (those with short axis ≥10 mm but <15 mm) should be identified as non-target lesions (NTLs) and should also be recorded at baseline. Nodes that have a short axis <10 mm are considered non-pathological and should not be recorded or followed. In addition, it is possible to record multiple NTLs involving the same organ as a single item on the case record form (e.g., ‘multiple enlarged pelvic lymph nodes’ or ‘multiple liver metastases’).

o. Methods of Assessment

The same method of assessment and the same technique should be used to characterize each identified and recorded lesion at baseline and during follow-up visits. All baseline evaluations should be performed as close as possible to the treatment start and never more than 4 weeks before the beginning of the treatment.

### CT, MRI:

CT scanning with IV contrast is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. If IV contrast cannot be administered (for example, in the situation of allergy to contrast), a non-contrast CT of the chest is still preferred over MRI or chest X-ray. MRI is also acceptable and can be used when CT is not feasible or is medically contraindicated.

### FDG-PET:

FDG-PET scans may be used as a method for identifying new lesions in the assessment of progression, according with the following algorithm: New lesions will be recorded where there is positive FDG uptake (defined as when an uptake greater than twice that of the surrounding tissue is observed) not present on baseline FDG-PET scan or in a location corresponding to a new lesion by CT/MRI at the same visit. If there is no baseline FDG-PET scan available, and no evidence of new lesions by CT/MRI then follow-up CT/MRI assessments should be continued, scheduled as per protocol or clinically indicated, in order to confirm new lesions.

p. Tumor Response Evaluation

The definitions of the criteria used to determine objective tumor response are provided in Table 3.

TABLE 3
Evaluation criteria of target lesion
Evaluation of target lesion

| | |
|---|---|
| Complete | Disappearance of all target lesions since baseline. Any pathological lymph |
| Response | nodes must have a reduction in short axis to <10 mm. |
| (CR) | |
| Partial | At least a 30% decrease in the sum of the diameters of target lesions, taking |
| Response | as reference the baseline sum of diameters. |
| (PR) | |
| Stable Disease | Neither sufficient shrinkage to qualify for PR nor sufficient increase to |
| (SD) | qualify for PD. Note, for the purpose of this protocol, SD is defined by at |
| | least a 4-week interval. |
| Progressive | At least a 20% increase in the sum of diameters of target lesions, taking as |
| Disease (PD) | reference the smallest sum on study or nadir (this includes the baseline sum |
| | if that is the smallest on study). In addition to the relative increase of 20%, |
| | the sum must also demonstrate an absolute increase of at least 5 mm. |
| Not Evaluable | Only relevant if any of the target lesions were not assessed or not evaluable. |
| (NE) | Note: If the sum of diameters of assessed lesions meets the progressive |
| | disease criteria, progressive disease overrides not evaluable as a target |
| | lesion response. |

Evaluation of non-target lesions

| | |
|---|---|
| Complete | Disappearance of all non-target lesions since baseline. All lymph nodes |
| Response | must be non-pathological in size (<10 mm short axis). |
| (CR) | |
| Non-CR/Non- | Persistence of one or more non-target lesion(s) and/or maintenance of |
| PD | tumor marker level above normal limits and no lesions considered to have |
| | unequivocal progression (PD). |
| Progression | Unequivocal progression of existing non-target lesions indicative of a |
| (PD) | substantial worsening in non-target disease. Unequivocal progression may |
| | be due to an important progression in one lesion only or in several lesions. |
| | In all cases the progression MUST be clinically significant for the |
| | physician to consider changing (or stopping) therapy. |
| Not Evaluable | Only relevant when one or some of the non-target lesions were not assessed |
| (NE) | and, in the Investigator's opinion, they are not able to provide an evaluable |
| | overall non-target lesion assessment at this visit. |
| | Note: For patients without target lesions at baseline, this is relevant if any |
| | of the non-target lesions were not assessed at this visit and the progression |
| | criteria have not been met. |

To achieve ‘unequivocal progression’ on the basis of non-target lesions, there must be an overall level of substantial worsening in non-target disease such that, even in presence of SD, PR or CR in target lesions, the overall tumor burden has increased sufficiently to merit discontinuation of therapy. A modest ‘increase’ in the size of one or more non-target lesions is usually not sufficient to qualify for unequivocal progression status.

### New Lesions:

The presence of one or more new lesions is assessed as disease progression. A lesion identified at a follow-up assessment in an anatomical location that was not scanned at baseline is considered a new lesion and will indicate disease progression. The finding of a new lesion should be unequivocal: i.e. not attributable to differences in scanning technique, change in imaging modality or findings thought to represent something other than tumor. If a new lesion is equivocal, for example because of its small size, the treatment and tumor assessments should be continued until the new lesion has been confirmed. If repeat scans confirm there is a new lesion, then the progression date should be declared using the date of the initial scan.

The overall response is evaluated according to the criteria in Table 4.

TABLE 4
Evaluation of overall response in solid tumors

| Target lesions | Non-Target lesions | New Lesions | Overall response |
|---|---|---|---|
| CR | CR | No | CR |
| CR | NA | No | CR |
| NA | CR | No | CR |
| CR | Non-CR/Non-PD | No | PR |
| CR | NE | No | PR |
| PR | Non-PD or NE | No | PR |
| SD | Non-PD or NE | No | SD |
| NA | Non-CR/Non-PD | No | Non-CR/Non-PD |
| NE | Non-PD or NE | No | NE |
| NA | NE | No | NE |
| PD | Any | Yes or No | PD |
| Any | PD | Yes or No | PD |
| Any | Any | Yes | PD |

CR = complete response, PR = partial response, SD = stable disease, PD = progressive disease, NE = not evaluable, NA = not applicable (relevant when no target lesions/non-target lesions at baseline).

q. Special Notes on Response Evaluation

### Missing Assessments and Non-Evaluable Designation:

When no imaging/measurement is done at all at a particular time point, the patient is not evaluable (NE) at that time point. If only a subset of lesion measurements is made at an assessment, usually the case is also considered NE at that time point, unless a convincing argument can be made that the contribution of the individual missing lesion(s) would not change the assigned time point response. This would be most likely to happen in the case of PD.

### Symptomatic Progression:

Patients with a global deterioration of health status requiring discontinuation of treatment without objective evidence of PD at that time should be reported as “symptomatic deterioration.” Every effort should be made to document objective progression even after discontinuation of treatment. Symptomatic deterioration is not a descriptor of an objective response: it is a reason for stopping study treatment.

### Confirmation of Response:

Confirmation of response (by repeat scans after a minimum of 4 weeks) is required for studies in which response rate is the primary endpoint but is not required in randomized studies or studies with primary survival endpoints (i.e., where response is not a primary endpoint).

Safety was assessed through the analysis of the reported incidence of treatment-emergent AEs. Treatment-emergent AEs are those with an onset on or after the initiation of therapy and will be graded according to NCI CTCAE Version 5.0. Other safety endpoints, including laboratory results, vital signs, and ECG findings, were summarized for all patients in the Safety set.

r. Pharmacokinetics

Plasma concentrations of compound (1-g) were used to calculate the PK parameters. These parameters were listed by individual patient and summarized by descriptive statistics (means, medians, ranges, standard deviations, and coefficients of variation as appropriate) by cohort.

Compound (1-g) was administered orally to patients once a day at 100, 200, 300 mg, 400 mg, and 600 mg doses.

Initial PK patient data from the phase I clinical trials included patients that were administered compound (1-g) orally once a day at 100, 200, and 300 mg doses. This data is presented in Table 5 below.

Subsequently, additional patients were enrolled in the ongoing clinical study and the compound (1-g) dose levels were further increased to include 400 mg and 600 mg doses. The compound (1-g) dose levels investigated to date include: 100 mg (n=4), 200 mg (n=12), 300 mg (n=11), 400 mg (n=4), and 600 mg (n=4).

TABLE 5
Initial pharmacokinetic results of compound (1-g)

| | | | | | |
|---|---|---|---|---|---|
| Compound | 100 mg | 200 mg | 300 mg | 400 mg | 600 mg |
| (1-g) | (n = 3) | (n = 12) | (n = 10) | (n = 6) | (n = 4) |

| C1D1 | Mean | CV(%) | Mean | CV(%) | Mean | CV(%) | Mean | CV(%) | Mean | CV(%) |
|---|---|---|---|---|---|---|---|---|---|---|
| T½ (hour) | 20.18 | 21.38 | 38.27 | 87.36 | 31.19 | 37.07 | 30.39 | 40.84 | 40.91 | 62.97 |
| Tmax*(hour) | 12.67 | 77.89 | 5.50 | 27.41 | 5.80 | 30.19 | 6.00 | 29.81 | 4.50 | 55.92 |
| Cmax(ng/mL) | 432.00 | 55.38 | 629.83 | 35.91 | 963.20 | 33.58 | 1065.83 | 48.78 | 1392.50 | 5.76 |
| AUC_{0-24 h} | 7573.52 | 53.89 | 11226.23 | 31.66 | 16614.17 | 39.33 | 18839.83 | 45.46 | 26059.78 | 6.74 |
| (ng*hr/mL) | | | | | | | | | | |

*Median value for T max

| Compound | 100 mg | | 200 mg | | 300 mg | | 400 mg | | 600 mg | |
|---|---|---|---|---|---|---|---|---|---|---|
| (1-g) | (n = 3) | | (n = 12) | | (n = 10) | | (n = 5) | | (n = 4) | |
| C1D15 | Mean | CV(%) | Mean | CV(%) | Mean | CV(%) | Mean | CV(%) | Mean | CV(%) |
| T½ (hour) | 32.08 | 30.22 | 48.76 | 84.59 | 32.90 | 29.24 | 44.88 | 66.73 | 27.55 | 10.76 |
| Tmax*(hour) | 6.67 | 17.32 | 4.00 | 47.67 | 4.80 | 21.52 | 4.40 | 20.33 | 6.00 | 27.22 |
| Cmax(ng/mL) | 1008.67 | 46.93 | 1341.75 | 32.22 | 2186.00 | 33.66 | 2362.00 | 24.43 | 2682.50 | 29.06 |
| AUC_{0-24 h} | 19522.33 | 44.68 | 26016.83 | 29.35 | 41777.95 | 38.91 | 45888.50 | 24.28 | 48580.00 | 23.77 |
| (ng*hr/mL) | | | | | | | | | | |

*Median value for T max

| Compound | 100 mg | | 200 mg | | 300 mg | | 400 mg | | 600 mg | |
|---|---|---|---|---|---|---|---|---|---|---|
| (1-h) | (n = 1) | | (n = 12) | | (n = 10) | | (n = 6) | | (n = 4) | |
| C1D1 | Mean | CV(%) | Mean | CV(%) | Mean | CV(%) | Mean | CV(%) | Mean | CV(%) |
| T½ ^{#}(hour) | | | | | | | | | | |
| Tmax*(hour) | 24.00 | NA | 24.00 | 0.00 | 24.00 | 0.00 | 24.00 | 0.00 | 24.00 | 0.00 |
| Cmax | 27.80 | NA | 68.78 | 31.71 | 111.54 | 49.92 | 118.17 | 38.09 | 169.75 | 27.14 |
| (ng/mL) | | | | | | | | | | |
| AUC_{0-24 h} | 331.70 | NA | 1025.77 | 31.63 | 1610.82 | 47.81 | 1744.63 | 38.64 | 2470.08 | 16.18 |
| (ng*hr/mL) | | | | | | | | | | |

^{#} T ½ cannot be simulated due to limited elimination phase;
*Median value for T max

| Compound | 100 mg | | 200 mg | | 300 mg | | 400 mg | | 600 mg | |
|---|---|---|---|---|---|---|---|---|---|---|
| (1-h) | (n = 2) | | (n = 12) | | (n = 10) | | (n = 5) | | (n = 4) | |
| C1D15 | Mean | CV(%) | Mean | CV(%) | Mean | CV(%) | Mean | CV(%) | Mean | CV(%) |
| T½ ^{#}(hour) | | | | | | | | | | |
| Tmax*(hour) | 12.00 | 141.42 | 7.17 | 81.98 | 5.80 | 25.44 | 4.80 | 63.19 | 10.00 | 96.61 |
| Cmax(ng/mL) | 325.50 | 81.46 | 428.50 | 36.57 | 680.20 | 55.95 | 820.00 | 25.37 | 828.50 | 12.62 |
| AUC_{0-24 h} | 7075.75 | 79.35 | 9540.50 | 32.09 | 15442.45 | 57.98 | 17909.60 | 24.30 | 18727.75 | 16.08 |
| (ng*hr/mL) | | | | | | | | | | |

^{#} T ½ cannot be simulated due to limited elimination phase;
*Median value for T max

As shown in Table 5, patients were orally dosed with compound 1-g, plasma PK indicated that compound 1-g was partially metabolized to form compound 1-h, with results showing more significant formation of compound 1-h from C1D15 than from C1D1.

s. Data summary

Preliminary results showed dose-dependent increases in Cₘₐₓ and AUC₀₋₂₄ₕ for compound (1-g). The median Tₘₐₓ ranged from 4 to 13 hours across the dose levels. The mean effective T_{1/2} at steady state ranged from 20.18 to 48.8 hours. Cₘₐₓ values ranged from 432 to 2683 ng/mL. AUC₀₋₂₄ₕ values ranged from 7574 to 48580 ng*hr/mL.

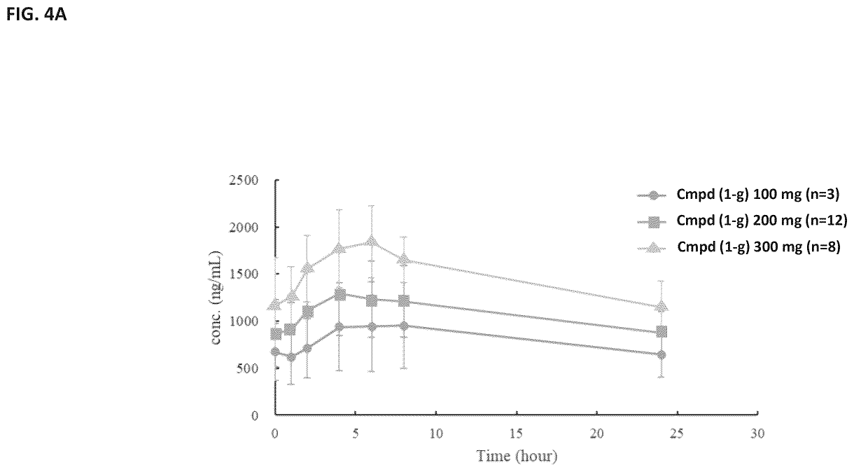

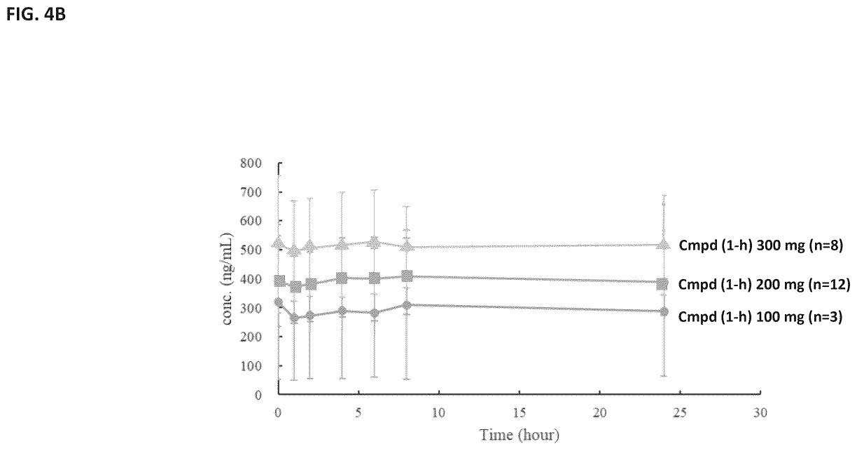

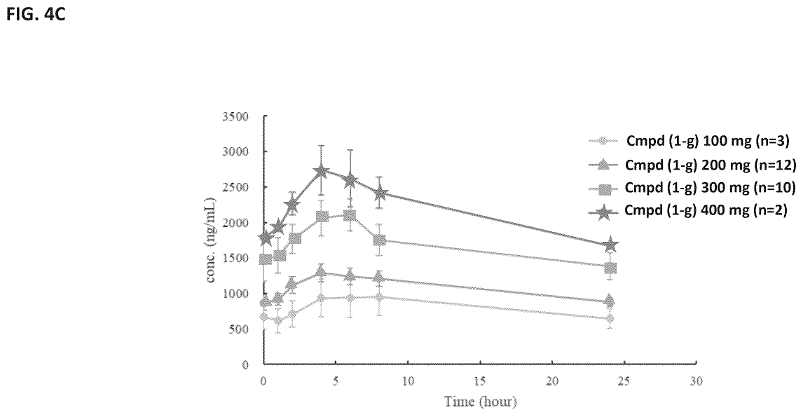

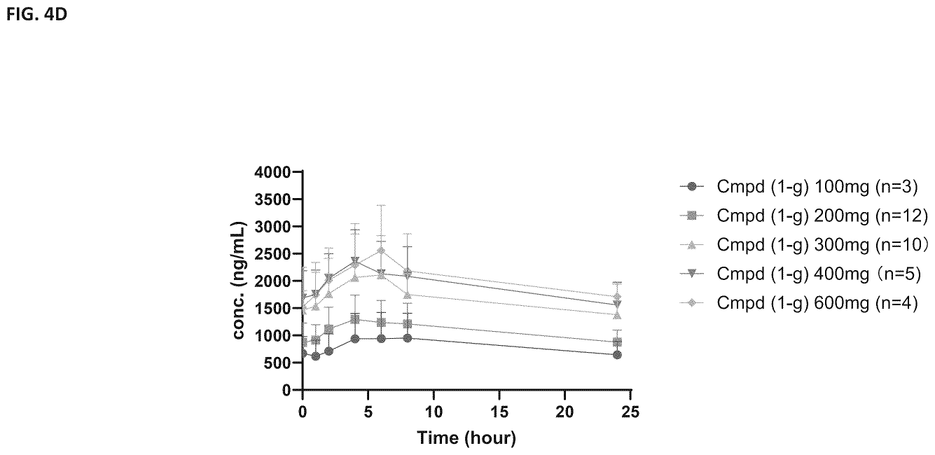

FIG. 4A depicts the plasma concentration of compound (1-g) (ng/mL) at different doses over the course of 24 hours post-dosing on day 15.

FIG. 4B depicts the mean concentration of compound (1-h) (ng/mL) over the course of 24 hours post-dosing on day 15. Compound (1-h) was quantified via HPLC from patient pharmacokinetic samples after patients were treated with compound (1-g). The data showed that compound (1-g) was partially converted to compound (1-h) after patients were treated with compound (1-g).

FIG. 4C depicts the plasma concentration of compound (1-g) (ng/mL) over the course of 24 hours post-dosing on day 15 for cohort 1 (100 mg), cohort 2 (200 mg), cohort 3 (300 mg), and cohort 4 (400 mg).

FIG. 4D depicts the plasma concentration of compound (1-g) (ng/ml) over the course of 24 hours post-dosing on day 15 for patients who were orally administered 100 mg, 200 mg, 300 mg, 400 mg, and 600 mg doses of compound (1-g) once a day.

As it can be seen from the PK profile of compound (1-g), the pharmacokinetics showed a dose proportional relationship up to 400 mg but almost plateaued at 400 mg (see FIGS. 4A, 4C, 4D).

Results of the ongoing Phase 1 study A total of 37 patients had received at least one dose of compound (1-g) in 5 dose cohorts (100, 200, 300 mg, 400 mg, and 600 mg) at the time of data cutoff. ESR1 mutation status was retrospectively determined at a central laboratory based on circulating tumor DNA samples from study participants. A variant allele fraction of >1% from circulating tumor DNA testing on Cycle 1 Day 1 was used to define ESR1 mutation positivity (ESR1 mutation status data on file). 12 participants had ESR1 mutations and VAF>1%. 30 (81%) participants. The median age was 60 years. Participants were heavily pretreated, and median lines of prior therapy were 5 (range: 1-10) in any setting and 3 (1-8) in the metastatic setting. Prior treatments included CDK4/6 inhibitor (100%), aromatase inhibitor (92%), fulvestrant (81%), novel oral SERD or covalent antagonist (SERCA) (22%), and ER chimeric degrader (11%). Thirty patients had measurable diseases while 7 had non measurable bone lesions only at baseline.

The summary of demography and baseline characteristics safety analysis set for 37 patients is presented in Tables 6 and 7.

TABLE 6
Summary of demography and baseline characteristics safety analysis set

| | Cohort 1 | Cohort 2 | Cohort 3 | Cohort 4 | Cohort 5 | Overall |
|---|---|---|---|---|---|---|
| | 100 mg | 200 mg | 300 mg | 400 mg | 600 mg | Total |
| | (N = 4) | (N = 12) | (N = 11) | (N = 6) | (N = 4) | (N = 37) |
| Age (years) at Informed | | | | | | |
| Consent [a] | | | | | | |
| n | 4 | 12 | 11 | 6 | 4 | 37 |
| Mean | 75.5 | 52.8 | 65.1 | 61.3 | 61.3 | 61.2 |
| Standard Deviation | 5.45 | 9.63 | 11.69 | 12.24 | 13.30 | 12.47 |
| Median | 77.0 | 50.5 | 63.0 | 62.0 | 63.0 | 60.0 |
| Minimum | 68 | 41 | 42 | 44 | 45 | 41 |
| Maximum | 80 | 74 | 81 | 78 | 74 | 81 |
| Age Category (years) | | | | | | |
| [a] - n (%) | | | | | | |
| ≥18-<50 | 0 | 6 (50.0) | 1 (9.1) | 1 (16.7) | 1 (25.0) | 9 (24.3) |
| ≥50-<65 | 0 | 5 (41.7) | 5 (45.5) | 3 (50.0) | 1 (25.0) | 14 (37.8) |
| ≥65 | 4 (100) | 1 (8.3) | 5 (45.5) | 2 (33.3) | 2 (50.0) | 14 (37.8) |
| Sex - n (%) | | | | | | |
| Female | 4 (100) | 12 (100) | 11 (100) | 5 (83.3) | 4 (100) | 36 (97.3) |
| Male | 0 | 0 | 0 | 1 (16.7) | 0 | 1 (2.7) |
| Race - n (%) | | | | | | |
| Black or African | 0 | 3 (25.0) | 0 | 2 (33.3) | 0 | 5 (13.5) |
| American | | | | | | |
| White | 4 (100) | 8 (66.7) | 10 (90.9) | 4 (66.7) | 3 (75.0) | 29 (78.4) |
| Multiple | 0 | 0 | 1 (9.1) | 0 | 0 | 1 (2.7) |
| Not Reported | 0 | 1 (8.3) | 0 | 0 | 1 (25.0) | 2 (5.4) |
| Ethnicity - n (%) | | | | | | |
| Not Hispanic or Latino | 4 (100) | 10 (83.3) | 9 (81.8) | 6 (100) | 3 (75.0) | 32 (86.5) |
| Unknown | 0 | 1 (8.3) | 0 | 0 | 0 | 1 (2.7) |
| Not Reported | 0 | 1 (8.3) | 2 (18.2) | 0 | 1 (25.0) | 4 (10.8) |
| Baseline Weight (kg) | | | | | | |
| n | 4 | 12 | 11 | 6 | 4 | 37 |
| Mean | 78.5 | 81.3 | 73.0 | 76.2 | 69.3 | 76.4 |
| Standard Deviation | 22.48 | 18.22 | 15.11 | 14.98 | 20.34 | 17.03 |
| Median | 76.6 | 76.0 | 66.0 | 74.9 | 65.5 | 74.6 |
| Minimum | 57 | 55 | 57 | 55 | 51 | 51 |
| Maximum | 104 | 116 | 107 | 94 | 95 | 116 |
| Baseline ECOG PS | | | | | | |
| [b] - n (%) | | | | | | |
| 0 | 2 (50.0) | 3 (25.0) | 3 (27.3) | 4 (66.7) | 3 (75.0) | 15 (40.5) |
| 1 | 2 (50.0) | 9 (75.0) | 8 (72.7) | 2 (33.3) | 1 (25.0) | 22 (59.5) |

ECOG PS = Eastern Cooperative Oncology Group Performance Status.
[a] Age is an integer difference in years between date of informed consent and date of birth.
[b] 0—Normal activity; 1—Symptoms, but ambulatory; 2—In bed <50% of the time; 3—In bed >50% of the time; 4—100% bedridden.
Baseline value is defined as the last non-missing value prior to the first dose of Compound (1-g).
Percentage is calculated from number of patients in the column header.

TABLE 7
Summary of demography and baseline characteristics safety analysis
set across the overall total of participants under study (N = 37)

| | |
|---|---|
| Participant characteristics | N = 37 |

| | | |
|---|---|---|
| Median age, year (range) | 60 | (41-81) |

ECOG status at baseline, n (%)

| | | |
|---|---|---|
| 0 | 15 | (41%) |
| 1 | 22 | (59%) |

ESR1 ctDNA baseline status, n (%)

| | | |
|---|---|---|
| Wildtype | 24 | (65%) |
| Mutant (VAF > 1%) | 12 | (32%) |
| Pending | 1 | (3%) |
| Measurable disease, n (%) | 30 | (81%) |
| Median prior regimens, n (range) | 5.0 | (1-10) |
| Prior CDK4/6i, n (%) | 37 | (100%) |
| Prior SERD/SERCA/ER chimeric degrader, n (%) | 34 | (92%) |
| Prior fulvestrant | 30 | (81%) |
| Prior novel SERD/SERCA | 8 | (22%) |
| Prior ER chimeric degrader | 4 | (11%) |
| Prior aromatase inhibitor, n (%) | 34 | (92%) |
| Prior chemotherapy in metastatic setting, n (%) | 20 | (54%) |

Thirty seven patients were evaluable for safety. Among the 37 participants treated, 31 discontinued treatments for the following reasons: disease progression (22 participants), physician decision (2 participants), symptomatic deterioration/clinical disease progression (5 participants), death (1 participant) and withdrawal by subject (1 participant). Six participants were still receiving treatment at the time of the data cutoff. Dose levels at 100 mg, 200 mg, 300 mg, 400 mg, and 600 mg QD have been cleared. No dose limiting toxicities were observed with compound (1-g). Backfilling of certain cohorts for additional safety, efficacy and PK data is ongoing.

29 participants (78%) had at least a treatment emergent adverse event (TEAE) after administration of compound (1-g). The most common TEAEs occurred in more than 10% of participants were nausea (18.9%), fatigue (16.2%), neutrophil count decreased (16.2%), dehydration (13.5%), hot flush (13.5%), constipation (10.8%) (Table 9). The incidence of the TEAEs did not seem to correlate with increasing dose levels. Ten participants (27.0%) experienced Grade 23 TEAEs. There was 1 Grade 5 event of seizure. No increase in Grade ≥3 TEAEs was observed with higher dose. Nine SAEs were experienced by 8 patients. No compound (1-g)-related SAEs were observed. Three deaths were reported. All 3 deaths were assessed as unrelated to compound (1-g). One death was due to an AE (seizure, grade 5), and two other deaths were due to underlying disease occurred during the study. There have been no dose reductions or treatment discontinuations due to TEAEs. Nine participants experienced TEAEs that led to dose interruption. Among them, 3 were in Cohort 1 (100 mg QD), 4 in Cohort 2 (200 mg QD), and 2 in Cohort 3 (300 mg QD). None of these were considered related to compound (1-g). No participant experienced TEAEs that led to dose interruption in Cohort 4 and 5, which could be attributed to smaller sample size and less time on treatment. The listing of TEAEs leading to dose interruption of compound (1-g) is presented in Table 8.

TABLE 8
Treatment-emergent adverse events (TEAEs) leading to dose interruption
or dose modifications of compound (1-g) safety analysis set (N = 37)

| | Cohort/Dose | | | | | |
|---|---|---|---|---|---|---|
| | | | | CTCAE | Related to | |
| | | Duration | | Grade | Compound | Action Taken with |
| Patient ID | SOC/PT/VT | (days) | Outcome/SAE | [b] | (1-g) | Compound (1-g) |

Cohort 1/100 mg QD

| | | | | | | |
|---|---|---|---|---|---|---|
| 1 | SOC: Injury, poisoning | 1 | RECOVERED/ | 2 | No | DRUG INTERRUPTED |
| | and procedural | | RESOLVED/ | | | |
| | complications/ | | No | | | |
| | PT: Fall/ | | | | | |
| | VT: FALL | | | | | |
| 2 | SOC: General disorders | — | RECOVERING/ | 3 | No | DRUG INTERRUPTED |
| | and administration site | | RESOLVING/ | | | |
| | conditions/ | | Yes | | | |
| | PT: Fatigue/ | | | | | |
| | VT: FATIGUE | | | | | |
| 3 | SOC: Psychiatric | 2 | RECOVERED/ | 2 | No | DRUG INTERRUPTED |
| | disorders/ | | RESOLVED/ | | | |
| | PT: Confusional state/ | | No | | | |
| | VT: CONFUSION | | | | | |

Cohort 2/200 mg QD

| | | | | | | |
|---|---|---|---|---|---|---|
| 1 | SOC: Blood and | 28 | RECOVERED/ | 2 | No | DRUG INTERRUPTED |
| | lymphatic system | | RESOLVED/ | | | |
| | disorders/ | | No | | | |
| | PT: Anaemia/ | | | | | |
| | VT: ANEMIA | | | | | |
| | SOC: Blood and | — | NOT | 3 | No | DRUG INTERRUPTED |
| | lymphatic system | | RECOVERED/ | | | |
| | disorders/ | | NOT | | | |
| | PT: Anaemia/ | | RESOLVED/ | | | |
| | VT: ANEMIA | | No | | | |
| 2 | SOC: General disorders | — | NOT | 2 | No | DRUG INTERRUPTED |
| | and administration site | | RECOVERED/ | | | |
| | conditions/ | | NOT | | | |
| | PT: Fatigue/ | | RESOLVED/ | | | |
| | VT: FATIGUE | | No | | | |
| | SOC: Musculoskeletal | — | NOT | 2 | No | DRUG INTERRUPTED |
| | and connective tissue | | RECOVERED/ | | | |
| | disorders/ | | NOT | | | |
| | PT: Muscular | | RESOLVED/ | | | |
| | weakness/ | | No | | | |
| | VT: GENERALIZED | | | | | |
| | MUSCLE | | | | | |
| | WEAKNESS | | | | | |
| 3 | SOC: Nervous system | 20 | RECOVERED/ | 3 | No | DRUG INTERRUPTED |
| | disorders/ | | RESOLVED/ | | | |
| | PT: Seizure/ | | Yes | | | |
| | VT: SEIZURE | | | | | |
| | SOC: Infections and | 20 | RECOVERED/ | 3 | No | DRUG INTERRUPTED |
| | infestations/ | | RESOLVED/ | | | |
| | PT: Sepsis/ | | No | | | |
| | VT: SEPSIS | | | | | |
| 4 | SOC: Musculoskeletal | 56 | RECOVERED/ | 1 | No | DRUG INTERRUPTED |
| | and connective tissue | | RESOLVED/ | | | |
| | disorders/ | | No | | | |
| | PT: Pain in extremity/ | | | | | |
| | VT: PAIN IN | | | | | |
| | EXTREMITY- | | | | | |
| | RIGHT ARM AND | | | | | |
| | RIGHT HAND | | | | | |
| | SOC: Musculoskeletal | — | NOT | 2 | No | DRUG INTERRUPTED |
| | and connective tissue | | RECOVERED/ | | | |
| | disorders/ | | NOT | | | |
| | PT: Pain in extremity/ | | RESOLVED/ | | | |
| | VT: PAIN IN | | No | | | |
| | EXTREMITY- | | | | | |
| | RIGHT ARM AND | | | | | |
| | RIGHT HAND | | | | | |

Cohort 3/300 mg QD

| | | | | | | |
|---|---|---|---|---|---|---|
| 1 | SOC: Blood and | — | NOT | 3 | No | DRUG INTERRUPTED |
| | lymphatic system | | RECOVERED/ | | | |
| | disorders/ | | NOT | | | |
| | PT: Anaemia/ | | RESOLVED/ | | | |
| | VT: ANEMIA | | No | | | |
| | SOC: Renal and | 6 | RECOVERED/ | 3 | No | DRUG INTERRUPTED |
| | urinary disorders/ | | RESOLVED/ | | | |
| | PT: Acute kidney | | Yes | | | |
| | injury/ | | | | | |
| | VT: ACUTE KIDNEY | | | | | |
| | INJURY | | | | | |
| | SOC: Renal and | 7 | RECOVERED/ | 3 | No | DRUG INTERRUPTED |
| | urinary disorders/ | | RESOLVED/ | | | |
| | PT: Acute kidney | | Yes | | | |
| | injury/ | | | | | |
| | VT: ACUTE KIDNEY | | | | | |
| | INJURY | | | | | |
| 2 | SOC: Infections and | 8 | RECOVERED/ | 2 | No | DRUG INTERRUPTED |
| | infestations/ | | RESOLVED/ | | | |
| | PT: Upper respiratory | | No | | | |
| | tract infection/ | | | | | |
| | VT: UPPER | | | | | |
| | RESPIRATORY | | | | | |
| | INFECTION | | | | | |
| | SOC: Musculoskeletal | — | NOT | 2 | No | DRUG INTERRUPTED |
| | and connective tissue | | RECOVERED/ | | | |
| | disorders/ | | NOT | | | |
| | PT: Muscular | | RESOLVED/ | | | |
| | weakness/ | | No | | | |
| | VT: GENERALIZED | | | | | |
| | MUSCLE | | | | | |
| | WEAKNESS | | | | | |

SOC: System Organ Class
PT: Preferred Term
VT: Verbatim Term
SAE = Serious Adverse Event; CTCAE = Common Terminology Criteria for Adverse Events.
[a] MedDRA version: 26.0.
[b] CTCAE version: 5.0.
A TEAE is defined as an AE with an onset on or worsening grade on or after the start of the first dose of study treatment up to 30 days after the end of treatment or up to the start of other anti-cancer therapy (which occurs earlier).
Study Day is calculated as Study Date − Date of First Compound (1-g) Dose + 1 if date is on or after initiation of Compound (1-g), otherwise it is calculated as Study Date − Date of First Compound (1-g) Dose.

TABLE 9
Treatment-emergent adverse events in ≥10% of patients (N = 37)

| | TEAE | | Related-TEAE | | |
|---|---|---|---|---|---|
| | All Grades | Grade ≥ 3 | All Grades | | Grade ≥ 3 |
| | n (%) | n (%) | n (%) | | n (%) |
| Nausea | 7 (19) | 0 | 5 | (14) | 0 |
| Fatigue | 6 (16) | 1 (3) | 2 | (5) | 0 |
| Neutrophil | 6 (16) | 1 (3) | 4 | (11) | 0 |
| count decreased | | | | | |
| Dehydration | 5 (14) | 2 (5) | 0 | | 0 |
| Hot flush | 5 (14) | 0 | 5 | (14) | 0 |
| Constipation | 4 (11) | 0 | 3 | (8) | 0 |

All TEAEs are also listed by Cohorts in Table 10.

TABLE 10
TEAEs by preferred term safety evaluation analysis

| | Cohort 1 | Cohort 2 | Cohort 3 | Cohort 4 | Cohort 5 | |
|---|---|---|---|---|---|---|
| | (100 mg QD) | (200 mg QD) | (300 mg QD) | (400 mg QD) | (600 mg QD) | Overall |
| Preferred Term | (N = 4) | (N = 12) | (N = 11) | (N = 6) | (N = 4) | (N = 37) |
| [a] | n (%) | n (%) | n (%) | n (%) | n (%) | n (%) |
| Patients with | 4 (100) | 11 (91.7) | 8 (72.7) | 4 (66.7) | 2 (50.0) | 29 (78.4) |
| Any TEAE | | | | | | |
| Nausea | 1 (25.0) | 1 (8.3) | 4 (36.4) | 1 (16.7) | 0 | 7 (18.9) |
| Fatigue | 1 (25.0) | 2 (16.7) | 1 (9.1) | 2 (33.3) | 0 | 6 (16.2) |
| Neutrophil Count | 1 (25.0) | 1 (8.3) | 1 (9.1) | 2 (33.3) | 1 (25.0) | 6 (16.2) |
| Decreased | | | | | | |
| Dehydration | 3 (75.0) | 1 (8.3) | 1 (9.1) | 0 | 0 | 5 (13.5) |
| Hot Flush | 1 (25.0) | 1 (8.3) | 2 (18.2) | 0 | 1 (25.0) | 5 (13.5) |
| Constipation | 0 | 1 (8.3) | 1 (9.1) | 1 (16.7) | 1 (25.0) | 4 (10.8) |
| Dyspnoea | 0 | 1 (8.3) | 1 (9.1) | 1 (16.7) | 0 | 3 (8.1) |
| Insomnia | 1 (25.0) | 1 (8.3) | 0 | 1 (16.7) | 0 | 3 (8.1) |
| Upper Respiratory | 1 (25.0) | 1 (8.3) | 1 (9.1) | 0 | 0 | 3 (8.1) |
| Tract Infection | | | | | | |
| Back Pain | 0 | 1 (8.3) | 1 (9.1) | 0 | 1 (25.0) | 3 (8.1) |
| Diarrhoea | 1 (25.0) | 0 | 1 (9.1) | 1 (16.7) | 0 | 3 (8.1) |
| Hyperhidrosis | 1 (25.0) | 1 (8.3) | 0 | 0 | 1 (25.0) | 3 (8.1) |
| Muscular | 0 | 1 (8.3) | 0 | 1 (16.7) | 0 | 2 (5.4) |
| Weakness | | | | | | |
| Platelet Count | 0 | 0 | 1 (9.1) | 1 (16.7) | 0 | 2 (5.4) |
| Decreased | | | | | | |
| Pneumonia | 1 (25.0) | 1 (8.3) | 0 | 0 | 0 | 2 (5.4) |
| Rash Maculo- | 1 (25.0) | 1 (8.3) | 0 | 0 | 0 | 2 (5.4) |
| Papular | | | | | | |
| Tremor | 1 (25.0) | 0 | 1 (9.1) | 0 | 0 | 2 (5.4) |
| Urinary Tract | 0 | 1 (8.3) | 1 (9.1) | 0 | 0 | 2 (5.4) |
| Infection | | | | | | |
| Vomiting | 0 | 1 (8.3) | 1 (9.1) | 0 | 0 | 2 (5.4) |
| Abdominal Pain | 1 (25.0) | 1 (8.3) | 0 | 0 | 0 | 2 (5.4) |
| Anaemia | 0 | 1 (8.3) | 1 (9.1) | 0 | 0 | 2 (5.4) |
| Arthralgia | 0 | 2 (16.7) | 0 | 0 | 0 | 2 (5.4) |
| Dysuria | 0 | 1 (8.3) | 1 (9.1) | 0 | 0 | 2 (5.4) |
| Fall | 1 (25.0) | 1 (8.3) | 0 | 0 | 0 | 2 (5.4) |
| Flatulence | 0 | 0 | 1 (9.1) | 1 (16.7) | 0 | 2 (5.4) |
| Hypokalaemia | 0 | 1 (8.3) | 1 (9.1) | 0 | 0 | 2 (5.4) |
| Oedema | 0 | 0 | 1 (9.1) | 1 (16.7) | 0 | 2 (5.4) |
| Peripheral | | | | | | |
| Acute Kidney | 0 | 0 | 1 (9.1) | 0 | 0 | 1 (2.7) |
| Injury | | | | | | |
| Adrenal | 0 | 1 (8.3) | 0 | 0 | 0 | 1 (2.7) |
| Insufficiency | | | | | | |
| Axillary Vein | 0 | 1 (8.3) | 0 | 0 | 0 | 1 (2.7) |
| Thrombosis | | | | | | |
| Basal Cell | 1 (25.0) | 0 | 0 | 0 | 0 | 1 (2.7) |
| Carcinoma | | | | | | |
| Breast Pain | 0 | 1 (8.3) | 0 | 0 | 0 | 1 (2.7) |
| Confusional State | 1 (25.0) | 0 | 0 | 0 | 0 | 1 (2.7) |
| Covid-19 | 1 (25.0) | 0 | 0 | 0 | 0 | 1 (2.7) |
| Pneumonia | | | | | | |
| Dyspepsia | 1 (25.0) | 0 | 0 | 0 | 0 | 1 (2.7) |
| Fracture | 0 | 1 (8.3) | 0 | 0 | 0 | 1 (2.7) |
| Fungal Infection | 0 | 0 | 1 (9.1) | 0 | 0 | 1 (2.7) |
| Lymphocyte | 0 | 1 (8.3) | 0 | 0 | 0 | 1 (2.7) |
| Count Decreased | | | | | | |
| Ophthalmic | 0 | 1 (8.3) | 0 | 0 | 0 | 1 (2.7) |
| Migraine | | | | | | |
| Oral Pain | 0 | 0 | 1 (9.1) | 0 | 0 | 1 (2.7) |
| Pain In Extremity | 0 | 1 (8.3) | 0 | 0 | 0 | 1 (2.7) |
| Pharyngitis | 0 | 1 (8.3) | 0 | 0 | 0 | 1 (2.7) |
| Pleural Effusion | 0 | 0 | 1 (9.1) | 0 | 0 | 1 (2.7) |
| Rhinitis Allergic | 0 | 0 | 1 (9.1) | 0 | 0 | 1 (2.7) |
| Rib Fracture | 1 (25.0) | 0 | 0 | 0 | 0 | 1 (2.7) |
| Seizure | 0 | 1 (8.3) | 0 | 0 | 0 | 1 (2.7) |
| Sepsis | 0 | 1 (8.3) | 0 | 0 | 0 | 1 (2.7) |
| Musculoskeletal | 0 | 1 (8.3) | 0 | 0 | 0 | 1 (2.7) |
| Stiffness | | | | | | |
| Abdominal | 0 | 0 | 1 (9.1) | 0 | 0 | 1 (2.7) |
| Distension | | | | | | |
| Alopecia | 0 | 0 | 0 | 1 (16.7) | 0 | 1 (2.7) |
| Anxiety | 0 | 0 | 1 (9.1) | 0 | 0 | 1 (2.7) |
| Cough | 0 | 1 (8.3) | 0 | 0 | 0 | 1 (2.7) |
| Dizziness | 0 | 0 | 1 (9.1) | 0 | 0 | 1 (2.7) |
| Dysgeusia | 0 | 0 | 0 | 1 (16.7) | 0 | 1 (2.7) |
| Eructation | 0 | 0 | 0 | 1 (16.7) | 0 | 1 (2.7) |
| Haematoma | 0 | 0 | 1 (9.1) | 0 | 0 | 1 (2.7) |
| Hyperglycaemia | 0 | 1 (8.3) | 0 | 0 | 0 | 1 (2.7) |
| Hypotension | 0 | 0 | 0 | 1 (16.7) | 0 | 1 (2.7) |
| Influenza Like | 0 | 1 (8.3) | 0 | 0 | 0 | 1 (2.7) |
| Illness | | | | | | |
| Lower Respiratory | 0 | 1 (8.3) | 0 | 0 | 0 | 1 (2.7) |
| Tract Congestion | | | | | | |
| Muscle Spasm | 0 | 0 | 0 | 1 (16.7) | 0 | 1 (2.7) |
| Nail Disorder | 0 | 0 | 0 | 1 (16.7) | 0 | 1 (2.7) |
| Nasal Congestion | 0 | 1 (8.3) | 0 | 0 | 0 | 1 (2.7) |
| Productive Cough | 0 | 1 (8.3) | 0 | 0 | 0 | 1 (2.7) |
| Vaginal | 0 | 0 | 1 (9.1) | 0 | 0 | 1 (2.7) |
| Haemorrhage | | | | | | |

TEAE = Treatment-Emergent Adverse Event; CTCAE = Common Terminology Criteria for Adverse Events; SOC = System Organ Class; PT = Preferred Term.
[a] MedDRA version: 26.0.
A TEAE is defined as an AE with an onset on or worsening grade on or after the start of the first dose of study treatment up to 30 days after the end of treatment or up to the start of other anti-cancer therapy (which occurs earlier).
PT are sorted by decreasing overall frequency count. Subjects with multiple events in the same preferred term are counted only once in that preferred term. Subjects with events in more than 1 preferred term are counted once in each of those preferred terms.
Percentage is calculated from number of patients in the column header.

Fifteen participants (40.5%) experienced compound (1-g) related TEAEs, the most common of which were nausea (13.5%), hot flush (13.5%), and neutrophil count decrease (10.8%) as shown in Table 12. No dose-related trends were evident in the incidence of the events between 100 mg and 600 mg. All treatment-related AEs were Grade 1 or 2. There is no Grade≥3 TEAEs were considered as related to compound (1-g) (Table 11).

TABLE 11
Compound (1-g) related TEAEs of CTCAE grade >=3 by system
organ class and preferred term safety analysis set

| | | | | | |
|---|---|---|---|---|---|
| Cohort 1 | Cohort 2 | Cohort 3 | Cohort 4 | Cohort 5 | |
| (100 mg | (200 mg | (300 mg | (400 mg | (600 mg | |
| QD) | QD) | QD) | QD) | QD) | Overall |
| (N = 4) | (N = 12) | (N = 11) | (N = 6) | (N = 4) | (N = 37) |
| n (%) | n (%) | n (%) | n (%) | n (%) | n (%) |

| | |
|---|---|
| SOC/PT | No data meets output criteria |

TEAE = Treatment-Emergent Adverse Event; CTCAE = Common Terminology Criteria for Adverse Events; SOC = System Organ Class; PT = Preferred Term.
[a] MedDRA version: 26.0.
A TEAE is defined as an AE with an onset on or worsening grade on or after the start of the first dose of study treatment up to 30 days after the end of treatment or up to the start of other anti-cancer therapy (which occurs earlier).
PT are sorted by decreasing overall frequency count. Subjects with multiple events in the same preferred term are counted only once in that preferred term. Subjects with events in more than 1 preferred term are counted once in each of those preferred terms.
Percentage is calculated from number of patients in the column header.

The summary of compound (1-g) related TEAEs by preferred term safety analysis set is presented in Table 12.

TABLE 12
Compound (1-g) related TEAEs by preferred term safety analysis set

| | Cohort 1 | Cohort 2 | Cohort 3 | Cohort 4 | Cohort 5 | |
|---|---|---|---|---|---|---|
| | (100 mg QD) | (200 mg QD) | (300 mg QD) | (400 mg QD) | (600 mg QD) | Overall |
| Preferred | (N = 4) | (N = 12) | (N = 11) | (N = 6) | (N = 4) | (N = 37) |
| Term [a] | n (%) | n (%) | n (%) | n (%) | n (%) | n (%) |
| Patients with | 2 (50.0) | 3 (25.0) | 5 (45.5) | 3 (50.0) | 2 (50.0) | 15 (40.5) |
| Any Compound | | | | | | |
| (1-g) related | | | | | | |
| TEAE | | | | | | |
| Hot Flush | 1 (25.0) | 1 (8.3) | 2 (18.2) | 0 | 1 (25.0) | 5 (13.5) |
| Nausea | 1 (25.0) | 0 | 3 (27.3) | 1 (16.7) | 0 | 5 (13.5) |
| Neutrophil | 1 (25.0) | 0 | 0 | 2 (33.3) | 1 (25.0) | 4 (10.8) |
| Count | | | | | | |
| Decreased | | | | | | |
| Constipation | 0 | 0 | 1 (9.1) | 1 (16.7) | 1 (25.0) | 3 (8.1) |
| Diarrhea | 1 (25.0) | 0 | 1 (9.1) | 1 (16.7) | 0 | 3 (8.1) |
| Fatigue | 0 | 1 (8.3) | 1 (9.1) | 0 | 0 | 2 (5.4) |
| Alopecia | 0 | 0 | 0 | 1 (16.7) | 0 | 1 (2.7) |
| Arthralgia | 0 | 1 (8.3) | 0 | 0 | 0 | 1 (2.7) |
| Dysgeusia | 0 | 0 | 0 | 1 (16.7) | 0 | 1 (2.7) |
| Eructation | 0 | 0 | 0 | 1 (16.7) | 0 | 1 (2.7) |
| Flatulence | 0 | 0 | 0 | 1 (16.7) | 0 | 1 (2.7) |
| Hyperhidrosis | 0 | 0 | 0 | 0 | 1 (25.0) | 1 (2.7) |
| Nail Disorder | 0 | 0 | 0 | 1 (16.7) | 0 | 1 (2.7) |
| Platelet Count | 0 | 0 | 0 | 1 (16.7) | 0 | 1 (2.7) |
| Decreased | | | | | | |
| Tremor | 1 (25.0) | 0 | 0 | 0 | 0 | 1 (2.7) |
| Vomiting | 0 | 0 | 1 (9.1) | 0 | 0 | 1 (2.7) |

TEAE = Treatment-Emergent Adverse Event; CTCAE = Common Terminology Criteria for Adverse Events; SOC = System Organ Class; PT = Preferred Term.
[a] MedDRA version: 26.0.
A TEAE is defined as an AE with an onset on or worsening grade on or after the start of the first dose of study treatment up to 30 days after the end of treatment or up to the start of other anti-cancer therapy (which occurs earlier).
PT are sorted by decreasing overall frequency count. Subjects with multiple events in the same preferred term are counted only once in that preferred term. Subjects with events in more than 1 preferred term are counted once in each of those preferred terms.
Percentage is calculated from number of patients in the column header.

Twenty-six patients were evaluable for objective efficacy response, defined as having at least 1 measurable lesion at baseline, and an adequate post baseline response assessment as of the data cutoff date. Tumor response was defined using Response Evaluation Criteria in Solid Tumors (RECIST) 1.1 criteria. Four participants (15.4%) achieved confirmed partial response. The summary of objective response rate (ORR) and best objective response is presented in Table 13.

TABLE 13
The summary of objective response rate (ORR) and best objective
response defined by RECIST 1.1 Efficacy Evaluable Analysis Set

| | Cohort 1 | Cohort 2 | Cohort 3 | Cohort 4 | Cohort 5 | |
|---|---|---|---|---|---|---|
| | (100 mg | (200 mg | (300 mg | (400 mg | (600 mg | |
| Response Status | QD) | QD) | QD) | QD) | QD) | Overall |
| Best Objective | (N = 2) | (N = 8) | (N = 9) | (N = 4) | (N = 3) | (N = 26) |
| Response [a] | n (%) | n (%) | n (%) | n (%) | n (%) | n (%) |
| Response - n (%) | 1 (50.0) | 2 (25.0) | 1 (11.1) | 0 | 0 | 4 (15.4) |
| Confirmed | 0 | 0 | 0 | 0 | 0 | 0 |
| Complete | | | | | | |
| Response (cCR) | | | | | | |
| [a] | | | | | | |
| Unconfirmed | 0 | 0 | 0 | 0 | 0 | 0 |
| Complete | | | | | | |
| Response (uCR) | | | | | | |
| Confirmed Partial | 1 (50.0) | 2 (25.0) | 1 (11.1) | 0 | 0 | 4 (15.4) |
| Response (cPR) | | | | | | |
| [a] | | | | | | |
| Unconfirmed | 0 | 0 | 0 | 0 | 0 | 0 |
| Partial Response | | | | | | |
| (uPR) | | | | | | |
| Non-Response - | 1 (50.0) | 6 (75.0) | 8 (88.9) | 4 (100) | 3 (100) | 22 (84.6) |
| n (%) | | | | | | |
| Stable Disease | 1 (50.0) | 3 (37.5) | 4 (44.4) | 2 (50.0) | 3 (100) | 13 (50.0) |
| (SD) [b] | | | | | | |
| Progressive | 0 | 2 (25.0) | 4 (44.4) | 2 (50.0) | 0 | 8 (30.8) |
| Disease (PD) | | | | | | |
| Not Evaluable | 0 | 1 (12.5) | 0 | 0 | 0 | 1 (3.8) |
| (NE) | | | | | | |
| Not Applicable | 0 | 0 | 0 | 0 | 0 | 0 |
| (NA) | | | | | | |
| ORR (CR + PR) - | 1 (50.0) | 2 (25.0) | 1 (11.1) | 0 | NE* | 4 (15.4) |
| n (%) [c] | | | | | | |
| 95% CI [d] | (1.3, 98.7) | (3.2, 65.1) | (0.3, 48.2) | (0.0, 60.2) | (0.0, 70.8) | (4.4, 34.9) |

NE* = Not Estimable
[a] Confirmation by second assessment at least 4 weeks after first assessment.
[b] At least 7 weeks after start of treatment.
[c] ORR is defined as the proportion of patients with a best objective response of CR or PR as defined by RECIST version 1.1 using the Investigator assessment captured on the eCRF.
[d] Two-sided 95% confidence interval (CI) calculated based on the exact Clopper-Pearson for binomial proportions.
Percentage is calculated from number of patients in the column header.
All 4PRs noted above were participants with ESR1 mutation at baseline, resulting in an ORR of 40% (4/10) in ESR1 mutated evaluable participants, as shown in Table 14.

The clinical benefit rate (CBR), as used herein, is defined as confirmed complete response or partial response at any time and stable disease over ≥24 weeks. Evaluable participants need to complete an adequate post-treatment scan. Participants with SD who remain on study for less than 6 months are not included in the denominator for CBR. As shown in Table 14, CBR was 23% (7/30). In the subgroup of CBR evaluable patients (n=10) harboring a confirmed ESR1 mutation, the CBR was 45% (5/11).

The preliminary steady state concentration maximum (Cₘₐₓ) and area under the curve (AUC) for the 100 and 200 mg cohorts were similar (Cₘₐₓ of 1008.7 ng/mL vs. 1341.8 ng/ml respectively; AUC 0-24h of 19,522 ng*hr/mL vs. 26,016.8 ng*hr/mL, respectively), but both were in the expected tolerable and efficacious range based on preclinical data and comparable clinical investigations with another ER chimeric degrader. At 300 mg, the mean Cₘₐₓ was 2186 ng/ml and the AUC 0-24h was 41,778 ng*hr/mL.

The above data show safety, tolerability, and anti-tumor activity for compound (1-g) with no >Grade 3 related AE or dose modifications at doses up to 600 mg orally once daily. Compound (1-g) demonstrated clinical activity with partial responses in 40% of participants with ESR1 mutations. Based on the observed safety, tolerability, PK and preliminary efficacy data, 200 mg and 400 mg doses have been preliminarily chosen for further evaluation in a phase 2 study (see Example 8 below).

### Efficacy

The objective response rate (ORR) and the clinical benefit rate (CBR) were assessed for the participants. The clinical benefit rate (CBR), as used herein, is defined as confirmed complete response or partial response at any time and stable disease over ≥24 weeks. Evaluable participants need to complete an adequate post-treatment scan. Participants with SD who remain on study <6 months are not included in the denominator.

As shown in Table 14, the total ORR was 15% (4/26) in all evaluable participants (n=26), with 4 confirmed PRs. The total CBR which also includes patients with stable disease ≥24 weeks was 23% (7/30). In the subgroup of evaluable patients harboring a confirmed ESR1 mutation, the ORR was 40% (4/10) and the CBR was 45% (5/11).

TABLE 14
Response Assessment

| | Total | ESR1 Mutated |
|---|---|---|
| ORR | 15% (4/26) | 40% (4/10) |
| CBR | 23% (7/30) | 45% (5/11) |

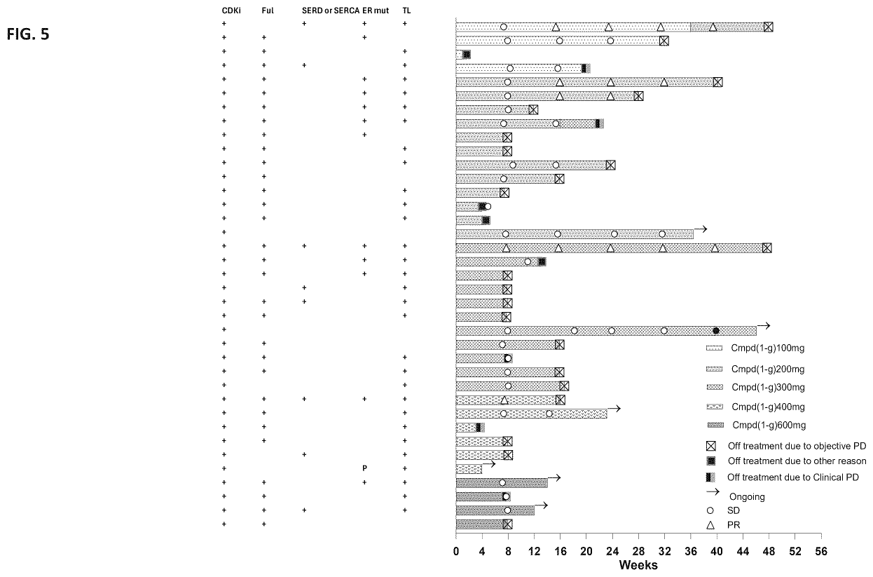

The median treatment time for all treated participants was 84 days (range 12-336), with 6 of 37 participants still receiving compound (1-g). FIG. 5 depicts the treatment duration in weeks for different cohorts of participants that were administered 100 mg, 200 mg, 300 mg, 400 mg, and 600 mg of compound (1-g). The (+) symbol indicates whether participants had previously received prior therapy with CDKi, fulvestrant (FUL), or a SERD or SERCA. A (+) symbol under ER mut indicates that the participant has an ESR1 mutation at baseline (VAF >1%). A (+) symbol under TL indicated that the patient has a measurable target lesion. Each bar represents a single participant, and the length of the bar indicates the time on study for that participant, measured in weeks. Circles indicate that the participant had achieved stable disease and triangles indicate that the participant had achieved a partial response. Arrows indicate that treatment is ongoing.

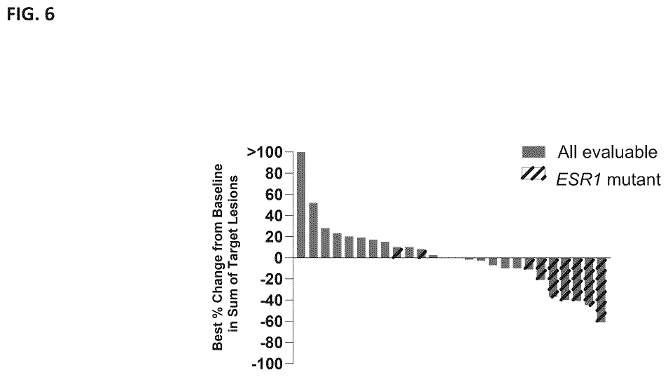

FIG. 6 depicts the change in solid tumor lesions from baseline in evaluable participants (n=26) with ESR1 mutation and wild type (WT) ER. This data shows the best percentage of change in the sum of target lesions from baseline.

The ongoing clinical study data confirms the safety, tolerability, and anti-tumor activity for compound (1-g) with compound (1-g) related AE that required dose modifications at the tested doses when administered orally once daily. Compound (1-g) demonstrated clinical activity, including participants with ESR1 mutations.

### Example 8. a Randomized Phase 2 Study Evaluating Two Dose Levels of Compound (1-g) in Patients with ESR1 Mutated ER+/HER2-Advanced or Metastatic Breast Cancer

In a Phase 2, open-label, randomized study, compound (1-g) is given as a single agent. Approximately 60 participants will be randomized to either 200 mg once daily (QD) or 400 mg QD on a 28-day per cycle schedule. Participants will be stratified based on the presence of measurable disease and time on CDK4/6 inhibitor (<12 months and >12 months). The study will evaluate the anti-tumor activity and safety and tolerability of compound (1-g) at these two dose levels in patients with ESR1 mutated ER+/HER2− advanced or metastatic breast cancer. The main goals of this study are to assess:
Progression-free survival (PFS) at 6 months
Objective response rate (ORR)

TABLE 15
Summary of objectives

| Objectives | Endpoints/Variables: |
|---|---|
| Primary Objectives: | Progression-free survival (PFS) at 6 months |
| Evaluate the anti-tumor activity | Objective response rate (ORR = confirmed complete response |
| | [CR] or partial response [PR]) as assessed by the investigator |
| | per Response Evaluation Criteria in Solid Tumors (RECIST) |
| | Version 1.1 |
| Secondary Objectives: | Incidence and severity of treatment emergent adverse events |
| Evaluate the safety and | (TEAE) and serious adverse events (SAE) |
| tolerability | Incidence of clinically significant Grade 3 or higher laboratory |
| | abnormalities |
| | Frequency of dose modifications |
| Evaluate the clinical benefit | Clinical benefit rate (CBR = CR + PR + stable disease [SD] |
| | for ≥24 weeks) as assessed by the Investigator |
| | Duration of response (DOR) as assessed by the Investigator |
| Evaluate the pharmacokinetic | PK parameters |
| profile | |
| Exploratory Objectives: | Change of ERα expression in tumor tissue before and during |
| Evaluate the pharmacodynamic | therapy if paired biopsies are available |
| effect of Compound (1-g) in | |
| ERα degradation | |
| Evaluate the relationship | Correlation between CTC count before and during therapy and |
| between circulating tumor cell | ORR, CBR, DOR, and PFS |
| (CTC) count and anti-tumor | |
| activity | |
| Evaluate the relationship | Correlation between ctDNA levels before and during therapy |
| between circulating tumor DNA | and ORR, CBR, DOR, and PFS |
| (ctDNA) levels and anti-tumor | |
| activity | |

a. Study Rationale

This phase 2 study is designed to further evaluate two dose levels of compound (1-g) that were evaluated in the phase 1 dose escalation study, through further assessment of efficacy, PK, safety, and tolerability. The rationale for stratification is to balance the number of participants in each cohort who have certain baseline characteristics associated with poorer prognosis. Stratification factors for this trial include presence of target lesions and duration of prior CDK4/6 inhibitor. Among patients with advanced disease treated with fulvestrant, those with measurable visceral metastases had poorer overall survival compared to those without visceral metastases (18 versus 32 months, p=0.029) (see Andrahennadi, et al., Cancers, 2021, 13 (16): 4163). A post-hoc analysis of the EMERALD trial (NCT03778931) showed that, among elacestrant-treated patients with ESR1 mutations, those who had received a prior CDK4/6 inhibitor for less than 12 months in the metastatic setting had poorer PFS compared with those who had received a prior CDK4/6 inhibitor for 12 months or more (4.1 versus 8.6 months) (see Bardia, et al., presented at: San Antonio Breast Cancer Symposium; Dec. 6-10, 2022; San Antonio, TX).

A goal of the randomized phase 2 study is to further evaluate the 200 and 400 mg doses in more participants to better understand the PK profiles at these dose levels and to generate a more thorough exposure-response analysis, while further assessing safety, tolerability, and anti-tumor activity.

b. Patient eligibility

TABLE 16
Eligibility

| | |
|---|---|
| Minimum Age: | 18 Years |
| Maximum Age: | |
| Sex: | All |
| Gender Based: | No |
| Accepts | No |
| Healthy | |
| Volunteers: | |
| Criteria: | Inclusion Criteria: |
| | 1. At least 18 years old at the time of the signature of the informed |
| | consent. |
| | 2. Confirmed diagnosis of ER+/HER2− advanced or metastatic breast |
| | cancer as defined by the American Society of Clinical |
| | Oncologists/College of American Pathologists (ASCO/CAP) |
| | guidelines (Wolff et al. 2018). |
| | 3. Documented ESR1 mutation from a tissue sample or a blood sample. |
| | 4. Received at least 1 line of endocrine therapy including a CDK4/6 inhibitor in the |
| | advanced/metastatic setting. |
| | 5. Presence of at least 1 measurable lesion according to RECIST v1.1 (Appendix |
| | A) or at least 1 predominantly lytic bone lesion. |
| | 6. Eastern Cooperative Oncology Group (ECOG) Performance Status score of 0 or |
| | 1 |
| | 7. Female participants must meet one of the following criteria: |
| | a. Premenopausal or perimenopausal women must be |
| | concurrently given a luteinizing hormone-releasing hormone |
| | (LHRH) agonist starting at least 4 weeks before the start of |
| | study drug and agree to continue the LHRH agonist throughout |
| | the duration of study treatment, have a negative serum |
| | pregnancy test within 7 days of initiating treatment, and agree |
| | to follow guidelines for use of highly effective contraception |
| | during the study and for 90 days following the last dose of |
| | study drug. |
| | b. Be postmenopausal as defined by at least one of the following: |
| | i. Age ≥ 60, or |
| | ii. Spontaneous amenorrhea for ≥12 months following cessation |
| | of all exogenous hormonal treatment, or |
| | iii. 6 months of spontaneous amenorrhea with serum follicle- |
| | stimulating (FSH) and estradiol in the post-menopausal range per |
| | institutional standards, or |
| | iv. Bilateral oophorectomy performed at least 6 weeks before the |
| | start of study drug. |
| | 8. Males with female partners of childbearing potential are required to use two |
| | forms of acceptable contraception, including one barrier method, during their |
| | participation in the study and for 90 days following the last dose. Males must |
| | also refrain from donating sperm during their participation in the study and for |
| | 90 days following the last dose. |
| | 9. Acceptable organ function as evidenced by laboratory data. |
| | a. Renal function, as follows: |
| | i. creatinine clearance of ≥60 mL/min by the Cockcroft-Gault |
| | equation and a normal creatinine per institutional standards |
| | b. Liver function as follows: |
| | i. total bilirubin ≤ 1.5 × ULN or ≤5 × ULN for patients with |
| | Gilberts, and |
| | ii. alanine aminotransferase (ALT) and ≤2.5 × ULN or ≤5 × |
| | ULN in the presence of liver metastases, and |
| | iii. aspartate aminotransferase (AST) ≤ 2.5 × ULN or ≤5 × ULN |
| | in the presence of liver metastases, and |
| | iv. international normalized ratio (INR) ≤ 2. |
| | c. Cardiac function as follows: |
| | i. left ventricular ejection fraction ≥ 50% |
| | ii. mean resting corrected QT interval (QTc) < 470 msec. |
| | 10. Acceptable hematologic function in the absence of growth factors |
| | and blood transfusions within 14 days: |
| | a. Absolute neutrophil count (ANC) ≥ 1,000 cells/mm³, and |
| | b. Hemoglobin ≥ 9 g/dL, and |
| | c. Platelet count ≥ 75,000 cells/mm³. |
| | Exclusion Criteria: |
| | 1. Treatment with any of the following within 14 days prior to the first |
| | administration of |
| | Compound (1-g): |
| | a. any systemic therapy for the treatment of advanced or |
| | metastatic breast cancer (the use of bisphosphonates is |
| | permitted), |
| | b. radiation therapy (prior palliative radiation is permitted >7 days), |
| | c. major surgery. |
| | 2. >3 lines of prior systemic chemotherapy in the advanced or metastatic setting |
| | (Note: there is no limit to the number of prior lines of endocrine therapy) |
| | 3. Known symptomatic brain metastases or those requiring ongoing |
| | treatment with |
| | corticosteroids (Note: Asymptomatic and treated, or asymptomatic untreated brain |
| | metastases are allowed as long as patients are clinically stable. Stable doses of |
| | anticonvulsants |
| | are allowed.) |
| | 4. Inability to swallow whole pills or any impairment of gastrointestinal function |
| | that may |
| | interfere with absorption, distribution, or metabolism of compound (1-g). |
| | 5. Any of the following cardiac criteria currently or within the last 6 months: |
| | a. any clinically important abnormalities (as assessed by the Investigator) in |
| | rhythm, conduction, or morphology of resting electrocardiograms |
| | (ECGs), e.g., complete left bundle branch block, third-degree heart block, |
| | b. congestive heart failure: New York Heart Association Class II-IV, |
| | c. any factor that increases the risk of QTc prolongation or risk of |
| | arrhythmic events such as congenital long QT syndrome or any new |
| | concomitant medication known to prolong the QT interval. |
| | 6. Any ongoing severe or uncontrolled systemic diseases (e.g. uncontrolled |
| | hypertension, uncontrolled diabetes mellitus, active bleeding diatheses, and any |
| | serious active infection requiring systemic therapy) |
| | 7. Active HIV infection defined by a history of AIDS-defining illness or HIV |
| | infection with a CD4+ T cell count < 350 cells/μL and an HIV viral load more than |
| | 400 copies/μL. |
| | 8. Patients with active viral (any etiology) hepatitis are excluded (patients with a |
| | history of viral hepatitis and a current viral load below the limit quantification in |
| | the absence of ongoing treatment may be eligible with approval from the Medical |
| | Monitor). |
| | 9. Any ongoing unresolved toxicities from prior treatment that are >Grade 1 as |
| | defined by National Cancer Institute (NCI) Common Terminology Criteria for |
| | Adverse Events (CTCAE) v5.0 or later at the time of study treatment, with the |
| | exception of alopecia, Grade 2 peripheral neuropathy and asymptomatic or |
| | adequately managed chronic Grade 2 toxicities. |
| | 10. Diagnosis of other active invasive cancers other than breast cancer within 2 |
| | years, exceptions include localized tumors cured with local treatment. |
| | 11. Prior history of allergic reaction to the composition or excipients of compound |
| | (1-g). |

c. Study Design

The phase 2 trial of compound (1-g) monotherapy is designed to evaluate patients with ER+/human epidermal growth factor receptor 2 negative (HER2−) advanced or metastatic breast cancer and a known ESR1 mutation who have received at least one line of endocrine therapy including a CDK4/6 inhibitor in the advanced or metastatic setting.

Participants are randomized 1:1 to Cohort A: 200 mg or Cohort B: 400 mg and stratified based on time on prior therapy with a CDK4/6 inhibitor (<12 months or ≥12 months) and the presence of measurable lesions (yes or no). A minimum of 30 participants in the trial are required to have at least 1 measurable lesion.

Pharmacokinetic (PK) and ECG assessments occur Day 1 and Day 15. At Cycle 1 on Day 1 and Day 15, PK and ECG assessments begin before taking compound (1-g) (pre-dose) and are collected intermittently for 24 hrs post-dose. The remaining cycles have 1 pre-dose PK sample on Day 1 of each cycle listed below.

Additional unscheduled PK samples may be drawn during the study for safety concerns. Should a participant require a dose change, additional PK samples to ensure safety and efficacy parameters may be required. At the first cycle following the dosing change, participants may be required to collect additional PK sampling at the same time points as Cycle 1 Day 1 and Cycle 1 Day 15. Subsequent pre-dose PK samples may be requested at the next 3 cycles following the dosing change.

Each participant who enters the screening phase for the study receives a unique participant identification number before any study-related activities/procedures are performed. This number is used to identify the participant throughout the clinical study and must remain constant throughout the clinical study; it must not be changed after initial assignment.

Eligibility information for each participant must be provided to the study Medical Monitor or designee and reviewed and approved prior to randomization. Participants are considered enrolled once they have been randomized and assigned to a cohort.

Approximately 60 eligible participants are randomized in a 1:1 ratio to either:
Cohort A: 200 mg once-daily orally (PO); or
Cohort B: 400 mg once-daily orally (PO).

Randomization is stratified by 2 factors: the presence of at least 1 measurable target lesion and time on prior CDK4/6 inhibitor therapy in the advanced/metastatic setting (<12 months and >12 months). A maximum of 30 participants without measurable disease is randomized between the two cohorts. After the maximum number is reached, participants without measurable disease are not to be permitted to be screened.

Study treatment commences within 7 calendar days of randomization according to treatment group assignment. Initiation of study treatment (Cycle 1 Day 1) delay >7 days after randomization must be approved by the study Medical Monitor.

d. Study Intervention

Compound (1-g) is an orally bioavailable ER degrader that contains ligands that bind human ERa and cereblon E3 ligase for the treatment of ER+/HER2− breast cancer. All participants receive compound (1-g) monotherapy at either 200 mg or 400 mg. Study treatment is administered in 28-day cycles using a “rolling calendar” meaning that cycle start dates occurs at 28-day intervals based on the first day of treatment with compound (1-g) (Cycle 1, Day 1). If study drug is held or a dose is missed, the study calendar continues to move forward without a pause and the next cycle start, with corresponding assessments, will still occur approximately 28 days from the previous one.

e. Study Assessments

Each participant's demographics, relevant medical history, and cancer history are collected. All prior anti-cancer treatment, including all surgical, radiotherapy, systemic therapy, and investigational therapies will be collected. Concomitant medications, including herbal supplements, over-the-counter medications, vitamins, and minerals, are reviewed at every visit and recorded. This includes all medications used within 30 days prior to Screening and all medications used from Screening until EOT.

Safety assessments are performed at every study visit as detailed in the SOA. At each study visit, a history and physical examination is performed, concomitant medications are reviewed, and evaluate any AEs are evaluated. In addition, vital signs, laboratory evaluations, and ECGs (in triplicate) are performed as scheduled to monitor organ function, hematologic function, and cardiovascular parameters.

A full physical examination includes vital signs, medical history height (cm), weight (kg) and a total body examination of all major body systems (general appearance, heart, lung, abdominal, head and neck, neurological, dermatological, and extremities). Post-Screening examinations may be targeted based on findings at Screening or participant complaints. Targeted physical exams include the collection of vital signs. Vital signs are performed prior to blood draws at a given time point, so as not to artificially alter these readings. Physical examinations and vital signs are collected per the SOA. Any new clinically significant findings from physical examinations are considered AEs.

Female participants undergo testing for estradiol and FSH at Screening only if required to meet the definition of postmenopausal per the inclusion criteria. Participants who meet one of the other inclusion criteria for menopausal status do not need this testing drawn at screening. Hematology labs are collected per the Schedule of Assessments and as needed to monitor for toxicities. Hematology assessments include white blood cell count, hemoglobin, mean corpuscular volume, platelets, absolute lymphocyte count, absolute neutrophil count, and reticulocyte count. Biochemistry assessments are collected per the SOA and as need to monitor for toxicities. Participants do not need to be fasted for blood draws. Biochemistry labs include sodium, potassium, chloride, bicarbonate (or equivalent), blood urea nitrogen, creatinine, calcium, phosphorous (or phosphate), magnesium, glucose, ALT, AST, alkaline phosphatase, total bilirubin, albumin, total protein, lactate dehydrogenase, and uric acid. Coagulation laboratory assessments include INR and partial thromboplastin time (PTT; or equivalent). Twelve-lead ECGs are performed prior to blood draws at a given time point, so as not to artificially alter these readings.

The schedule of assessments of a randomized Phase 2 study evaluating two dose levels of compound (1-g) in patients with ESR1 mutated ER+/HER2-advanced or metastatic breast cancer is presented in Table 17. Study visits are based on rolling calendar days from the start of treatment on Cycle 1 Day 1.

TABLE 17
The schedule of assessments of a randomized Phase 2 study evaluating two dose levels of compound
(1-g) in patients with ESR1 mutated ER+/HER2− advanced or metastatic breast cancer

| | Study Timepoint | | | | | | | | |
|---|---|---|---|---|---|---|---|---|---|
| | | | Cycle 1 | | | | | 30 d | |
| | | | (1 cycle = | | | | | Safety | Ext. |
| | Screening | | 28 days) | | Cycle 2 | Cycle 3+ | EOT^{q} | f/u^{r} | f/u^{s} |
| | Treatment Day | | | | | | | | |
| | Up to | Up to | | D 15 | D 1 | D 1 | ±7 | ±7 | |
| | Day −28 | Day −7 | D 1 | (±3) | (±3) | (±3) | Days | Days | |
| Informed Consent | X | | | | | | | | |
| Inclusion/Exclusion Criteria | X | | | | | | | | |
| Medical History and | X | | | | | | | | |
| Demographics | | | | | | | | | |
| Menopausal Status^{a} | X | | | | | | | | |
| (FSH and estradiol if | | | | | | | | | |
| indicated) | | | | | | | | | |
| HBV, HCV, HIV Testing^{b} | X | | | | | | | | |
| Echocardiogram^{c} | X | | | | | | | | |
| Physical Examination, | X | | X | X | X | X | X | X | |
| ECOG Performance Status | | | | | | | | | |
| Vital Signs^{d}, Height^{e}, | X | | X | X | X | X | X | X | |
| Weight | | | | | | | | | |
| Randomization | | X | | | | | | | |
| Serum Pregnancy Test^{f} | | X | X | | X | X | X | | |
| (if pre- or peri-menopausal) | | | | | | | | | |
| Safety Laboratories^{g} | | X | X | X | X | X | X | X | |
| (Hematology, Coagulation, | | | | | | | | | |
| Biochemistry) | | | | | | | | | |
| 12-lead ECGs^{h} (triplicate) | | X | X | X | X | X | | | |
| | | | | | | (required | | | |
| | | | | | | C3, C4, | | | |
| | | | | | | C6) | | | |
| Adverse Events | Collected from the date of ICF signature (research-related only), | | | | | | | | |
| (including SAEs) | during treatment and until 30 days after End of Treatment | | | | | | | | |
| | visit or until the start of a new anti-cancer agent | | | | | | | | |
| Pharmacokineticsⁱ | | | X | X | X | X | | | |
| | | | | | | (required | | | |
| | | | | | | C3, C4, | | | |
| | | | | | | C6) | | | |
| (Optional) Tumor Biopsy^{j} | X | | | | X | | | | |
| | | | | | (+27 | | | | |
| | | | | | days) | | | | |
| Blood Sample for ctDNA^{k} | | | X | | X | | X^{k} | | |
| | | | pre- | | | | | | |
| | | | dose | | | | | | |
| Blood Sample for | | | X | | X | X^{l} | X^{l} | | |
| Circulating Tumor Cells | | | pre- | | | (even | | | |
| (CTC)^{l} | | | dose | | | cycles | | | |
| | | | | | | only) | | | |
| Tumor Assessments^{m} | X | | | | | X | X | | X |
| | | | | | | (±5 days) | | | |
| | | | | | | (odd | | | |
| | | | | | | cycles | | | |
| | | | | | | only) | | | |
| Concomitant Medicationsⁿ | X | | | | | | | Xⁿ | |
| Review Dosing Diary | | | | X | X | X | X | | |
| Compound (1-g) Dosing^{o} | | | X | X | X | X | | | |
| Survival Status^{p} | | | | | | | | X | X |

CTC = circulating tumor cells;
ctDNA = circulating tumor deoxyribonucleic acid;
ECG = electrocardiogram;
ECOG = Eastern Cooperative Oncology Group;
EOT = end of treatment;
Ext f/u = extended follow up,
FSH = follicle-stimulating hormone;
ICF = informed consent form;
HBV = hepatitis B virus;
HCV = hepatitis C virus;
HIV = human immunodeficiency virus;
SAE = serious adverse event

f. Pharmacokinetics

PK assessments are collected at specific timepoints. Plasma PK parameters (including AUC₀₋₂₄, Cₘₐₓ, time to peak concentration, and half-life) are derived using data from serial testing on Cycle 1 Day 1 and Day 15. Steady-state PK characterization is informed by singular pre-dose blood draws on the first day of each cycle.

Frequency and timing of PK samples may be altered due to emerging data. If a PK timepoint is no longer needed, it may be eliminated from the PK schedule. Additional unscheduled PK samples may be drawn during the study for safety concerns. In addition, should a participant require a dose change, additional PK samples to ensure safety and efficacy parameters may be required. At the first cycle following the dosing change, participants may be required to collect additional PK sampling at the same time points as Cycle 1 Day 1 and Cycle 1 Day 15. Subsequent pre-dose PK samples may be requested at the next 3 cycles following the dosing change. Decisions as to whether or not these samples are to be collected will be determined in consultation with the Medical Monitor.

g. Tumor biomarkers

Paired tumor biopsies provide important pharmacodynamic parameters that may be predictive of compound (1-g) response. Paired biopsies are optional and are used for exploratory analysis. Biopsies occur pre-treatment and during Cycle 2.

Lesions used for the biopsies must be accessible. Biopsies should be taken from the same lesion each time, if feasible. Analysis of the samples will be done retrospectively to evaluate ER degradation by immunohistochemistry.

Blood samples for ctDNA are collected according to the Schedule of Assessments. The ctDNA sample at Cycle 1 Day 1 retrospectively confirms ESR1 mutation status. Other alterations in genes relevant to breast cancer may also be explored through ctDNA. On Cycle 1 Day1, this sample should be collected prior to the first dose of compound (1-g). Exploratory efficacy assessments include ctDNA.

Circulating tumor cell samples are collected at specific timepoints. On Cycle 1 Day 1, a sample should be collected prior to the first dose of compound (1-g).

Tumor assessments are completed within the appropriate window and timepoints. Imaging assessments and responses are performed in accordance with RECIST version 1.1. All participants have baseline tumor assessments within 28 days prior to the planned first dose of compound (1-g) and assessment of measurable lesions must be completed prior to randomization. The imaging modalities used for tumor assessments to follow a given target or non-target lesion throughout the study should be consistent with the modality used at Screening. Each tumor response assessment includes evaluation of target lesions (complete response [CR], partial response [PR], SD, progressive disease [PD], or not evaluable [NE]), non-target lesions (CR, Non-CR/Non-PD, PD, or NE), and overall timepoint response (CR, PR, SD, Non-CR/Non-PD, PD, or NE).

h. Dosing and Administration

Participants receive study treatment at a dose level determined by randomization until disease progression, unacceptable toxicity, withdrawal of consent, or death (whichever occurs first).

Compound (1-g) is administered to participants in the morning with food. The tablets should be swallowed whole with liquid. The time of day for administration of compound (1-g) should be consistent. The participant is provided with a medication diary to record daily dosing. Participants must avoid taking other medications at the same time as compound (1-g). Other medications should be taken in the evening, or at least 4 hours after taking compound (1-g).

If the participant misses a dose of study drug, the participant should take the dose as soon as possible, but not less than 12 hours before the next dose is due. If the next dose is due in less than 12 hours, the participant should skip the missed dose and take the next dose as scheduled.

If vomiting occurs after taking the study treatment, the participant should be instructed not to retake the dose. Participants should take the next scheduled dose of compound (1-g). If vomiting persists, the participant should contact the Investigator.

On PK collection visits, participants should be instructed to bring the study medication with them to the clinic and will take the dose after the pre-dose PK has been taken.

i. Formulation

Compound (1-g) is supplied in a tablet formulation of 25 mg and 100 mg. Compound (1-g) is packaged in high density polyethylene bottles. The immediate packaging contains a statement to conform with FDA Investigational New Drug requirements as follows: “Caution: New Drug-Limited by federal (or United States) law to investigational use.”

Storage conditions for compound (1-g) require the tablets to be stored at 15° C. to 25° C. (59° F. to 77° F.) and protected from light.

j. Statistical Considerations

A sample size of 30 participants per cohort provides a clinical meaningful assessment of compound (1-g) at each dose. As an approximation and assuming a binomial distribution, the PFS rate at 3 or 6 months may be estimated with a maximum half-width of 18.7%. For example, if 15 out of 30 participants are alive and progression free at 3 months, the PFS rate is 50% with a 95% CI of (31.3%, 68.7%).

The statistical analyses of the study are descriptive. The primary focus is on the cohort-specific analysis. A comparison between cohorts is provided. Point estimates are accompanied by 95% confidence intervals (CIs). Hypothesis testing, if performed, is considered exploratory and assessed at the conventional 2-sided 5% significance level without multiplicity adjustment.

The primary efficacy endpoint of PFS is defined as the time from the date of randomization until the date of progression (per Investigator's assessment based on RECIST v1.1) or death due to any cause, whichever occurs earlier. Participants who are alive without progression will be censored. The PFS analysis censoring rules will be detailed in the SAP.

The analysis of PFS will be based on the mITT population. Kaplan-Meier methodology will be used to estimate PFS probabilities at 6 months and the associated 95% CI by treatment cohort. Estimated PFS probabilities and 95% CIs at additional landmark timepoints (e.g., 3, 9 and 12 months) will also be provided. The median PFS and its 95% CI will be calculated according to the Brookmeyer-Crowley methodology for each treatment cohort.

As an exploratory analysis, a stratified log-rank test is performed to compare the PFS endpoint between the two treatment cohorts. A stratified Cox proportional hazards model is used to estimate the hazard ratio and its 95% CI. Objective response rate (ORR) is another primary efficacy endpoint. ORR is defined as the proportion of participants achieving confirmed CR or PR per Investigator's assessment based on RECIST v1.1. The ORR analysis is based on the Response Evaluable Population. The ORR estimate for each treatment Cohort is provided along with the Clopper-Pearson 95% CI.

Secondary efficacy endpoints based on the tumor assessments are defined as follows: Clinical benefit rate (CBR) is defined as the proportion of participants achieving confirmed CR, or PR, or SD≥24 weeks, per Investigator assessment based on RECIST v1.1; Duration of Response (DOR) is defined as the time between first documentation of a response (CR or PR) and first evidence of PD according to RECIST v1.1 or death due to any cause. The CBR is analyzed based on the Response Evaluable Population. The CBR is estimated for each treatment Cohort accompanied by 95% Clopper-Pearson CI. The between Cohort difference in CBR will also be provided along with its 95% CI. Duration of response (DOR) is calculated for responders. The Kaplan-Meier methodology is used to analyze the DOR endpoint. The median DOR (if estimable) and percentage of participants still in response at selected timepoints is estimated along with the 95% CIs.

### EQUIVALENTS AND INCORPORATION BY REFERENCE

While aspects of the present disclosure have been particularly shown and described with reference to certain embodiments and various alternate embodiments, it will be understood by persons skilled in the relevant art that various changes in form and details can be made therein without departing from the spirit and scope of the present disclosure.

All publications, patents, and patent applications mentioned in this specification are herein incorporated by reference to the same extent as if each individual publication, patent, or patent application was specifically and individually indicated as being incorporated by reference herein, including, for example, international patent application nos. PCT/US2020/027895 (published as WO 2021/118629) and PCT/US2020/066798 (published as WO 2021/133886) and U.S. provisional patent application Nos. 63/650,793 and 63/691,567.

1. A method of treating breast cancer in a patient in need thereof, comprising administering to the patient a therapeutically effective amount of a compound, wherein the compound is represented by Formula 1 or is a pharmaceutically acceptable salt thereof: 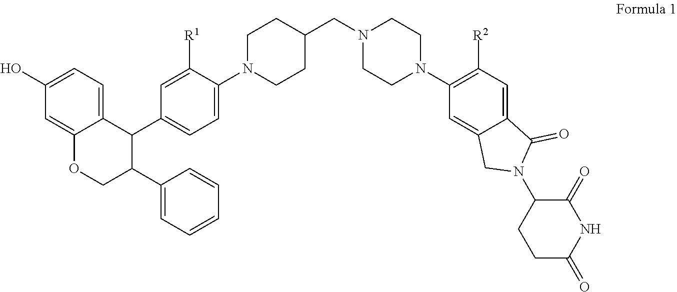
wherein:
R¹ is selected from H, Cl, Br, and F, and
R² is H or F, and
wherein the therapeutically effective amount of the compound of Formula 1 is 20 mg to 1200 mg.

2-10. (canceled)

11. The method of claim 1, wherein the compound of Formula 1 is selected from:

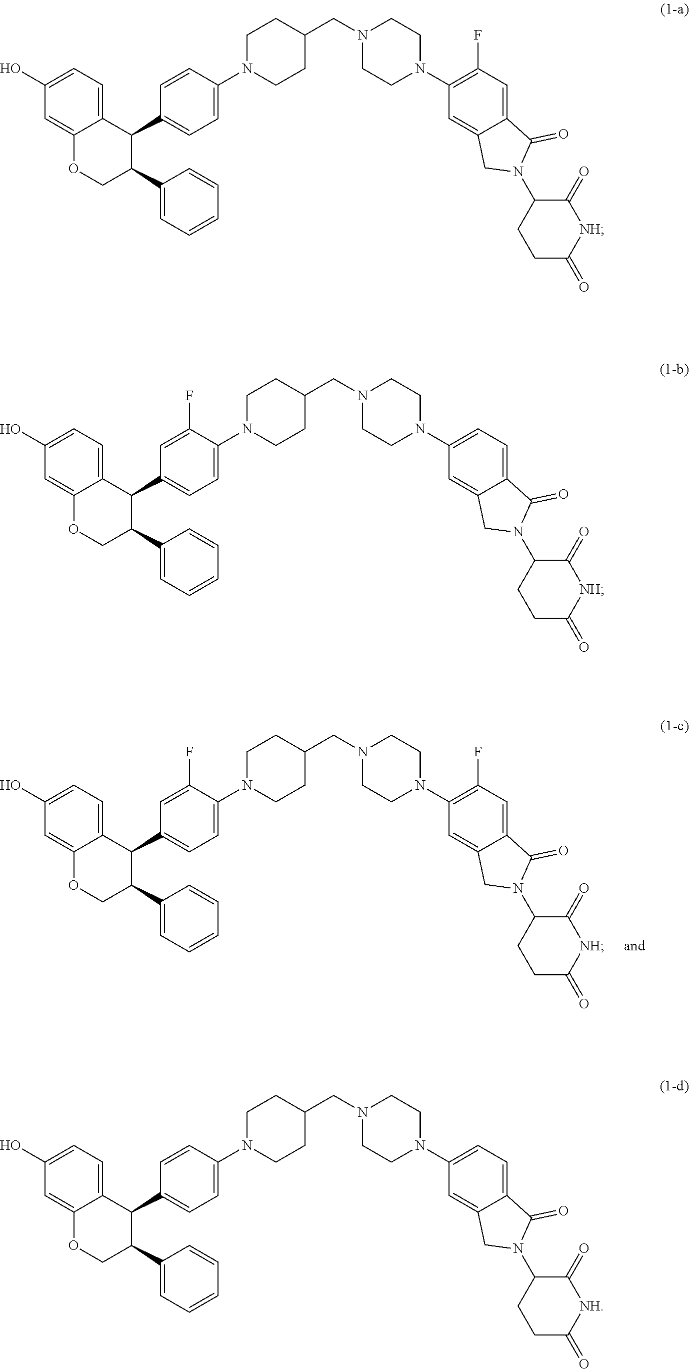 [SMILES: O=C1CCC(N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CCC(N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; "and" ; O=C1CCC(N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

12-15. (canceled)

16. The method of claim 1, wherein the compound of Formula 1 is 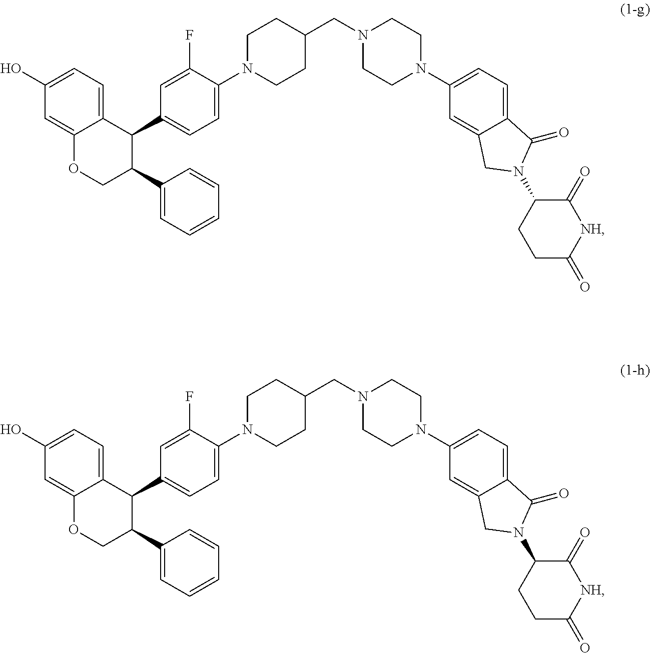 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]
or a combination thereof.

17. The method of claim 16, wherein the compound of Formula 1 is a mixture of compound (1-g) and compound (1-h).

18. The method of claim 17, wherein the relative amounts of compound (1-g) and (1-h) in the mixture are greater than 80:20.

19. The method of claim 17, wherein the mixture comprises less than 10% by weight of compound (1-h).

20-28. (canceled)

29. The method of claim 1, wherein the compound of Formula 1 is

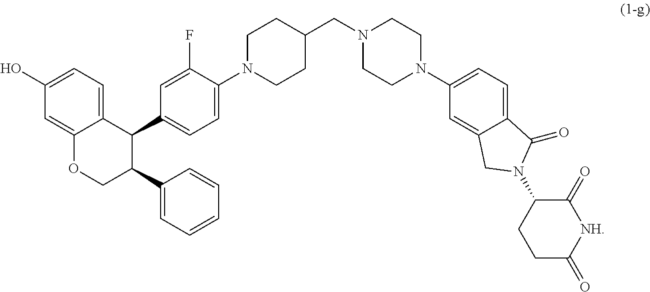 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

30-57. (canceled)

58. The method of claim 1, wherein the breast cancer comprises an Estrogen Receptor 1 (ESR1) tumor mutation or ESR1-wild-type, the method comprising:
orally administering to the patient once daily a therapeutically effective amount of a compound, wherein the compound is selected from the following: 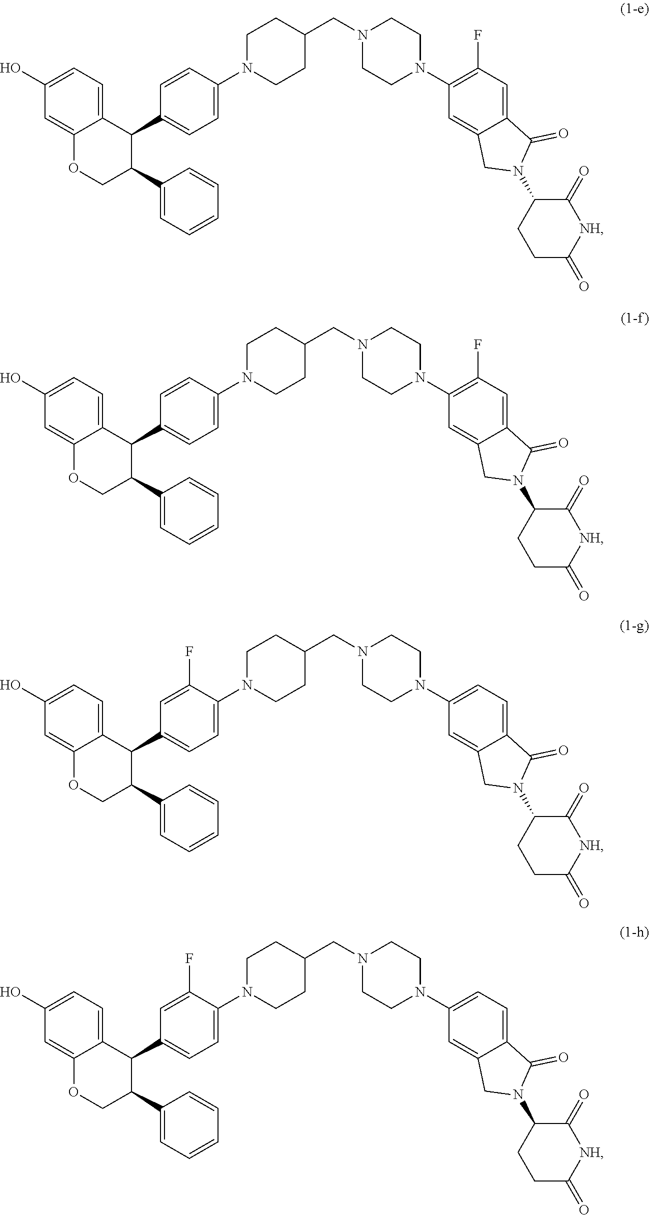 [SMILES: O=C1CC[C@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1] 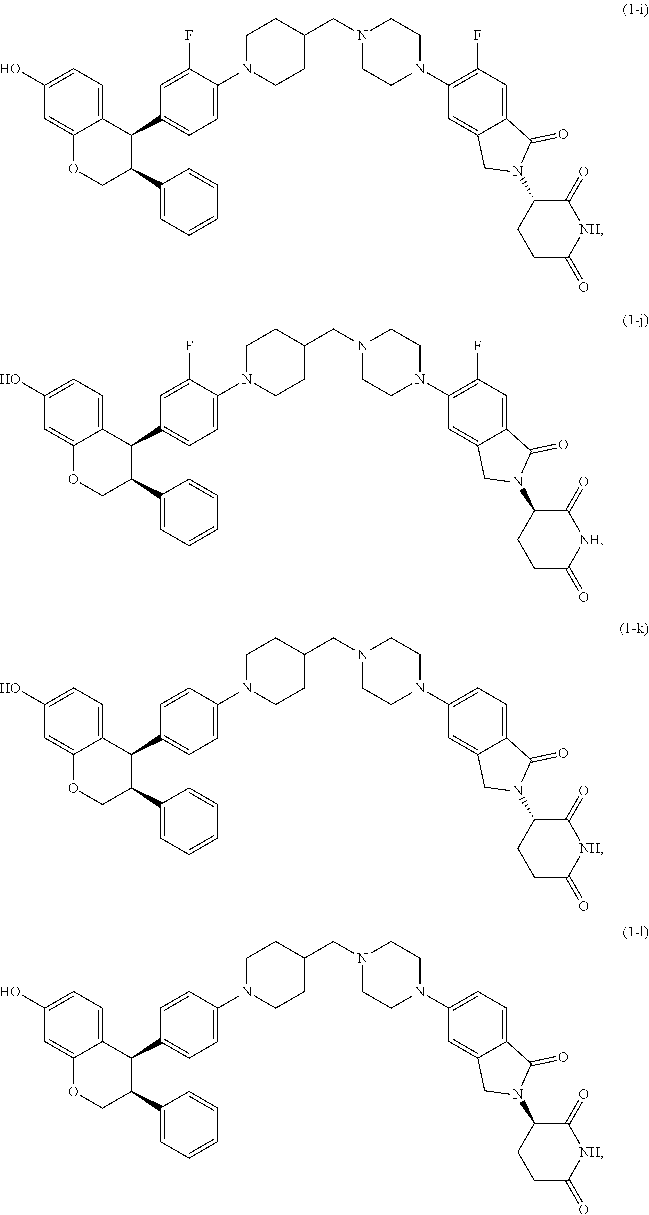 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6F)CC5)CC4)c(F)cc3C2=O)C(=O)N1 ; O=C1CC[C@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1 ; O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]
and
combinations thereof, or is a pharmaceutically acceptable salt thereof.

59-61. (canceled)

62. The method of claim 58, wherein the compound of Formula 1 is a mixture of compound (1-g) and compound (1-h).

63. The method of claim 62, wherein the relative amounts of compound (1-g) and (1-h) in the mixture are greater than 80:20.

64. The method of claim 62, wherein the mixture comprises less than 10% by weight of compound (1-h).

65-71. (canceled)

72. The method of claim 58, wherein the compound is

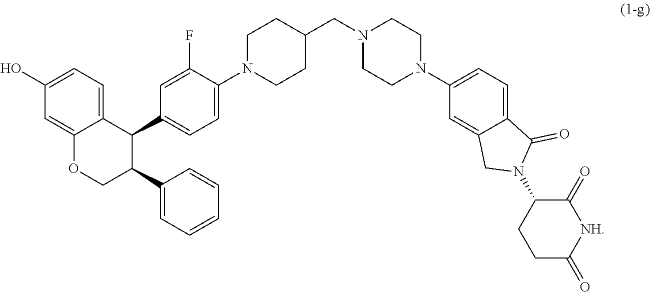 [SMILES: O=C1CC[C@H](N2Cc3cc(N4CCN(CC5CCN(c6ccc([C@@H]7c8ccc(O)cc8OC[C@@H]7c7ccccc7)cc6F)CC5)CC4)ccc3C2=O)C(=O)N1]

73-74. (canceled)

75. The method of claim 58, wherein the at least one ESR1 tumor mutation is V422del or selected from Y537Z, D538Z, E380Z, L379Z, S463Z, and L536Z, wherein “Z” is an amino acid residue other than the wild-type residue at that position.

76. The method of claim 75, wherein the at least one ESR1 tumor mutation is selected from Y537S, Y537N, Y537C, D538G, E380Q, L379I, S463P, L536P, L536H, and L536R.

77. The method of claim 58, wherein the breast cancer is estrogen receptor-positive/human epidermal growth factor receptor 2-negative (ER+/HER2−).

78-79. (canceled)

80. The method of claim 58, wherein the therapeutically effective amount of the compound is 100 mg to 600 mg.

81-115. (canceled)

116. A compound, wherein the compound is of the following structure:

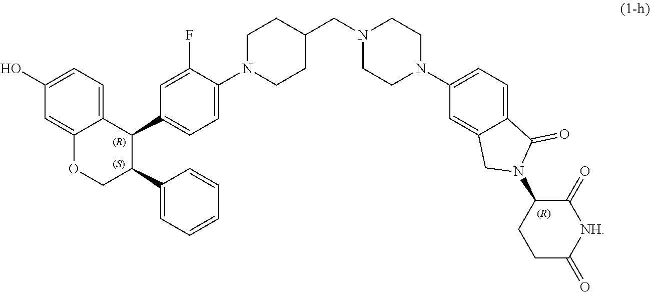

117-121. (canceled)

122. A mixture of the following compounds

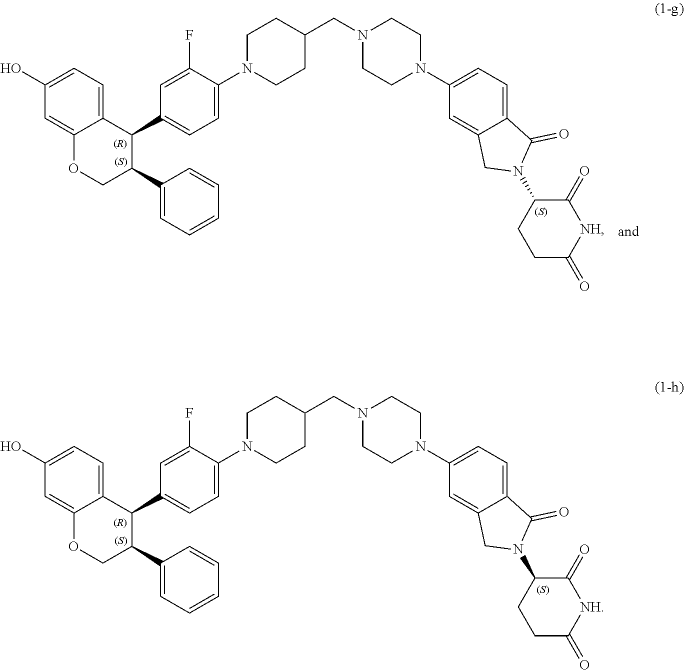

123. The mixture of claim 122, wherein the relative amounts of compound (1-g) and (1-h) in the mixture are greater than 80:20.

124. The mixture of claim 122, wherein the mixture comprises less than 10% by weight of compound (1-h).

125-130. (canceled)

131. A pharmaceutical composition comprising the mixture of claim 122 and a pharmaceutically acceptable carrier.
